SEQUENCE LISTING (43 sequences)

SEQ ID NO: 1 - protein, 830 aa, synthetic construct
  REGION 1..830: note = synthetic sequence
MALPVTALLLPLALLLHAARPDIVMTQSPDSLAVSLGERVTMNCKSSQSLLYSTNQKNYLAWYQQKPGQSPKLLIYWASTRESGVPDRFSGSGSGTDFTLTISSVQAEDVAVYYCQQYYSYRTFGGGTKLEIKGGGGSGGGGSGGGGSQVQLQQSGPEVVKPGASVKMSCKASGYTFTSYVIHWVRQKPGQGLDWIGYINPYNDGTDYDEKFKGKATLTSDTSTSTAYMELSSLRSEDTAVYYCAREKDNYATGAWFAYWGQGTLVTVSSTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGATNFSLLKQAGDVEENPGPMYRMQLLSCIALSLALVTNSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSLQAPRRARGCRTLGLPALLLLLLLRPPATRGITCPPPMSVEHADIWVKSYSLYSRERYICNSGFKRKAGTSSLTECVLNKATNVAHWTTPSLKCIR

SEQ ID NO: 2 - DNA, 2509 bp, synthetic construct
  misc_feature 1..2509: note = synthetic sequence
  source 1..2509: mol_type = other DNA
gcgatcgcatggccttaccagtgaccgccttgctcctgccgctggccttgctgctccacgccgccaggccggacatcgtgatgacccaaagccccgacagcctggccgtgagcctgggcgagagggtgaccatgaactgcaaaagcagccagtccctgctgtactccaccaaccagaagaactacctggcttggtatcaacagaagcccggacagagccccaagctgctgatctattgggccagcactagggaaagcggcgtgcccgataggttcagcggcagcgggagcggcacagacttcactctgaccattagcagcgtgcaggctgaggatgtggccgtctactactgccagcagtactacagctacaggacctttgggggcggaactaagctggagatcaagggaggggggggatccgggggaggaggctccggcggaggcggaagccaagtgcaactgcagcagagcggcccagaggtggtcaaacctggggcaagcgtgaagatgagctgcaaggctagcggctataccttcaccagctatgtgatccactgggtgaggcagaaaccaggacagggcctggactggatcggctacatcaacccctacaatgacggcaccgattatgacgaaaaattcaaggggaaggccaccctgaccagcgacaccagcacaagcaccgcctacatggagctgtccagcctgaggtccgaggacaccgccgtgtattactgtgccagggagaaggacaattacgccaccggcgcttggttcgcctactggggccagggcacactggtgacagtgagcagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaggagtaagaggagcaggctcctgcacagtgactacatgaacatgactccccgccgccccgggcccacccgcaagcattaccagccctatgccccaccacgcgacttcgcagcctatcgctccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggaagcggagccaccaacttcagcctgctgaagcaggccggcgacgtggaggagaaccccggccccatgtacagaatgcagctgctgagctgcatcgccctgagcctggccctggtgaccaacagcggcatccacgtgttcatcctgggctgcttcagcgccggcctgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgcttcctgctggagctgcaggtgatcagcctggagagcggcgacgccagcatccacgacaccgtggagaacctgatcatcctggccaacaacagcctgagcagcaacggcaacgtgaccgagagcggctgcaaggagtgcgaggagctggaggagaagaacatcaaggagttcctgcagagcttcgtgcacatcgtgcagatgttcatcaacaccagctccggcggcggctccggcggcggcggctccggcggcggcggctccggcggcggcggctccggcggcggctccctgcaggcccccagaagagccagaggctgcagaaccctgggcctgcccgccctgctgctgctgctgctgctgagaccccccgccaccagaggcatcacctgccccccccccatgagcgtggagcacgccgacatctgggtgaagagctacagcctgtacagcagagagagatacatctgcaacagcggcttcaagagaaaggccggcaccagcagcctgaccgagtgcgtgctgaacaaggccaccaacgtggcccactggaccacccccagcctgaagtgcatcagataagtttaaac

SEQ ID NO: 3 - protein, 995 aa, synthetic construct
  REGION 1..995: note = synthetic sequence
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGATNFSLLKQAGDVEENPGPMALPVTALLLPLALLLHAARPQIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKGGGGSGGGGSGGGGSQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSATTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 4 - DNA, 3004 bp, synthetic construct
  misc_feature 1..3004: note = synthetic sequence
  source 1..3004: mol_type = other DNA
gcgatcgcatggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggaagcggagccaccaacttcagcctgctgaagcaggccggcgacgtggaggagaaccccggccccatggccttaccagtgaccgccttgctcctgccgctggccttgctgctccacgccgccaggccgcagatcgtgctgagccagagccctgccatcctgtccgcaagcccaggcgagaaggtgaccatgacctgtagggccagcagctccgtgagctacatccactggtttcagcagaagcctggaagcagccctaagccctggatctacgccacaagcaatctggctagcggcgtgcccgtgaggttcagcggcagcgggagcgggaccagctacagcctgactatcagcagggtggaggccgaggacgccgccacatactactgccaacagtggacctccaacccacccacctttggaggagggacaaaactggagatcaaagggggcggagggtccggaggcggcggaagcgggggagggggaagccaggtccaactgcaacagcccggagcagaactggtcaaaccaggcgccagcgtgaagatgagctgcaaggccagcgggtacaccttcacttcctataacatgcactgggtgaagcagaccccaggaaggggcctggagtggatcggggcaatctatcccggcaacggcgacacaagctacaaccagaagttcaaggggaaagccactctgaccgccgacaagtccagctccaccgcctacatgcagctgagctccctgaccagcgaggacagcgccgtgtactattgcgccagaagcacttattacggaggggactggtacttcaacgtgtggggggcagggaccaccgtgaccgtgtccgccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaagtttaaac

SEQ ID NO: 5 - protein, 1001 aa, synthetic construct
  REGION 1..1001: note = synthetic sequence
DRMALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMALPVTALLLPLALLLHAARPDIQMTQSPSSLSASVGDRVTITCRSSQSIVHSVGNTFLEWYQQKPGKAPKLLIYKVSNRFSGVPSRFSGSGSGTDFTLTISSLQPEDFATYYCFQGSQFPYTFGQGTKVEIKGGGGSGGGGSGGGGSEVQLVESGGGLVQPGGSLRLSCAASGYEFSRSWMNWVRQAPGKGLEWVGRIYPGDGDTNYSGKFKGRFTISADTSKNTAYLQMNSLRAEDTAVYYCARDGSSWDWYFDVWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 6 - DNA, 3016 bp, synthetic construct
  misc_feature 1..3016: note = synthetic sequence
  source 1..3016: mol_type = other DNA
gcgatcgcatggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggaagcggagccaccaacttcagcctgctgaagcaggccggcgacgtggaggagaaccccggccccatggccttaccagtgaccgccttgctcctgccgctggccttgctgctccacgccgccaggccggatatccagatgacccagagccccagctccctgtccgcatccgtgggcgacagagtgacaattacctgtagaagcagccaaagcatcgtgcatagcgtcggcaacacttttctggagtggtatcaacagaagcccgggaaggcccccaaactgctgatctacaaggtgagcaacagattcagcggggtcccaagcagattctccggcagcggctccgggactgacttcaccctgaccattagcagcctgcagccagaggacttcgccacatactactgcttccaagggagccagttcccctacaccttcggccaaggcactaaggtggagatcaaaggggggggaggaagcggcggaggagggagcggaggcgggggatccgaagtgcaactggtcgaatccggaggggggctggtccagcctggagggtccctgagactgagctgcgccgcaagcggctacgagttctccaggtcctggatgaactgggtgaggcaggccccaggaaaagggctggaatgggtgggcaggatctaccctggcgacggcgataccaactactccggaaagttcaagggcaggttcactatcagcgccgacactagcaagaataccgcctacctgcagatgaatagcctgagggccgaggacaccgccgtgtattactgcgctagagacggcagcagctgggattggtacttcgacgtgtggggccagggcactctggtgactgtgagcagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaagtttaaac

SEQ ID NO: 7 - protein, 990 aa, synthetic construct
  REGION 1..990: note = synthetic sequence
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGATNFSLLKQAGDVEENPGPMALPVTALLLPLALLLHAARPDVQITQSPSYLAASPGETITINCRASKSISKDLAWYQEKPGKTNKLLIYSGSTLQSGIPSRFSGSGSGTDFTLTISSLEPEDFAMYYCQQHNKYPYTFGGGTKLEIKGGGGSGGGGSGGGGSQVQLQQPGAELVRPGASVKLSCKASGYTFTSYWMNWVKQRPDQGLEWIGRIDPYDSETHYNQKFKDKAILTVDKSSSTAYMQLSSLTSEDSAVYYCARGNWDDYWGQGTTLTVSSTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 8 - DNA, 2989 bp, synthetic construct
  misc_feature 1..2989: note = synthetic sequence
  source 1..2989: mol_type = other DNA
gcgatcgcatggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggaagcggagccaccaacttcagcctgctgaagcaggccggcgacgtggaggagaaccccggccccatggccttaccagtgaccgccttgctcctgccgctggccttgctgctccacgccgccaggccggacgtgcagatcacccagagccccagctacctggccgccagccccggcgagaccatcaccatcaactgcagagccagcaagagcatcagcaaggacctggcctggtaccaggagaagcccggcaagaccaacaagctgctgatctacagcggcagcaccctgcagagcggcatccccagcagattcagcggcagcggcagcggcaccgacttcaccctgaccatcagcagcctggagcccgaggacttcgccatgtactactgccagcagcacaacaagtacccctacaccttcggcggcggcaccaagctggagatcaagggaggggggggatccgggggaggaggctccggcggaggcggaagccaggtgcagctgcagcagcccggcgccgagctggtgagacccggcgccagcgtgaagctgagctgcaaggccagcggctacaccttcaccagctactggatgaactgggtgaagcagagacccgaccagggcctggagtggatcggcagaatcgacccctacgacagcgagacccactacaaccagaagttcaaggacaaggccatcctgaccgtggacaagagcagcagcaccgcctacatgcagctgagcagcctgaccagcgaggacagcgccgtgtactactgcgccagaggcaactgggacgactactggggccagggcaccaccctgaccgtgagcagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaagtttaaac

SEQ ID NO: 9 - protein, 1066 aa, synthetic construct
  REGION 1..1066: note = synthetic sequence
MALPVTALLLPLALLLHAARPQIVLTQSPAIMSASPGEKVTITCSASSSISYMHWFQQKPGTSPKLWIYTTSNLASGVPARFSGSGSGTSYSLTISRMEAEDAATYYCHQRSTYPLTFGSGTKLELKGGGGSGGGGSGGGGSQVQLQQSGAELAKPGASVKMSCKASGYTFTSYRMHWVKQRPGQGLEWIGYINPSTGYTEYNQKFKDKATLTADKSSSTAYMQLSSLTFEDSAVYYCARGGGVFDYWGQGTTLTVSSTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMALPVTALLLPLALLLHAARPDVQITQSPSYLAASPGETITINCRASKSISKDLAWYQEKPGKTNKLLIYSGSTLQSGIPSRFSGSGSGTDFTLTISSLEPEDFAMYYCQQHNKYPYTFGGGTKLEIKGGGGSGGGGSGGGGSQVQLQQPGAELVRPGASVKLSCKASGYTFTSYWMNWVKQRPDQGLEWIGRIDPYDSETHYNQKFKDKAILTVDKSSSTAYMQLSSLTSEDSAVYYCARGNWDDYWGQGTTLTVSSTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 10 - DNA, 3217 bp, synthetic construct
  misc_feature 1..3217: note = synthetic sequence
  source 1..3217: mol_type = other DNA
gcgatcgcatggccttaccagtgaccgccttgctcctgccgctggccttgctgctccacgccgccaggccgcagatcgtgctgacccagagccccgccatcatgagcgccagccccggcgagaaggtgaccatcacctgcagcgccagcagcagcatcagctacatgcactggttccagcagaagcccggcaccagccccaagctgtggatctacaccaccagcaacctggccagcggcgtgcccgccagattcagcggcagcggcagcggcaccagctacagcctgaccatcagcagaatggaggccgaggacgccgccacctactactgccaccagagaagcacctaccccctgaccttcggcagcggcaccaagctggagctgaagggaggggggggatccgggggaggaggctccggcggaggcggaagccaggtgcagctgcagcagagcggcgccgagctggccaagcccggcgccagcgtgaagatgagctgcaaggccagcggctacaccttcaccagctacagaatgcactgggtgaagcagagacccggccagggcctggagtggatcggctacatcaaccccagcaccggctacaccgagtacaaccagaagttcaaggacaaggccaccctgaccgccgacaagagcagcagcaccgcctacatgcagctgagcagcctgaccttcgaggacagcgccgtgtactactgcgccagaggcggcggcgtgttcgactactggggccagggcaccaccctgaccgtgagcagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaggagtaagaggagcaggctcctgcacagtgactacatgaacatgactccccgccgccccgggcccacccgcaagcattaccagccctatgccccaccacgcgacttcgcagcctatcgctccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggaagcggagctactaacttcagcctgctgaagcaggctggagacgtggaggagaaccctggacctatggccttaccagtgaccgccttgctcctgccgctggccttgctgctccacgccgccaggccggacgtgcagatcacccagagccccagctacctggccgccagccccggcgagaccatcaccatcaactgcagagccagcaagagcatcagcaaggacctggcctggtaccaggagaagcccggcaagaccaacaagctgctgatctacagcggcagcaccctgcagagcggcatccccagcagattcagcggcagcggcagcggcaccgacttcaccctgaccatcagcagcctggagcccgaggacttcgccatgtactactgccagcagcacaacaagtacccctacaccttcggcggcggcaccaagctggagatcaagggaggggggggatccgggggaggaggctccggcggaggcggaagccaggtgcagctgcagcagcccggcgccgagctggtgagacccggcgccagcgtgaagctgagctgcaaggccagcggctacaccttcaccagctactggatgaactgggtgaagcagagacccgaccagggcctggagtggatcggcagaatcgacccctacgacagcgagacccactacaaccagaagttcaaggacaaggccatcctgaccgtggacaagagcagcagcaccgcctacatgcagctgagcagcctgaccagcgaggacagcgccgtgtactactgcgccagaggcaactgggacgactactggggccagggcaccaccctgaccgtgagcagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaggagtaagaggagcaggctcctgcacagtgactacatgaacatgactccccgccgccccgggcccacccgcaagcattaccagccctatgccccaccacgcgacttcgcagcctatcgctccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaagtttaaac

SEQ ID NO: 11 - protein, 985 aa, synthetic construct
  REGION 1..985: note = synthetic sequence
MALPVTALLLPLALLLHAARPQIVLTQSPAIMSASPGEKVTITCSASSSISYMHWFQQKPGTSPKLWIYTTSNLASGVPARFSGSGSGTSYSLTISRMEAEDAATYYCHQRSTYPLTFGSGTKLELKGGGGSGGGGSGGGGSQVQLQQSGAELAKPGASVKMSCKASGYTFTSYRMHWVKQRPGQGLEWIGYINPSTGYTEYNQKFKDKATLTADKSSSTAYMQLSSLTFEDSAVYYCARGGGVFDYWGQGTTLTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGATNFSLLKQAGDVEENPGPMALPVTALLLPLALLLHAARPDVQITQSPSYLAASPGETITINCRASKSISKDLAWYQEKPGKTNKLLIYSGSTLQSGIPSRFSGSGSGTDFTLTISSLEPEDFAMYYCQQHNKYPYTFGGGTKLEIKGGGGSGGGGSGGGGSQVQLQQPGAELVRPGASVKLSCKASGYTFTSYWMNWVKQRPDQGLEWIGRIDPYDSETHYNQKFKDKAILTVDKSSSTAYMQLSSLTSEDSAVYYCARGNWDDYWGQGTTLTVSSTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 12 - DNA, 2974 bp, synthetic construct
  misc_feature 1..2974: note = synthetic sequence
  source 1..2974: mol_type = other DNA
gcgatcgcatggccttaccagtgaccgccttgctcctgccgctggccttgctgctccacgccgccaggccgcagatcgtgctgacccagagccccgccatcatgagcgccagccccggcgagaaggtgaccatcacctgcagcgccagcagcagcatcagctacatgcactggttccagcagaagcccggcaccagccccaagctgtggatctacaccaccagcaacctggccagcggcgtgcccgccagattcagcggcagcggcagcggcaccagctacagcctgaccatcagcagaatggaggccgaggacgccgccacctactactgccaccagagaagcacctaccccctgaccttcggcagcggcaccaagctggagctgaagggaggggggggatccgggggaggaggctccggcggaggcggaagccaggtgcagctgcagcagagcggcgccgagctggccaagcccggcgccagcgtgaagatgagctgcaaggccagcggctacaccttcaccagctacagaatgcactgggtgaagcagagacccggccagggcctggagtggatcggctacatcaaccccagcaccggctacaccgagtacaaccagaagttcaaggacaaggccaccctgaccgccgacaagagcagcagcaccgcctacatgcagctgagcagcctgaccttcgaggacagcgccgtgtactactgcgccagaggcggcggcgtgttcgactactggggccagggcaccaccctgaccgtgagcagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggaagcggagctactaacttcagcctgctgaagcaggctggagacgtggaggagaaccctggacctatggccttaccagtgaccgccttgctcctgccgctggccttgctgctccacgccgccaggccggacgtgcagatcacccagagccccagctacctggccgccagccccggcgagaccatcaccatcaactgcagagccagcaagagcatcagcaaggacctggcctggtaccaggagaagcccggcaagaccaacaagctgctgatctacagcggcagcaccctgcagagcggcatccccagcagattcagcggcagcggcagcggcaccgacttcaccctgaccatcagcagcctggagcccgaggacttcgccatgtactactgccagcagcacaacaagtacccctacaccttcggcggcggcaccaagctggagatcaagggaggggggggatccgggggaggaggctccggcggaggcggaagccaggtgcagctgcagcagcccggcgccgagctggtgagacccggcgccagcgtgaagctgagctgcaaggccagcggctacaccttcaccagctactggatgaactgggtgaagcagagacccgaccagggcctggagtggatcggcagaatcgacccctacgacagcgagacccactacaaccagaagttcaaggacaaggccatcctgaccgtggacaagagcagcagcaccgcctacatgcagctgagcagcctgaccagcgaggacagcgccgtgtactactgcgccagaggcaactgggacgactactggggccagggcaccaccctgaccgtgagcagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaagtttaaac

SEQ ID NO: 13 - protein, 546 aa, synthetic construct
  REGION 1..546: note = synthetic sequence
MALPVTALLLPLALLLHAARPSDIVLTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIKKISGGGGSGGGGSGGGGSGGGGSGGGGSQVQLVESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 14 - DNA, 1657 bp, synthetic construct
  misc_feature 1..1657: note = synthetic sequence
  source 1..1657: mol_type = other DNA
gcgatcgcatggccttaccagtgaccgccttgctcctgccgctggccttgctgctccacgccgccaggccgagcgacatcgtgctgacccagagccccgccagcctggccgtgagcctgggccagagagccaccatcagctgcagagccagcaagagcgtgagcaccagcggctacagctacctgcactggtaccagcagaagcccggccagccccccaagctgctgatctacctggccagcaacctggagagcggcgtgcccgccagattcagcggcagcggcagcggcaccgacttcaccctgaacatccaccccgtggaggaggaggacgccgccacctactactgccagcacagcagagagctgcccttcaccttcggcagcggcaccaagctggagatcaagaagatcagcggcggcggcggcagcggcggcggcggcagcggcggcggcggcagcggcggcggcggcagcggcggcggcggcagccaggtgcagctggtggagagcggcggcggcctggtgcagcccggcggcagcctgaagctgagctgcgccgccagcggcttcgacttcagcagatactggatgagctgggtgagacaggcccccggcaagggcctggagtggatcggcgagatcaaccccaccagcagcaccatcaacttcacccccagcctgaaggacaaggtgttcatcagcagagacaacgccaagaacaccctgtacctgcagatgagcaaggtgagaagcgaggacaccgccctgtactactgcgccagaggcaactactacagatacggcgacgccatggactactggggccagggcaccagcgtgaccgtgagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaggagtaagaggagcaggctcctgcacagtgactacatgaacatgactccccgccgccccgggcccacccgcaagcattaccagccctatgccccaccacgcgacttcgcagcctatcgctccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaagtttaaac

SEQ ID NO: 15 - protein, 490 aa, synthetic construct
  REGION 1..490: note = synthetic sequence
MALPVTALLLPLALLLHAARPDIVLTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIKGGGGSGGGGSGGGGSQVQLVESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 16 - DNA, 1489 bp, synthetic construct
  misc_feature 1..1489: note = synthetic sequence
  source 1..1489: mol_type = other DNA
gcgatcgcatggccttaccagtgaccgccttgctcctgccgctggccttgctgctccacgccgccaggccggacatcgtgctgacccagagccccgccagcctggccgtgagcctgggccagagggccaccatcagctgcagggccagcaagagcgtgagcaccagcggctacagctacctgcactggtaccagcagaagcccggccagccccccaagctgctgatctacctggccagcaacctggagagcggcgtgcccgccaggttcagcggcagcggcagcggcaccgacttcaccctgaacatccaccccgtggaggaggaggacgccgccacctactactgccagcacagcagggagctgcccttcaccttcggcagcggcaccaagctggagatcaagggaggggggggatccgggggaggaggctccggcggaggcggaagccaggtgcagctggtggagagcggcggcggcctggtgcagcccggcggcagcctgaagctgagctgcgccgccagcggcttcgacttcagcaggtactggatgagctgggtgaggcaggcccccggcaagggcctggagtggatcggcgagatcaaccccaccagcagcaccatcaacttcacccccagcctgaaggacaaggtgttcatcagcagggacaacgccaagaacaccctgtacctgcagatgagcaaggtgaggagcgaggacaccgccctgtactactgcgccaggggcaactactacaggtacggcgacgccatggactactggggccagggcaccagcgtgaccgtgagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaagtttaaac

SEQ ID NO: 17 - protein, 491 aa, synthetic construct
  REGION 1..491: note = synthetic sequence
MALPVTALLLPLALLLHAARPDIVLTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIKGGGGSGGGGSGGGGSQVQLVESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 18 - DNA, 1492 bp, synthetic construct
  misc_feature 1..1492: note = synthetic sequence
  source 1..1492: mol_type = other DNA
gcgatcgcatggccttaccagtgaccgccttgctcctgccgctggccttgctgctccacgccgccaggccggacatcgtgctgacccagagccccgccagcctggccgtgagcctgggccagagggccaccatcagctgcagggccagcaagagcgtgagcaccagcggctacagctacctgcactggtaccagcagaagcccggccagccccccaagctgctgatctacctggccagcaacctggagagcggcgtgcccgccaggttcagcggcagcggcagcggcaccgacttcaccctgaacatccaccccgtggaggaggaggacgccgccacctactactgccagcacagcagggagctgcccttcaccttcggcagcggcaccaagctggagatcaagggaggggggggatccgggggaggaggctccggcggaggcggaagccaggtgcagctggtggagagcggcggcggcctggtgcagcccggcggcagcctgaagctgagctgcgccgccagcggcttcgacttcagcaggtactggatgagctgggtgaggcaggcccccggcaagggcctggagtggatcggcgagatcaaccccaccagcagcaccatcaacttcacccccagcctgaaggacaaggtgttcatcagcagggacaacgccaagaacaccctgtacctgcagatgagcaaggtgaggagcgaggacaccgccctgtactactgcgccaggggcaactactacaggtacggcgacgccatggactactggggccagggcaccagcgtgaccgtgagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaagtttaaac

SEQ ID NO: 19 - protein, 997 aa, synthetic construct
  REGION 1..997: note = synthetic sequence
MALPVTALLLPLALLLHAARPDVVMTQSHRFMSTSVGDRVSITCRASQDVNTAVSWYQQKPGQSPKLLIFSASYRYTGVPDRFTGSGSGADFTLTISSVQAEDLAVYYCQQHYSTPWTFGGGTKLEIKGGGGSGGGGSGGGGSQIQLVQSGPDLKKPGETVKLSCKASGYTFTNFGMNWVKQAPGKGFKWMAWINTTRYTGESYFADDFKGRFAFSVETSATTAYLQINNLKTEDTATYFCARGEIYYGYDGGFAYWGQGTLVTVSATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGATNFSLLKQAGDVEENPGPALPVTALLLPLALLLHAARPDIQMTQSPSSLSASVGDRVTITCKASQDVGIAVAWYQQKPGKVPKLLIYWASTRHTGVPDRFSGSGSGTDFTLTISSLQPEDVATYYCQQYSSYPYTFGQGTKVEIKGGGGSGGGGSGGGGSEVQLVESGGGLVQPGGSLRLSCAASGFDFSRYWMSWVRQAPGKGLEWIGEINPDSSTINYAPSLKDKFIISRDNAKNSLYLQMNSLRAEDTAVYYCARPDGNYWYFDVWGQGTLVTVSSTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 20 - DNA, 2994 bp, synthetic construct
  misc_feature 1..2994: note = synthetic sequence
  source 1..2994: mol_type = other DNA
atggccttaccagtgaccgccttgctcctgccgctggccttgctgctccacgccgccaggccggatgtggtgatgacccagagccatcgctttatgagcaccagcgtgggcgatcgcgtgagcattacctgccgcgcgagccaggatgtgaacaccgcggtgagctggtatcagcagaaaccgggccagagcccgaaactgctgatttttagcgcgagctatcgctataccggcgtgccggatcgctttaccggcagcggcagcggcgcggattttaccctgaccattagcagcgtgcaggcggaagatctggcggtgtattattgccagcagcattatagcaccccgtggacctttggcggcggcaccaaactggaaattaaaggaggggggggatccgggggaggaggctccggcggaggcggaagccagattcagctggtgcagagcggcccggatctgaaaaaaccgggcgaaaccgtgaaactgagctgcaaagcgagcggctatacctttaccaactttggcatgaactgggtgaaacaggcgccgggcaaaggctttaaatggatggcgtggattaacaccacccgctataccggcgaaagctattttgcggatgattttaaaggccgctttgcgtttagcgtggaaaccagcgcgaccaccgcgtatctgcagattaacaacctgaaaaccgaagataccgcgacctatttttgcgcgcgcggcgaaatttattatggctatgatggcggctttgcgtattggggccagggcaccctggtgaccgtgagcgcgaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggaagcggagccaccaacttcagcctgctgaagcaggccggcgacgtggaggagaaccccggccccgccttaccagtgaccgccttgctcctgccgctggccttgctgctccacgccgccaggccggatattcagatgacccagagcccgagcagcctgagcgcgagcgtgggcgatcgcgtgaccattacctgcaaagcgagccaggatgtgggcattgcggtggcgtggtatcagcagaaaccgggcaaagtgccgaaactgctgatttattgggcgagcacccgccataccggcgtgccggatcgctttagcggcagcggcagcggcaccgattttaccctgaccattagcagcctgcagccggaagatgtggcgacctattattgccagcagtatagcagctatccgtatacctttggccagggcaccaaagtggaaattaaaggaggggggggatccgggggaggaggctccggcggaggcggaagcgaagtgcagctggtggaaagcggcggcggcctggtgcagccgggcggcagcctgcgcctgagctgcgcggcgagcggctttgattttagccgctattggatgagctgggtgcgccaggcgccgggcaaaggcctggaatggattggcgaaattaacccggatagcagcaccattaactatgcgccgagcctgaaagataaatttattattagccgcgataacgcgaaaaacagcctgtatctgcagatgaacagcctgcgcgcggaagataccgcggtgtattattgcgcgcgcccggatggcaactattggtattttgatgtgtggggccagggcaccctggtgaccgtgagcagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 21 - protein, 997 aa, synthetic construct
  REGION 1..997: note = synthetic sequence
MALPVTALLLPLALLLHAARPDVVMTQSHRFMSTSVGDRVSITCRASQDVNTAVSWYQQKPGQSPKLLIFSASYRYTGVPDRFTGSGSGADFTLTISSVQAEDLAVYYCQQHYSTPWTFGGGTKLDIKGGGGSGGGGSGGGGSQIQLVQSGPDLKKPGETVKLSCKASGYTFTNFGMNWVKQAPGKGFKWMAWINTYTGESYFADDFKGRFAFSVETSATTAYLQINNLKTEDTATYFCARGEIYYGYDGGFAYWGQGTLVTVSATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGATNFSLLKQAGDVEENPGPMALPVTALLLPLALLLHAARPDIQMTQSPSSLSASVGDRVTITCKASQDVGIAVAWYQQKPGKVPKLLIYWASTRHTGVPDRFSGSGSGTDFTLTISSLQPEDVATYYCQQYSSYPYTFGQGTKVEIKGGGGSGGGGSGGGGSEVQLVESGGGLVQPGGSLRLSCAASGFDFSRYWMSWVRQAPGKGLEWIGEINPDSSTINYAPSLKDKFIISRDNAKNSLYLQMNSLRAEDTAVYYCARPDGNYWYFDVWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 22 - DNA, 3009 bp, synthetic construct
  misc_feature 1..3009: note = synthetic sequence
  source 1..3009: mol_type = other DNA
cgatcgcatggccttaccagtgaccgccttgctcctgccgctggccttgctgctccacgccgccaggccggatgtggtgatgacccagagccatcgctttatgagcaccagcgtgggagatcgagtgagcattacctgccgcgcgagccaggatgtgaacaccgcggtgagctggtatcagcagaaaccgggccagagcccgaaactgctgatttttagcgcgagctatcgctataccggcgtgccggatcgctttaccggcagcggcagcggcgcggattttaccctgaccattagcagcgtgcaggcggaagatctggcggtgtattattgccagcagcattatagcaccccgtggacctttggcggcggcaccaaactggatattaaaggaggggggggatccgggggaggaggctccggcggaggcggaagccagattcagctggtgcagagcggcccggatctgaaaaaaccgggcgaaaccgtgaaactgagctgcaaagcgagcggctatacctttaccaactttggcatgaactgggtgaaacaggcgccgggcaaaggctttaaatggatggcgtggattaacacctataccggcgaaagctattttgcggatgattttaaaggccgctttgcgtttagcgtggaaaccagcgcgaccaccgcgtatctgcagattaacaacctgaaaaccgaagataccgcgacctatttttgcgcgcgcggcgaaatttattatggctatgatggcggctttgcgtattggggccagggcaccctggtgaccgtgagcgcgaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaagcaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggaagcggagccaccaacttcagcctgctgaagcaggccggcgacgtggaggagaaccccggccccatggccttaccagtgaccgccttgctcctgccgctggccttgctgctccacgccgccaggccggatattcagatgacccagagcccgagcagcctgagcgcgagcgtgggcgaccgcgtgaccattacctgcaaagcgagccaggatgtgggcattgcggtggcgtggtatcagcagaaaccgggcaaagtgccgaaactgctgatttattgggcgagcacccgccataccggcgtgccggatcgctttagcggcagcggcagcggcaccgattttaccctgaccattagcagcctgcagccggaagatgtggcgacctattattgccagcagtatagcagctatccgtatacctttggccagggcaccaaagtggaaattaaaggaggggggggatccgggggaggaggctccggcggaggcggaagcgaagtgcagctggtggaaagcggcggcggcctggtgcagccgggcggcagcctgcgcctgagctgcgcggcgagcggctttgattttagccgctattggatgagctgggtgcgccaggcgccgggcaaaggcctggaatggattggcgaaattaacccggatagcagcaccattaactatgcgccgagcctgaaagataaatttattattagccgcgataacgcgaaaaacagcctgtatctgcagatgaacagcctgcgcgcggaagataccgcggtgtattattgcgcgcgcccggatggcaactattggtattttgatgtgtggggccagggcaccctggtgaccgtgagcagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaagcaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaagtttaaac

SEQ ID NO: 23 - DNA, 423 bp, synthetic construct
  misc_feature 1..423: note = synthetic sequence
  source 1..423: mol_type = other DNA
agctagctgcagtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccgggtacc

SEQ ID NO: 24 - protein, 165 aa, synthetic construct
  REGION 1..165: note = synthetic sequence
MSGLGRSRRGGRSRVDQEERFPQGLWTGVAMRSCPEEQYWDPLLGTCMSCKTICNHQSQRTCAAFCRSLSCRKEQGKFYDHLLRDCISCASICGQHPKQCAYFCENKLRSPVNLPPELRRQRSGEVENNSDNSGRYQGLEHRGSEASPALPGLKLSADQVALVYS

SEQ ID NO: 25 - protein, 54 aa, synthetic construct
  REGION 1..54: note = synthetic sequence
MLQMAGQCSQNEYFDSLLHACIPCQLRCSSNTPPLTCQRYCNASVTNSVKGTNA

SEQ ID NO: 26 - protein, 204 aa, synthetic construct
  REGION 1..204: note = synthetic sequence
SGPVKELVGSVGGAVTFPLKSKVKQVDSIVWTFNTTPLVTIQPEGGTIIVTQNRNRERVDFPDGGYSLKLSKLKKNDSGIYYVGIYSSSLQQPSTQEYVLHVYEHLSKPKVTMGLQSNKNGTCVTNLTCCMEHGEEDVIYTWKALGQAANESHNGSILPISWRWGESDMTFICVARNPVSRNFSSPILARKLCEGAADDPDSSM

SEQ ID NO: 27 - protein, 46 aa, synthetic construct
  REGION 1..46: note = synthetic sequence
MDDSTEREQSRLTSCLKKREEMKLKECVSILPRKESPSVRSSKDGK

SEQ ID NO: 28 - protein, 242 aa, synthetic construct
  REGION 1..242: note = synthetic sequence
DPNFWLQVQESVTVQEGLCVLVPCTFFHPIPYYDKNSPVHGYWFREGAIISRDSPVATNKLDQEVQEETQGRFRLLGDPSRNNCSLSIVDARRRDNGSYFFRMERGSTKYSYKSPQLSVHVTDLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSGKQETRAGVVH

SEQ ID NO: 29 - protein, 287 aa, synthetic construct
  REGION 1..287: note = synthetic sequence
TKEDPNPPITNLRMKAKAQQLTWDLNRNVTDIECVKDADYSMPAVNNSYCQFGAISLCEVTNYTVRVANPPFSTWILFPENSGKPWAGAENLTCWIHDVDFLSCSWAVGPGAPADVQYDLYLNVANRRQQYECLHYKTDAQGTRIGCRFDDISRLSSGSQSSHILVRGRSAAFGIPCTDKFVVFSQIEILTPPNMTAKCNKTHSFMHWKMRSHFNRKFRYELQIQKRMQPVITEQVRDRTSFQLLNPGTYTVQIRARERVYEFLSAWSTPQRFECDQEEGANTRAWR

SEQ ID NO: 30 - protein, 272 aa, synthetic construct
  REGION 1..272: note = synthetic sequence
PEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWK

SEQ ID NO: 31 - protein, 6 aa, synthetic construct
  REGION 1..6: note = synthetic sequence
PICVTV

SEQ ID NO: 32 - protein, 47 aa, synthetic construct
  REGION 1..47: note = synthetic sequence
KISHFLKMESLNFIRAHTPYINIYNCEPANPSEKNSPSTQYCYSIQS

SEQ ID NO: 33 - protein, 480 aa, synthetic construct
  REGION 1..480: note = synthetic sequence
DSSKWVFEHPETLYAWEGACVWIPCTYRALDGDLESFILFHNPEYNKNTSKFDGTRLYESTKDGKVPSEQKRVQFLGDKNKNCTLSIHPVHLNDSGQLGLRMESKTEKWMERIHLNVSERPFPPHIQLPPEIQESQEVTLTCLLNFSCYGYPIQLQWLLEGVPMRQAAVTSTSLTIKSVFTRSELKFSPQWSHHGKIVTCQLQDADGKFLSNDTVQLNVKHTPKLEIKVTPSDAIVREGDSVTMTCEVSSSNPEYTTVSWLKDGTSLKKQNTFTLNLREVTKDQSGKYCCQVSNDVGPGRSEEVFLQVQYAPEPSTVQILHSPAVEGSQVEFLCMSLANPLPTNYTWYHNGKEMQGRTEEKVHIPKILPWHAGTYSCVAENILGTGQRGPGAELDVQYPPKKVTTVIQNPMPIREGDTVTLSCNYNSSNPSVTRYEWKPHGAWEEPSLGVLKIQNVGWDNTTIACAACNSWCSWASPVAL

SEQ ID NO: 34 - protein, 480 aa, synthetic construct
  REGION 1..480: note = synthetic sequence
QSPTPSPTGLTTAKMPSVPLSSDPLPTHTTAFSPASTFERENDFSETTTSLSPDNTSTQVSPDSLDNASAFNTTGVSSVQTPHLPTHADSQTPSAGTDTQTFSGSAANAKLNPTPGSNAISDVPGERSTASTFPTDPVSPLTTTLSLAHHSSAALPARTSNTTITANTSDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKC

SEQ ID NO: 35 - protein, 65 aa, synthetic construct
  REGION 1..65: note = synthetic sequence
ITCPPPMSVEHADIWVKSYSLYSRERYICNSGFKRKAGTSSLTECVLNKATNVAHWTTPSLKCIR

SEQ ID NO: 36 - protein, 153 aa, synthetic construct
  REGION 1..153: note = synthetic sequence
MYRMQLLSCIALSLALVTNSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 37 - protein, 24 aa, synthetic construct
  REGION 1..24: note = synthetic sequence
NSELLSLINDMPITNDQKKLMSNN

SEQ ID NO: 38 - protein, 17 aa, synthetic construct
  REGION 1..17: note = synthetic sequence
GTGGTGTGAGTAGGTAA

SEQ ID NO: 39 - protein, 17 aa, synthetic construct
  REGION 1..17: note = synthetic sequence
GAGTTTTGCATTGGCGG

SEQ ID NO: 40 - protein, 17 aa, synthetic construct
  REGION 1..17: note = synthetic sequence
GAGGGTGGTTGTCAATG

SEQ ID NO: 41 - DNA, 17 bp, synthetic construct
  misc_feature 1..17: note = synthetic sequence
  source 1..17: mol_type = other DNA
gtggtgtgagtaggtaa

SEQ ID NO: 42 - DNA, 17 bp, synthetic construct
  misc_feature 1..17: note = synthetic sequence
  source 1..17: mol_type = other DNA
gagttttgcattggcgg

SEQ ID NO: 43 - DNA, 17 bp, synthetic construct
  misc_feature 1..17: note = synthetic sequence
  source 1..17: mol_type = other DNA
gagggtggttgtcaatg

(19) United States
(12) Patent Application Publication
(10) Pub. No.: US 2023/0277622 A1
(43) Pub. Date: Sep. 7, 2023

(54) CHIMERIC ANTIGEN RECEPTORS (CARs) COMPOSITIONS AND METHODS THEREOF

(71) Applicant: iCell Gene Therapeutics Inc., Stony Brook, NY (US)

(72) Inventors: Yupo MA, Stony Brook, NY (US); Kevin PINZ, Stony Brook, NY (US); Masayuki WADA, Stony Brook, NY (US)

(21) Appl. No.: 17/994,787

(22) Filed: Nov. 28, 2022

Related U.S. Application Data
(63) Continuation-in-part of application No. 15/739,596, filed on Dec. 22, 2017, now Pat. No. 11,655,452.
(60) Provisional application No. 62/244,435, filed on Oct. 21, 2015.
(60) Provisional application No. 62/235,840, filed on Oct. 1, 2015.
(60) Provisional application No. 62/184,321, filed on Jun. 25, 2015.

(51) Int. Cl. A61K 38/17; A61K 39/00; A61K 38/20; A61P 35/00; A61P 37/00

(52) U.S. Cl. CPC A61K 38/1774; A61K 38/2086; A61K 39/4611; A61K 39/4631; A61P 35/00; A61P 37/00

(57) ABSTRACT

The present invention relates to compositions and methods relating to chimeric antigen receptor (CAR) polypeptides and methods relating thereto. In one embodiment, the present invention relates to engineered cells having chimeric antigen receptor polypeptides directed to at least two targets. In another embodiment, the present invention relates to engineered cells having chimeric antigen receptor polypeptides and an enhancer moiety.

### CROSS REFERENCE TO RELATED APPLICATIONS

This application is a continuation-in-part application of Application No. 15/739,596, filed Dec. 22, 2017, which is a national stage filing under 35 USC §371 of international application number PCT/US2016/039306, filed on Jun. 24, 2016, which claims the benefit of U.S. Provisional Application No. 62/184,321, filed on Jun. 25, 2015, U.S. Provisional Application No. 62/235,840, filed on Oct. 01, 2015, and U.S. Provisional Application No. 62/244,435, filed on Oct. 21, 2015. All of which are incorporated by reference herein in their entirety.

### BACKGROUND

T cells, a type of lymphocyte, play a central role in cell-mediated immunity. They are distinguished from other lymphocytes, such as B cells and natural killer cells (NK cells), by the presence of a T-cell receptor (TCR) on the cell surface. T helper cells, also called CD4+ T or CD4 T cells, express CD4 glycoprotein on their surface. Helper T cells are activated when exposed to peptide antigens presented by MHC (major histocompatibility complex) class II molecules. Once activated, these cells proliferate rapidly and secrete cytokines that regulate immune response. Cytotoxic T cells, also known as CD8+ T cells or CD8 T cells, express CD8 glycoprotein on the cell surface. The CD8+ T cells are activated when exposed to peptide antigens presented by MHC class I molecules. Memory T cells, a subset of T cells, persist long term and respond to their cognate antigen, thus providing the immune system with “memory” against past infections and/or tumor cells.

T cells can be genetically engineered to produce special receptors on their surface called chimeric antigen receptors (CARs). CARs are proteins that allow the T cells to recognize a specific protein (antigen) on tumor cells. These engineered CAR T cells are then grown in the laboratory until they number in the billions. The expanded population of CAR T cells is then infused into the patient.

Clinical trials to date have shown chimeric antigen receptor (CAR) T cells to have great promise in hematologic malignancies resistant to standard chemotherapies. Most notably, CD19-specific CAR (CD19CAR) T-cell therapies have had remarkable results including long-term remissions in B-cell malignancies (Kochenderfer, Wilson et al. 2010, Kalos, Levine et al. 2011, Porter, Levine et al. 2011, Davila, Riviere et al. 2013, Grupp, Frey et al. 2013, Grupp, Kalos et al. 2013, Kalos, Nazimuddin et al. 2013, Kochenderfer, Dudley et al. 2013, Kochenderfer, Dudley et al. 2013, Lee, Shah et al. 2013, Park, Riviere et al. 2013, Maude, Frey et al. 2014).

Despite the success of CAR therapy in B-cell leukemia and lymphoma, the application of CAR therapy to T-cell malignancies has not yet been well established. Given that T-cell malignancies are associated with dramatically poorer outcomes compared to those of B-cell malignancies (Abramson, Feldman et al. 2014), CAR therapy in this respect has the potential to further address a great clinical need.

To date, current efforts have focused on CAR T-cells demonstrating efficacy in various B-cell malignancies. While initial remission rates of approximately 90% are common in B-ALL using CD19CAR, most of these relapse within a year. The relapse is at least in part due to the antigen escape. Thus, more effective CAR T cell treatments in order to prevent the relapse is urgently needed. Target discovery and selection are the initial step as there are no general rules to ensure or guide CAR design that are efficacious.

There are some roadblocks that hinder the broader adoption of CAR therapeutic approach. Among the most general challenges are: (1) selection of antigen target and chimeric antigen receptor(s); (2)CAR design; (3)tumor heterogeneity, particularly the variance in the surface expression of tumor antigens. Targeting single antigen carries the risk of immune escape and this could be overcome by targeting multiple desired antigens.

Most CAR chimeric antigen receptors are scFvs derived from monoclonal antibodies and some of these monoclonal antibodies have been used in the clinical trials or treatment for diseases. However, they have limited efficacy, which suggests that alternative and more potent targeting approaches, such as CARs are required. scFvs are the most commonly used chimeric antigen receptor for CARs. However, CAR affinity binding and locations of the recognized epitope on the antigen could affect the function. Additionally the level of the surface CAR expression on the T cells or NK cells is affected by an appropriate leader sequence and promoter. Furthermore, overexpressed CAR proteins can be toxic to cells.

Therefore, there remains a need for improved chimeric antigen receptor-based therapies that allow for more effective, safe, and efficient targeting of T-cell associated malignancies.

### SUMMARY OF THE INVENTION

In one embodiment, the present disclosure provides an engineered cell having a first chimeric antigen receptor polypeptide including a first antigen recognition domain, a first signal peptide, a first hinge region, a first transmembrane domain, a first co-stimulatory domain, and a first signaling domain; and a second chimeric antigen receptor polypeptide including a second antigen recognition domain, a second signal peptide, a second hinge region, a second transmembrane domain, a second co-stimulatory domain, and a second signaling domain; wherein the first antigen recognition domain is different than the second antigen recognition domain.

In another embodiment, the present disclosure provides an engineered polypeptide including a chimeric antigen receptor and an enhancer.

In another embodiment, the present disclosure provides an engineered polypeptide including a chimeric antigen receptor polypeptide and an enhancer.

In another embodiment, the present disclosure provides an engineered chimeric antigen receptor polypeptide, the polypeptide including: a signal peptide, a CD45 antigen recognition domain, a hinge region, a transmembrane domain, at least one co-stimulatory domain, and a signaling domain. In another embodiment, the present disclosure provides a polynucleotide encoding for the aforementioned polypeptide.

In another embodiment, the present disclosure provides an engineered cell having the engineered polypeptide or polynucleotide described above.

In another embodiment, the present disclosure provides a method of reducing the number of target cells including the steps of (i.) contacting said target cells with an effective amount of an engineered cell having at least one chimeric antigen receptor polypeptide, for engineered cells having multiple chimeric antigen receptor polypeptides, each chimeric antigen receptor polypeptides are independent; and (ii.) optionally, assaying for the reduction in the number of said cells. The target cells include at least one cell surface antigen selected from the group consisting of interleukin 6 receptor, NY-ESO-1, alpha fetoprotein (AFP), glypican-3 (GPC3), BAFF-R, BCMA, TACI, LeY, CD5, CD13, CD14, CD15 CD19, CD20, CD22, CD33, CD41, CD45, CD61, CD64, CD68, CD117, CD123, CD138, CD267, CD269, CD38, Flt3 receptor, and CS1.

In another embodiment, the present disclosure provides methods for treating B-cell lymphoma, T-cell lymphoma, multiple myeloma, chronic myeloid leukemia, B-cell acute lymphoblastic leukemia (B-ALL), and cell proliferative diseases by administering any of the engineered cells described above to a patient in need thereof.

### BRIEF DESCRIPTION OF DRAWINGS

The patent or patent application contains at least one drawing executed in color. Copies of the patent or patent application publication with color drawing(s) will be provided by the United States Patent and Trademark Office upon request and payment of the necessary fee.

FIG. 1. A schematic representation of cCAR construct (hereinafter, “multiple CAR or compound CAR”). Multiple or compound CAR targets multiple antigens (e.g. cell type 1 or cell type 2 or the same cell type). Multiple or cCAR T cell immunotherapies comprises individual component CAR comprising a different or same antigen recognition domain, a hinge region, a transmembrane domain, various co-stimulatory domain(s) and an intracellular signaling domain.

FIG. 2A. A schematic representation of cCAR-T construct. The construct comprises a SFFV promoter driving the expression of-multiple modular units of CARs linked by a P2A peptide. Upon cleavage of the linker, the cCARs split and engage upon targets expressing CD33 and/or CD123. As a novel cCAR construct, the activation domains of the construct may include, but is not limited to, 4-1BB on the CD33 CAR segment and a CD28 region on the CD123 CAR.

FIG. 2B. A Western blot depicting the expression of transduced CD33CD123 cCAR-T cells. The figure depicts expression of two different CAR proteins, i.e., CD33 CAR and CD123 CARs. The cCAR-T cells expressing both CD33 and CD123 CARs upon cleavage of the linker generate two distinct and consistently intense protein bands. Green Fluroscent Protein (GFP) is included as negative control.

FIG. 2C. Flow cytometry representing the efficiency of transduction. Upper panel shows the lentiviral titer for CD33CD123 cCARs (also referred to as CD33CD123-2G-CAR) tested on 293FT HEK (human embryonic kidney) cells to gauge maximum transduction efficiency before usage on UCB (umbilical cord blood) and PB (peripheral blood) T-cells. Lower panel shows CD33CD123 cCAR (also referred to as CD33CD123-2G-CAR) T-cells transduced with lentiviral vectors comprising CD33CD123 cCAR construct and GFP-transduced cells as control Percentages indicated by yellow circles are proxies for transduction efficiency.

FIG. 3. Schematic showing a method of generating a high-efficiency compound CAR (cCAR).

FIG. 4. A co-culture assay representing the incubation of CD33CD123-2G CAR-T cells (cCAR) with the promyelocytic leukemia cell line HL60. cCAR-T cell (lower panel) is compared to control GFP transduced T-cell (upper panel). The efficacy of the killing is measured by the population of CD33+ cells that is left over after incubation for about 24 hours (enclosed in yellow circles).

FIG. 5. A co-culture assay representing incubation of cCAR-T cells with the myelogenous leukemia cell line KG-1a, which expresses about 100% CD33 and about 50-80% CD123. cCAR-T cell (lower panel) is compared to control GFP transduced T-cell (upper panel). The efficacy of the killing is measured by the population of CD33+ cells that is left over after incubation for about 24 hours.

FIG. 6. A co-culture assay representing incubation of cCAR-T cells with AML patient samples (here referred to as AML-9). The patient cells include mixed populations of cells, such as for example, leukemia cells, monocytes, and other types of blasts. CD33 acts as a marker for CAR-T action as well as CD34, a specific marker for leukemia cells. The CAR-T panel (right) is compared to control GFP transduced T-cells (middle). The efficacy of the killing is measured by the population of CD33+/CD34+ cells that is left over after incubation for at least 24 hours.

FIG. 7. A co-culture assay representing incubation of cCAR-T cells with B-ALL patient samples (here referred to as Sp-BM-B6). The patient cells include mixed populations of cells, such as, for example, leukemia cells, monocytes, and other types of blasts. CD34 acts as a specific marker for leukemia cells. The CAR-T panel (right) is compared to control GFP transduced T-cells (middle). The efficacy of the killing is measured by the population of CD34+ cells left over after incubation for at least 24 hours.

FIG. 8. CD33CD123 cCAR expression in NK-92 cells. The CD33CD123 cCAR expression are detected using goat-anti-mouse antibody, F(ab)2.

FIG. 9. A co-culture assay representing incubation of cCAR NK-92 cells with HL-60. The cCAR NK-92 cells are compared with GFP transduced NK-92 cells. The efficacy of the killing is measured by the population of CD33+ cells left over after incubation for about 24 hours.

FIG. 10. A co-culture assay representing incubation of cCAR NK-92 cells with KG1a. The cCAR NK cell panel is compared with GFP transduced NK-92 cells. The efficacy of the killing is measured by the population of CD33+ cells left over after incubation for about 24 hours.

FIG. 11. Dose response of CD33CD123 cCAR (CAR-CD33/123) NK-92 cells with HL-60 or KG1a.

The efficacy of the killing is measured by the population of CD33+ cells left over after incubation for about 24 hours.

FIG. 12. A comparison of CD33CD123 cCAR NK-92 cell killing ability with control in two populations of KG11 cells. Assays were performed at different ratios of CAR-CD33/123 (CD33CD123 cCAR NK-92 cells) and target cells, kG1a. The efficacy of the killing is measured by the population of CD33+CD123+ or CD33+CD123- cells left over after incubation for about 24 hours.

FIG. 13. A schematic representation of cCAR. The construct comprises a SFFV promoter driving the expression of multiple modular units of CARs linked by a linker. Upon cleavage of the linker, the cCARs split and engage upon targets expressing combinations of various target antigens: CD19 and/or CD20, and/or CD22 and/or 138. Multiple cCARs utilize the same or different co-stimulatory domains, such as, without limiting 4-1BB (also labeled as 4-BB) and/or CD28.

FIGS. 14A-C. BCMA-CS1 cCAR construct scheme (BC1cCAR). FIG. 14A: The construct consists a SFFV promoter driving the expression of two modular units of CAR linked by a P2A peptide. Upon cleavage of this P2A peptide, the cCARs split and engage upon targets expressing BCMA and /or CS1. Two unit CARs use same co-stimulatory domain, 4-1BB. Figured 14B: Flow cytometry analysis of BC1cCAR expression on T cell surface for vector (left) and BC1cCAR (right, highlighted by a square) showing 15.3% positive for F(Ab)2. Gating done against isotype controls. FIG. 14C: Preliminary functional validation of BC1cCAR-T cells by co-culturing K562 cells transduced with BCMA cDNA (BCMA-K562) (obtained from Kochenderfer, NIH). Bar graph shows lysis of the BCMA-K562 cell line vs. control T-cells as well as lysis of wild-type K562 (wt-K562) vs. control.

FIG. 14D. BCMA-CS1-2G construct using two different co-stimulatory domains either 4-1BB or CD28 for each unit. The construct includes a SFFV promoter driving the expression of two modular units of CAR linked by a P2A peptide. Upon cleavage of this P2A peptide, the cCARs split and engage targets expressing BCMA and /or CS1. Two unit CARs use a different co-stimulatory domain, either 4-1BB or CD28. Flow cytometry analysis of BC1cCAR expression on T cell surface for vector (left) and BC1cCAR (right, highlighted by a square) showing rare positive cells for F(Ab)2. Gating done against isotype controls.

FIG. 14E. Protein expression of BC1cCAR and BCMA-CS1-2G in HEK-293FT cells. HEK-293FT cells were transfected with lentiviral plasmids for GFP (lane 1), BC1cCAR (lane 2), CD269-CS 1-2G (lane 3) 48 hours after transfection, supernatant was removed, and cells were also removed. Cells were lysed for Western blot and probe with mouse anti-human CD3z antibody.

FIGS. 15A-B. MM1S cell line co-culture. Co-cultures were carried out under 24 hours and collected and analyzed via flow cytometry. Target MM1S cells (myeloma cells) were labeled with Cytotracker (CMTMR) dye to distinguish it from effector T-cells. Populations were gated by anti-BCMA (CD269) and anti-CS1 (CD319) antibodies. FIG. 15A: Flow cytometry depictions of co-cultures. FIG. 15B: right: graphical summary of lysis vs. E:T ratio.

FIGS. 16A-B. RPMI-8226 cell line co-culture. Co-cultures were carried out under 24 hours and collected and analyzed via flow cytometry. Target RPMI-8226 cells were labeled with Cytotracker (CMTMR) dye to distinguish it from effector T-cells. Populations were gated by anti-BCMA (CD269) and anti-CS1 (CD319) antibodies. FIG. 16A: flow cytometry depictions of co-cultures. FIG. 16B: graphical summary of lysis vs. E:T ratio.

FIGS. 17A-B. U266 cell line co-culture. Co-cultures were carried out under 24 hours and collected, and analyzed via flow cytometry. Target U266 cells were labeled with Cytotracker (CMTMR) dye to distinguish it from effector T-cells. Populations were gated by anti-BCMA (CD269) and anti-CS 1 (CD319) antibodies. FIG. 17A: flow cytometry depictions of co-cultures. Figured 17B: graphical summary of lysis vs. E:T ratio.

FIGS. 18A-B. MM10-G primary patient sample co-culture and specific lysis. Co-cultures were carried out under 24 hours and collected and analyzed via flow cytometry. Target MM10-G cells were labeled with Cytotracker (CMTMR) dye to distinguish it from effector T-cells. Populations were gated by anti-BCMA (CD269) and anti-CS1 (CD319) antibodies. Notably, gating shows MM10-G presenting with distinct BCMA⁺ and CS1⁺ populations. FIG. 18A: flow cytometry depictions of co-cultures. FIG. 18B: graphical summary of lysis vs. E:T ratio.

FIGS. 19A-B. MM7-G primary patient sample co-culture and specific lysis. Co-cultures were carried out under 24 hours and collected and analyzed via flow cytometry. Target MM7-G cells were labeled with Cytotracker (CMTMR) dye to distinguish it from effector T-cells. Populations were gated by anti-BCMA (CD269) and anti-CS1 (CD319) antibodies. FIG. 19A: flow cytometry depictions of co-cultures. FIG. 19B: graphical summary of lysis vs. E:T ratio.

FIGS. 20A-B. MM11-G primary patient sample co-culture and specific lysis. Co-cultures were carried out under 24 hours and collected and analyzed via flow cytometry. Target MM11-G cells were labeled with Cytotracker (CMTMR) dye to distinguish it from effector T-cells. Populations were gated by anti-BCMA (CD269) and anti-CS1 (CD319) antibodies. FIG. 20A: flow cytometry depictions of co-cultures. FIG. 20B: graphical summary of lysis vs. E:T ratio.

FIG. 21. CD269-CS1-BBCAR NK cells demonstrate anti-leukemic effects in vivo. NSG mice were sublethally irradiated and intravenously injected the following day with luciferase-expressing MM.1S multiple myeloma cells to induce measurable tumor formation. After 3 days, the mice were intravenously injected with 8 × 10⁶ CD269-CS1-BBCAR NK cells or vector control NK control cells. On days 3, 6, and 8, mice were injected subcutaneously with RediJect D-Luciferin and subjected to IVIS imaging. Average light intensity measured for the CD269-CS 1-BBCAR NK injected mice was compared to that of vector control NK injected mice.

FIG. 22. Percent survival of mice was measured and compared between the two groups based on the studies from FIG. 21.

FIG. 23. CRISPR/Cas9 interference system. The expression of sgRNA and Cas9 puromycin is driven by the U6 and SFFV promoters, respectively. The Cas9 is linked with puromycin resistant gene by E2A self-cleaving sequences.

FIG. 24. A schematic providing an example of the steps for generation of CAR T or NK cell targeting hematologic malignancies.

FIG. 25. Generation and cell sorting of stable CD45 knockdown NK-92 cells using CRISPR/Cas9 lentivirus system. Flow cytometry analysis indicated the CD45 expression levels on NK-92 cell surface (left panels). After transduction of sgCD45B CRISPR into NK-92 cells, transduced cells were cultured in medium containing puromycin for a few weeks. CD45 negative NK-92 cells were determined using CD45 antibody and were sorted. The purity of stable NK⁴⁵ⁱ-92 (CD45 knockdown) NK-92 cells were determined by Flow cytometry analysis (right panel). This data showed that NK⁴⁵ⁱ-92 cells were successfully generated and obtained.

FIG. 26. Cell growth curve of wild type, GFP transduced NK-92 or NK⁴⁵ⁱ-92NK cells. To evaluate the effect for cell proliferation caused by CD45-knockdown (KD) in NK-92 cells, the number of cells of NK-92(•), GFP-transduced NK-92(■) and NK⁴⁵ⁱ-92(▲) were counted at 48 h and 96 h after seeding into 24 well plates. IL-2 was added at 48 h time point. (n=3 independent experiments performed in duplicate). Data are mean ± S.D. These data indicated that knockdown of CD45 receptor on NK-92 show similar cell growth curve compared to non-transduced NK-92 or GFP-transduced NK-92 cells.

FIGS. 27A-B. Co-culture assay with CCRF-CEM (target: T) and GFP NK-92 or GFP NK⁴⁵ⁱ-92 cells (effector: E), 5:1 (E:T) ratio. 16 hours incubation. FIG. 27A: Flow cytometry analysis of CCRF-CEM only (blue dot in left panel), in co-culture with CCRF-CEM and control GFP transduced NK-92 cells (middle panel) or GFP NK⁴⁵ⁱ-92 cells (right panel). Blue dots in all of panels indicates the leftover target CCRF-CEM cells and red dots shows effector cells by co-culture assay. All of incubation time were 16 h and the ratio of effector T-cells: target cell was 5:1. All experiments were performed in duplicate. FIG. 27B: Bar graph indicates the percent of cell lysis by the GFP transduced NK⁴⁵ⁱ-92 cells compared to the control GFP transduced NK92 cells in co-culture assay with CCRF-CEM. These data suggest that knockdown of CD45 in NK-92 cells does not show a significant difference for killing activity against CCRF-CEM cells compared to GFP-control NK-92 cells in vitro co-culture assay. Blue dotes are in the upper left quadrant.

FIGS. 28A-B. Co-culture assay with CCRF-CEM (target: T) and GFP NK-92, CD5CAR NK-92 or CD5CAR NK⁴⁵ⁱ-92 cells (effector: E). 5:1 (E:T) ratio. 16 hours incubation FIG. 28A: Flow cytometry analysis of CCRF-CEM only (left panel), in co-culture with CCRF-CEM and control GFP NK-92 cells (middle left panel), CD5CAR NK-92 cells (middle right panel), CD5CAR NK⁴⁵ⁱ-92 cells (right panel) from right to left. Blue dots in all of panels indicates the leftover target CCRF-CEM cells and red dots shows effector cells by co-culture assay. All of incubation times were 16 h and the ratio of effector T-cells: target cell is 5:1. All experiments were performed in duplicate. FIG. 28B: Bar graph indicates the percent of cell lysis by the CD5CAR NK-92 cells or CD5CAR NK⁴⁵ⁱ-92 cells compared to the control GFP NK92 cells in co-culture assay with CCRF-CEM. Data are mean ± S.D. Both of CD5CAR NK-cells and CD5CAR NK⁴⁵ⁱ-92 cells shows near to 100% cell killing activity against CD5-potitive CCRF-CEM compared to control GFP NK-92 cells. These data suggest that CD5CAR NK-cells and CD5CAR NK⁴⁵ⁱ-92 cells can effectively lyse CCRF-CEM cells that express CD5 compared to GFP-control NK-92 cells in vitro co-culture assay, and provide proof that knockdown of CD45 does not affect cell function for killing activity in NK-92 cells. Blue dots are in the upper left quadrant of the first two panels starting from the left.

FIGS. 29A-B. Organization of the CD45CAR construct and its expression. FIG. 29A: Schematic representation of the CD45CAR lentiviral vector. The CD45CAR construct is a modularized signaling domain containing: a leader sequence, an anti-CD45scFv, a hinge domain (H), a transmembrane domain (TM), two co-stimulatory domains (CD28 and 4-1BB) that define the construct as a 3^{rd} generation CAR, and the intracellular signaling domain CD3 zeta. FIG. 29B: HEK-293FT cells were transfected with lentiviral plasmids for GFP (lane 1) and CD45CAR (lane 2). 48 hours after transfection, supernatant was removed, and cells were also removed. Cells were lysed for Western blot and probe with mouse anti-human CD3z antibody.

FIGS. 30A-B. Transduction of CD45CAR into NK⁴⁵ⁱ-92 cells and cell sorting of CD45CAR transduced cells. FIG. 30A: The expression levels of CD45CAR on NK⁴⁵ⁱ-92 were determined by flow cytometry analysis (circled in blue at middle panel) compared to NK⁴⁵ⁱ-92 cells (left panel) after CD45CAR lentviruses were transduced into NK⁴⁵ⁱ-92 cells. CD45CAR expressed NK⁴⁵ⁱ-92 cells were sorted and CD45 expression levels on cell surface were determined by Flow cytometry analysis (right panel). FIG. 30B: About 87% of CD45CAR expression on cell surface was detected by flow cytometry analysis.

FIGS. 31A-B. Co-culture assay with CCRF-CEM (target: T) and GFP NK-92 or CD45CAR NK⁴⁵ⁱ-92 cells (effector: E). 5:1 (E:T) ratio. 16 hours incubation. FIG. 31A: Flow cytometry analysis of in co-culture with CCRF-CEM and control GFP transduced NK-92 cells (left panel) or CD45CAR NK⁴⁵ⁱ-92 cells (right panel). Blue dots in all of panels indicates the leftover target CCRF-CEM cells and red dots shows effector NK-92 cells by co-culture assay. All of incubation times were 16 h and the ratio of effector T-cells: target cell is 5:1. All experiments were performed in duplicate. FIG. 31B: Bar graph indicates the percent of cell lysis by CD45CAR NK⁴⁵ⁱ-92 cells compared to the control GFP NK92 cells in co-culture assay with CCRF-CEM. Data are mean ± S.D. CD45CAR NK⁴⁵ⁱ-92 cells shows about 70% cell lysis against CCRF-CEM cells compared to control GFP NK-92 cells. These data suggest that CD45CAR NK⁴⁵ⁱ-92 cells effectively lyse CCRF-CEM cells that express CD45 compared to GFP-control NK-92 cells in vitro co-culture assay.

FIGS. 32A-C. Co-culture assay with Jurkat cells (target: T) and GFP-control or CD45CAR NK⁴⁵ⁱ-92 cells (effector: E). 5:1 or 2:1 (E:T) ratio. 6 hours incubation. FIG. 32A: Flow cytometry analysis was carried out after Jurkat cells were stained by CMTMR cell tracker dye. These data shows that Jurkat cells are CD45 positive (left panels) and mostly CD56 negative cells (right panel). FIG. 32B: Flow cytometry analysis of co-culture assay with Jurkat cells (target: T) and control or CD45CAR NK⁴⁵ⁱ-92 cells (effector: E). The ratio of co-culture assay was performed in 5:1 or 2:1 (E: T). Left panels showed that in co-culture with control GFP or CD45CAR/CD45KD NK-92 cells in 5:1 (E:T) ratio and right panels indicated that in co-culture with control GFP or CD45CAR NK⁴⁵ⁱ-92 cells in 2:1 (E:T) ratio. Blue dots in panels indicate the leftover target Jurkat cells and red dots represent effector cells by co-culture assay. All of incubation time were 6 h. All experiments were performed in duplicate. FIG. 32C: Bar graph shows percent cell lysis by CD45CAR NK⁴⁵ⁱ-92 cells compared to control GFP NK92 cells at in 5:1 or 2:1 (E: T) ratio. Data are mean ± S.D. CD45CAR NK⁴⁵ⁱ-92 cells shows about 60% cell lysis against Jurkat cells compared to control GFP NK-92 cells in both conditions. This data suggests that CD45CAR NK⁴⁵ⁱ-92 cells effectively lyse Jurkat cells that express CD45 on cell surface compared to GFP-control NK-92 cells in vitro co-culture assay.

FIGS. 33A-C. Co-culture assay with GFP-NK-92 cells (target: T) and non-transduced NK-92 cells or CD45CAR NK⁴⁵ⁱ-92 cells (effector: E). 5:1 or 2:1 (E:T) ratio. 6 hours incubation. FIG. 33A: Flow cytometry analysis was carried out using GFP control NK-92 cells. These data proof that GFP control NK-92 cells are about 99% GFP positive cells (green dots). FIG. 33B: Flow cytometry analysis of co-culture assay with GFP control NK-92 cells (target: T) and non-transduced or CD45CAR NK⁴⁵ⁱ-92 cells (effector: E). The ratio of co-culture assay was performed in 5:1 or 2:1 (E: T). Left panels showed that in co-culture with non-transduced or CD45CAR NK⁴⁵ⁱ-92 cells in 5:1 (E:T) ratio and right panels indicated that in co-culture with non-transduced or CD45CAR NK⁴⁵ⁱ-92 cells in 2:1 (E:T) ratio. Green dots in panels indicate the leftover target GFP NK-92 cells and red dots represent effector cells by co-culture assay. The incubation time was 6 h. All experiments were performed in duplicate. FIG. 33C: Bar graph shows percent cell lysis of GFP NK-92 cells by CD45CAR NK⁴⁵ⁱ-92 cells compared to non-transduced NK-92 cells at in 5:1 or 2:1 (E: T) ratio. Data are mean ± S.D. CD45CAR NK⁴⁵ⁱ-92 cells shows about 20% cell lysis in 2:1 (E:T) ratio and about 55% cell lysis in 5:1 (E:T) ratio against GFP NK-92 cells compared to non-transduced NK-92 cells. This data suggests that CD45CAR NK⁴⁵ⁱ-92 cells effectively lyse GFP NK-92 cells that express CD45 on cell surface compared to non-transduced NK-92 cells in vitro co-culture assay. Green dots are in the upper right quadrant of each panel.

FIGS. 33D-E. Transduction of CD45b-BB or CD45b-28 into NK⁴⁵ⁱ-92 cells and cell sorting of CD45b-BB or CD45b-28 transduced NK⁴⁵ⁱ-92 cells. FIG. 33D: The surface expression levels of CD45b-BB CAR or CD45b-28 CAR on NK⁴⁵ⁱ-92 were determined by flow cytometry analysis (circled in blue at middle panel) compared to NK⁴⁵ⁱ-92 cells (left panel) after CD45b-BB or CD45b-28 lentviruses transduced into NK45i-92 cells. FIG. 33E: NK45i-92 cells expressing the CD45b-BB or CD45b-28 CAR were sorted by Flow cytometry analysis. About 74% of CD45b-BB CAR or 82% of CD45b-28 CAR expression on cell surface was detected by flow cytometry analysis.

FIGS. 33F-G. Co-culture assay with REH cells (target: T) and GFP NK-92 cells or CD45CAR NK⁴⁵ⁱ-92 cells or CD45b-BB NK⁴⁵ⁱ-92 cells or CD45b-28 NK⁴⁵ⁱ-92 cells (effector: E). 5:1 (E:T) ratio. 20 hours incubation. FIG. 33F: Flow cytometry analysis of REH cells only (left panel), in co-culture with REH cells and control GFP transduced NK-92 cells (2nd left panel), CD45CAR NK⁴⁵ⁱ-92 cells (middle panel), CD45b-BB NK⁴⁵ⁱ-92 cells (4th from left panel) or CD45b-28 NK⁴⁵ⁱ-92 cells (right panel). Blue dots in all of panels indicate the leftover target REH cells and red dots shows effector GFP or CARs-NK-92 cells by co-culture assay. REH is a B acute lymphoblastic cell line. All of incubation times were 20h and the ratio of effector NK-cells: target cell is 5:1. All experiments were performed in duplicate. FIG. 33G: Bar graph indicates the percent of cell lysis by CD45CAR NK⁴⁵ⁱ-92 cells, CD45b-BB NK⁴⁵ⁱ-92 cells or CD45b-28 NK45i-92 cells compared to the control GFP NK92 cells in co-culture assay with REH cells. Data are mean + S.D. CD45CAR NK⁴⁵ⁱ-92 cells shows about 76% cell lysis, CD45b-BB NK⁴⁵ⁱ-92 cells shows about 79% cell lysis and CD45b-28 NK⁴⁵ⁱ-92 shows 100% cell lysis against REH cells compared to control GFP NK-92 cells. These data suggest that all three CD45CARs effectively lyse REH cells.

FIGS. 34A-B. Schematic diagram to elucidate the construct and its expression in T or NK cells. FIG. 34A: a combination of CAR, (third generation), sushi/IL-15 is assembled on an expression vector and their expression is driven by the SFFV promoter. CAR with sushi/IL-15 is linked with the P2A cleaving sequence. The sushi/IL-15 portion is composed of IL-2 signal peptide fused to sushi domain and linked to IL-5 via a 26-amino acid poly-proline linker. FIG. 34B: CAR and sushi/IL15 are present on the T or NK cells.

FIGS. 35A-B. CD4IL15RA-CAR expression. FIG. 35A: HEK-293FT cells were transfected with lentiviral plasmids for GFP (lane 1) and CD4IL15RA CAR (lane 2), and positive control, CD4CAR (lane 3). 48 hours after transfection, supernatant was removed, and cells were also removed for a Western blot with mouse anti-human CD3z antibody. FIG. 35B: HEK-293 cells were transduced with either GFP (left) or CD4IL15RA-CAR(right) viral supernatant from transfected HEK-293FT cells. After 3 days incubation, cells were harvested, stained with goat-anti-mouse F(Ab′)2 and analyzed by flow cytometry.

FIG. 36. Transduction of NK cells with CD4IL15RACAR. NK-92 cells were transduced with either GFP (left) or CD4IL15RACAR (right) viral supernatant from transfected HEK-293FT cells. A second transduction was performed 24 hours after the first. 24 hours after the second transduction, cells were harvested, washed and moved to tissue culture plates with fresh media and IL-2. After 3 days incubation, cells were harvested and stained with goat-anti-mouse F(Ab′)2 antibody or goat IgG (control) at 1:250 for 30 minutes. Cells were washed and stained with streptavidin-PE conjugate at 1:500, washed, suspended in 2% formalin, and analyzed by flow cytometry.

FIG. 37. Transduction of T cells with CD4IL15RACAR. Left is the Western blot. HEK-293FT cells were transfected with lentiviral plasmids for GFP (lane 1) and CD4IL15RA-CAR (lane 2). 48 hours after transfection, supernatant was removed, and cells were also collected for a Western blot with mouse anti-human CD3zeta antibody. Right is CD4IL15RACAR expression. Activated T cells from cord blood buffy coat were transduced with either GFP (left) or CD4IL15RACAR (right) viral supernatant from transfected HEK-293FT cells. A second transduction was performed 24 hours after the first. 24 hours after the second transduction, cells were harvested, washed and moved to tissue culture plates with fresh media and IL-2. After 3 days incubation, cells were harvested and stained with goat-anti-mouse F(Ab′)2 or isotype control for 30 minutes. Transduced with either GFP (left) or CD4IL15RA (right). Cells were washed and stained with streptavidin-PE conjugate at 1:250, washed, suspended in 2% formalin, and analyzed by flow cytometry

FIGS. 38A-B. CD4CAR NK-92 cells and CD4IL15RA CAR NK-92 cells eliminate KARPAS 299 T leukemic cells in co-culture. FIG. 38A: NK-92 cells transduced with either GFP control (upper right), CD4CAR (lower left), or CD4IL15RA (lower right) lentiviral supernatant were incubated with KARPAS 299 cells at a ratio of 5:1. After 4 hours co-culture, cells were stained with mouse-anti-human CD4 (APC) and CD3 (PerCp) antibodies and analyzed by flow cytometry (N=2). The upper left panel shows labeled Karpas 299 cells alone. FIG. 38B: The percentage of target cells lysed is shown in the graph.

FIG. 39. CD4CAR NK-92 cells and CD4IL15RA CAR NK-92 cells eliminate MOLT4 T leukemic cells expressing CD4 in co-culture. NK-92 cells transduced with either GFP control (left), CD4CAR (center), or CD4IL15RA (second from right) lentiviral supernatant were incubated with MOLT4 cells at effector:target ratios of 1:1 or 2:1. After overnight co-culture, cells were stained with mouse-anti-human CD4 (APC) and CD56 (PerCp) antibodies and analyzed by flow cytometry (N=2). The upper right panel shows labeled MOLT4 cells alone. The percentage of target cells lysed is shown in the graph.

FIG. 40. CD4IL15RACAR T cells demonstrate more potent anti-leukemic effects in vivo than CD4CAR. NSG mice were sublethally irradiated and intravenously (tail vein) injected the following day with luciferase-expressing MOLM13 cells to induce measurable tumor formation. After 3 days, the mice were intravenously injected with one course of 8 × 10⁶ CD4CAR, or CD4IL15RACAR T cells, or vector control T control cells. On days 3, 6, 9 and 11, mice were injected subcutaneously with RediJect D-Luciferin and subjected to IVIS imaging.

FIG. 41. Percent tumor reduction in mice was measured and compared between the three groups based on the studies from FIG. 40. Average light intensity measured for the CD4CAR and CD4IL15RACAR T injected mice was compared to that of vector control T injected mice, and correlated with remaining tumor burden. In each set of two, CD4CAR T is on the left and CD4IL15RA CAR T is on the right.

FIG. 42. HEK 293 cells were transduced with either EF1-GFP or SFFV-GFP viral supernatant, using the volumes indicated, in DMEM with 10% FBS in a 6 well tissue culture plate. Culture media was changed the following morning. Forty-eight hours later, transduced cells were visualized on an EVOS fluorescent microscope using GFP at 10x.

FIG. 43. HEK 293 cells transduced with either EF1-GFP or SFFV-GFP viral supernatant, using the volumes from the previous figure, were trypsinized, suspended in formalin, and subjected to flow cytometry analysis, using the FITC channel to determine the percentage of GFP+ cells.

FIGS. 44A-B. Activated cord blood buffy coat T cells transduced with either EF1-GFP or SFFV-GFP viral supernatant, with either low or high amounts of viral supernatant, were trypsinized, suspended in formalin, and subjected to flow cytometry analysis, using the FITC channel to determine the percentage of GFP+ cells, 7, 14, 21 and 28 days after transduction.

FIG. 44A: Percent GFP+ T cells for cells transduced with either low or high amounts of supernatant.

FIG. 44B: Percent of GFP+ T cells transduced with the high amount of EF1-GFP supernatant, relative to the percent GFP+ cells in the T cells transduced with the lower amount of SFFV-GFP supernatant. (50 µL of SFFV-GFP and 1 mL of EF1-GFP supernatant was used). (N=2).

FIG. 45. Ligand receptor interactions in malignant plasma cells. The APRIL ligand binds TAC1 or BCMA. The BAFF ligand binds TAC1, BCMA, or BAFF-R.

FIGS. 46A-46D. Production of CD4CAR T cells. FIG. 46A, experimental design. FIG. 46B, CB buffy coat cells were activated 2 days with anti-CD3 antibody and IL-2. Cells were transduced with either GFP (middle) or CD4CAR (right) lentiviral supernatant. After 7 days of incubation, cells were analyzed by flow cytometry with goat anti-mouse Fab2 or goat IgG antibodies conjugated with biotin and followed by streptavidin-PE. Non-transduced, labeled CB cells are shown on the left. FIG. 46C, CD4CAR T cells deplete the CD4+ population during T cell expansion. CB buffy coat cells were activated for 2 days with anti-CD3 antibody and IL-2. CB buffy coat contains two subsets of T cells, CD8+ cytotoxic T cells and CD4+ helper T cells (left). Cells were transduced with either GFP (middle) or CD4CAR (right) lentiviral supernatant. After 3-day culture, cells were analyzed by flow cytometry with mouse-anti-human CD4 (FITC) and CD8 (APC) antibodies. Non-transduced PMBCs were also labeled (left). FIG. 46D, Most CD4CAR T cells have a central memory-like phenotype. CB buffy coat cells were activated 2 days with anti-CD3 antibody. Cells were transduced with CD4CAR lentiviral supernatant. After 6-day expansion, CD8+ cells were analyzed for CD62L, CD45RO and CD45RA phenotypes by flow cytometry (N=3).

FIGS. 47A-47D. CD4CAR T cells eliminate T-cell leukemic cells in co-culture assays. FIG. 47A, CD4CAR T cells eliminate KARPAS 299 T-cell leukemic cells in co-culture. Activated human CB buffy coat cells transduced with either GFP (middle) or CD4CAR (right) lentiviral supernatant were incubated with KARPAS 299 cells at a ratio of 2:1. After 24 hours co-culture, cells were stained with mouse anti-human CD4 (APC) and CD8 (PerCp) antibodies and analyzed by flow cytometry for T cell subsets (N=3). FIG. 47B and FIG. 47C, CD4CAR T cells eliminate primary T- cell leukemic cells in co-culture. Activated human CB buffy coat cells transduced with either GFP (middle) or CD4CAR (right) lentiviral supernatant were incubated with primary T-cell leukemia cells from Sezary syndrome and PTCLs (FIG. 47C) at a ratio of 2:1. After 24 hours of co-culture, cells were analyzed by flow cytometry with mouse-anti-human CD4 (FITC) and CD8 (APC) antibodies (N=3). Human primary cells alone are also labeled (left). FIG. 47D: CD4CAR T cells were unable to lyse CD4-negative lymphoma cells (SP53, a B-cell lymphoma cell line). Activated human CB buffy coat cells transduced with either GFP (middle) or CD4CAR (right) lentiviral supernatant were incubated with SP53 mantle cell lymphoma cells which were pre-stained with the membrane dye CMTMR, at a ratio of 2:1. After 24 hours co-culture, cells were stained with mouse anti-human CD3 (PerCp) and then analyzed by flow cytometry (N=2). SP53 cells alone, pre-stained with CMTMR were also labeled (left).

FIGS. 48A-48B. CD4CAR T cells derived from PBMCs are highly enriched for CD8+ T and specifically kill CD4-expressing leukemic cell lines. FIG. 48A: CD4CAR T cells derived from PBMCs are highly enriched for CD8+ T cells. PMBC buffy coat cells constituting T cells, CD8+ and CD4+ (left) were activated for 2 days with anti-CD3 antibody and IL-2, then transduced with either GFP (middle) or CD4CAR (right) lentiviral supernatant. After 3 days of culture, cells were labeled and analyzed by flow cytometry for T cell subsets. Non-transduced PMBCs were also labeled (left). FIG. 48B: CD4CAR T cells specifically kill KARPAS 299 cells. PMBC T cells transduced with either GFP control or CD4CAR lentiviral supernatant were incubated with CFSE-stained KARPAS 299 at the ratios of 2:1, 5:1 and 10:1, respectively. After overnight incubation at 37° C., dye 7AAD was added, and the cells were analyzed by flow cytometry. Percent killing of target cells is measured by comparing survival of target cells relative to the survival of negative control cells (SP53 cells, a B-cell lymphoma cell line stained with CMTMR).

FIGS. 49A-49D. CD4CAR T cells efficiently mediate anti-leukemic effects in vivo with different modes. NSG mice received 2.5 Gy for sub-lethal irradiation. Twenty-four hours after irradiation, mice were injected subcutaneously with either 1×10⁶ (in 4A) or 0.5×10⁶ (in 4B and 4C) KARPAS 299 cells. Injected mice were treated with different courses and schedules of CD4CAR T cells or control T cells. N=5 for each group of injected mice. FIG. 49A, a low dose of 2×10⁶ of CD4CAR T cells was injected on day 3 followed by a large dose, 8×10⁶, of CD4CAR T cells on day 22 after upon observed acceleration of tumor growth. FIG. 49B, two large doses of CD4CAR T cells, 8×10⁶ and 5.5×10⁶ were injected on day 3 and 10 respectively. FIG. 49C, a repeat low dose (2.5×10⁶) of CD4CAR T cells was injected every 5 days for a total of four administrations. FIG. 49D, overall survival of mice treated with the indicated CD4CAR T cells or control GFP T cells. N=10.

FIG. 50. Co-culture specificity and dose response killing curve. CD4CAR NK cells lyse CD4-expressing leukemic cell lines in a dose dependent and specific manner. CD4CAR NK and vector control cells were incubated with an equal ratio of CFSE-stained “on-target” (Karpas 299 or CCRF-CEM) cells and CMTMR-stained “off target” MOLT4 cells at 1:4, 1:2, and 1:1 effector to target ratios. After 24 hours, 7-AAD dye was added and remaining live cells were analyzed by flow cytometry. Percent killing of target cells was measured by comparing CD4⁺ Karpas 299 or CCRF-CEM cell survival in CD4CAR NK cell co-cultures relative to that in vector control NK cell co-cultures.

FIGS. 51A-51B. CD4CAR NK cells eliminate CD4⁺ T-cells isolated from human cord blood at an effector to target ratio of 2:1, but do not affect hematopoietic stem cell/progenitor compartment output. FIG. 51A: Co-culture assays were performed at an effector to target ratio of 2:1 for 24 hours, after which, cells were stained with mouse anti-human CD56 and CD4 antibodies. Target cells were incubated alone as a control (left). NK cells were transduced with either vector control (center) or CD4CAR (right) lentiviral supernatant and incubated with CD4⁺ T-cells obtained from human cord blood. (N=2). FIG. 51B: CD4CAR NK cells were incubated at co-culture effector:target ratios of 2:1 and 5:1 respectively with 500 CD34+ cord blood cells for 24 hours in NK cell media supplemented with IL-2. Experimental controls used were CD34+ cells alone, and non-transduced NK cells were co-cultured at respective 2:1 and 5:1 effector:target ratios with CD34+ CB cells. Hematopoietic compartment output was assessed via formation of erythroid burst-forming units (BFU-E) and number of granulocyte/monocyte colony-forming units (CFU-GM) at Day 16. CFU statistical analysis was performed via 2-way ANOVA with alpha set at 0.05.

FIGS. 52A-52D. CD4CAR NK cells demonstrate anti-leukemic effects in vivo. NSG mice were sublethally irradiated and intradermally injected with luciferase-expressing Karpas 299 cells (Day 0) to induce measurable tumor formation. On day 1 and every 5 days for a total of 6 courses, mice were intravenously injected with 5 × 10⁶ CD4CAR NK cells or vector control NK control cells. FIG. 52A: On days 7, 14, and 21, mice were injected subcutaneously with RediJect D-Luciferin and subjected to IVIS imaging. FIG. 52B: Average light intensity measured for the CD4CAR NK injected mice was compared to that of vector control NK injected mice. FIG. 52C: On day 1, and every other day after, tumor size area was measured and the average tumor size between the two groups was compared. FIG. 52D: Percent survival of mice was measured and compared between the two groups.

FIGS. 53A-53C. Generation of CD5CAR. The DNA gene construct and the translated protein construct for CD5CAR, and anchored CD5 scFv antibody and a cartoon demonstrating the creation and function of CD5CAR. The DNA construct of the third generation CD5CAR construct from 5′ to 3′ reads: Leader sequence, the anti-CD5 extracellular single chain variable fragment (Anti-CD5 ScFv), the hinge region, the trans-membrane region, and the three intracellular signaling domains that define this construct as a 3rd generation car; CD28, 4-1BB and CD3ζ. The DNA construct of the anchored CD5 scFv antibody is the same as the CD5CAR construct without the intracellular signaling domains, as is the translated protein product for anchored CD5 scFv antibody. The translated protein constructs contain the anti-CD5 ScFv that bind to the CD5 target, the hinge region that allows for appropriate positioning of the anti-CD5 ScFv to allow for optimal binding position, and the trans-membrane region. The complete CD5CAR protein also contains the two co-stimulatory domains and an intracellular domain of CD3 zeta chain. This construct is considered as a 3rd generation CAR: CD28, 4-1BB, and CD3ζ (FIG. 53A). Western blot analysis demonstrates the CD5CAR expression in HEK293 cells. HEK293 cells which had been transduced with GFP (as negative control) or CD5CAR lentiviruses for 48 h were used for Western blot analysis using CD3ζ antibody to determine the expression of CD5CAR. Left lane, the GFP control HEK293 cells, with no band as expected. The right lane showing a band at about 50 kDa, the molecular weight that we expected based on the CD5CAR construct (FIG. 53B). Flow cytometry analysis for CD5CAR expression on T cells surface for lentiviral transduced CD5CAR T cells. This analysis was performed on the double transduced CD5CAR T cells at day 8 after the second lentiviral transduction. Isotype control T cell population (negative control) vs. transduced T cells expressing CD5 CAR showing 20.53% on T cells by flow cytometry using goat anti-mouse F(AB’)2-PE (FIG. 53C).

FIG. 54. Comparisons of single and double transductions with CD5 CAR lentiviruses in the downregulation of surface CD5 expression on the T cells. The downregulation of extracellular CD5 protein versus GFP T-cell control over 8 days following lentiviral transduction is analyzed. The single transduced CD5CAR T-cells do not show complete downregulation of CD5 from cell surface by day 8, with a maximum decrease in CD5 protein expression on day 6. In the double transduced population, we note the decrease in the absolute number of CD5+, CD3+ double positive CD5CAR T-cells over time, from 24.44% on day 0 to a near complete reduction of CD5 expression on day 4. In contrast, the GFP T-cell control maintains a CD5+, CD3+ double positive population above 95% from day 2 through day 8.

FIGS. 55A-55B. CD5CAR cells effectively lyse T -ALL cell lines that express CD5, and do not lyse a T leukemic cell line that does not express CD5. FIG. 55A: Flow cytometry analysis of T-ALL cell lines alone (left column), in co-culture with GFP vector transduced T-cells (middle row) and in co-culture with CD5CAR transduced T-cells (right row). Each cell line is seen in each row, The CD5+ T-ALL cell lines in the top and middle rows (CCRF-CEM and Molt-4) with the CD5 negative cell line seen as the bottom row (KARPAS 299). KARPAS 299 is a CD5 negative T cell lymphoma. The incubation time for all co-cultures was 24 hrs, with an effector:target cell ratio of 5:1. The cell lysis compared to GFP control was over 78% for both CD5 T ALL leukemic cell lines, compared to that for the GFP control. FIG. 55B: This bar graph denotes the T cell lysis achieved by the CD5CAR T-cells when compared to the GFP T-cells co-culture described in FIG. 55A. There was no lysis observed in CD5 CAR T cells co-cultures with KARPAS 299, which is CD5 negative (n=3 independent experiments done in duplicate).

FIGS. 56A-56D. CD5CAR cells effectively lyse T-cell acute lymphoblastic leukemic cells from patient samples that express CD5. FIG. 56A: Flow cytometry analysis of T-ALL cells alone (left column), in co-culture with GFP T-cells (middle row) and in co-culture with CD5CAR T-cells (right row). Each patient cells are given a row and are numbered to maintain patient confidentiality. The incubation time for all co-cultures was 24 hrs, with an effector:target cell ratio of 5:1. The cell lysis compared to GFP control was over 71.3% for the T-ALL -1 compared to control. The rest of the cell lines demonstrated positive cell lysis as well, but to a lesser degree, between 33-47%. This may be related to the CD5 expression for each leukemic sample, which is discussed below. FIG. 56B: This bar graph denotes the T cell lysis achieved by the CD5CAR T-cells when compared to the GFP T-cell co-culture described in FIG. 21A. FIG. 56C: Flow cytometry analysis of the levels of CD5 expression on a panel of four patient sample T-ALL cell populations. FIG. 56D: The difference of mean fluorescent intensity (MFI) of CD5 expression among human samples was determined by flow cytometry analysis.

FIG. 57. Analysis of CD5CAR T-cell killing ability for patient T-ALL cells (T-ALL-8) in details. Flow cytometry analysis demonstrating CD5CAR T-cell killing ability for patient’s T-ALL cells. The control GFP-T cell and T-ALL-8 cell co-culture are seen on the left, and the CD5CAR co-culture with T-ALL 8 is seen on the right. We note avid lysis of all CD5 positive cells, both CD34 positive (circled in red) and CD34 negative (circled in green, T cells), with no lysis noted for CD5 negative cells. When compared to GFP control, CD5CAR T cells lyse at minimum 93.1% of CD5 positive T-ALL-8 cells when compared to GFP control. Experiment was done in duplicate. In addition, CD5CAR T cells essentially eliminate the T cell population (CD5+CD34-, circled in green).

FIGS. 58A-58B. CD5CAR T cells effectively eliminate normal GFP labeled T cells. FIG. 58A: CD5CAR T cells kill normal T cells in a dose dependent manner. CD5CAR T cells or CD123CAR T cells (control) were co-cultured with GFP labeled T cells at 0.25:1, 0.5:1 and 1:1 effector to target ratios. After 24 hours, remaining live GFP T cells were analyzed by flow cytometry. Percent killing of target cells was measured by comparing GFP T cell survival in CD5 co-cultures relative to that in control CD123CAR T cells as T cells do not express CD123. FIG. 58B: Co-culture killing curve based on the data from 13A.

FIGS. 59A-59C. Co-culture assays were performed to determine if normal T cells maintained CD5 expression when they were co-cultured with CD5CAR or anchored CD5 scFv T cells or CD123CAR (control) for 2 days (FIGS. 59A and 59B) or 4 days (FIG. 59C) at a ratio of 1:1. CCRF-CEM or Molt-4 T ALL cells were transduced with lentiviruses expressing CD5CAR or anchored CD5 scFv. After the second transduction, the transduced leukemic cells were analyzed for CD5 expression by flow cytometry.

FIG. 60. CD5CAR T cells demonstrate profound anti-leukemic effects in vivo. NSG mice were sublethally irradiated and, after 24 hours, intravenously injected with 1 × 10⁶ luciferase-expressing CCRF-CEM cells (Day 0) to induce measurable tumor formation. On day 3 and 4, mice were intravenously injected with 5 × 10⁶ CD5CAR T cells or vector control T cells. These injections were repeated on Days 6 and 7, for a total of 2.0 × 10⁷ cells per mouse. On days 5, 8, 10 and 13, mice were injected subcutaneously with RediJect D-Luciferin and subjected to IVIS imaging.

FIGS. 61A-61B. The CD5CAR NK cells (NK-92) effectively eliminate CCRF-CEM T-ALL cell line in vitro. FIG. 61A and FIG. 61B, T-lymphoblast cell line CCRF-CEM expressing CD5 was co-cultured with CD5 CAR NK cells in the indicated E:T (effector:target) cell ratios for 24 hours. Target populations were quantified with flow cytometry using CD56 and CD5 to separate the NK-CAR and target cell population respectively. Cell survival is expressed relative to transduced vector control NK cells and each bar graph represents the average statistics for duplicate samples with N=2.

FIGS. 62A-62B. CD5CAR NK cells demonstrate potent anti-leukemic effects in vivo. NSG mice were sublethally irradiated and, after 24 hours, intravenously injected with 1 × 10⁶ luciferase-expressing CCRF-CEM cells (Day 0) to induce measurable tumor formation. On day 3 and 4, mice were intravenously injected with 5 × 10⁶ CD5CAR NK cells or vector control NK cells. These injections were repeated on Days 6 and 7, for a total of 2.0 × 10⁷ cells per mouse. FIG. 62A, on day 5, mice were injected subcutaneously with RediJect D-Luciferin and subjected to IVIS imaging. FIG. 62B, Percentage of tumor cells killed in mice treated with CD5CAR NK cells relative to control.

FIGS. 63A-63B. Organization of CD3CAR and its expression. FIG. 63A: Schematic representation of the organization of CD3CAR in lentiviral vectors. CAR expression is driven by a SFFV (spleen focus-forming virus) promoter and as a 3^{rd} generation construct, contains a leader sequence, the anti-CD3scFv, a hinge domain (H), a transmembrane domain (TM), two co-stimulatory domains of CD28 and 4-BB and the intracellular signaling domain of CD3 zeta. FIG. 63B: HEK-293FT cells were transduced with lentiviral plasmids for GFP (lane 1) and CD3CAR (lane 2) for Western blot analysis at 48 h post transduction and probed with mouse anti-human CD3zeta antibody.

FIGS. 64A-64B. CD3CAR NK cells eliminate CD3-expressing T-ALL cell lines in vitro. FIG. 64A, T-lymphoblast cell line Jurkat expressing approximately 80% CD3 was co-cultured with CD3CAR NK cells in the indicated E:T (effector:target) cell ratios for 6 hours. FIG. 64B: Sorted (CCRF-CD3) or unsorted CCRF-CEM (CCRF-CEM) cells were co-cultured with CD3CAR NK cells for 24 hours. Target populations were quantified with flow cytometry using CD56 and CD3 to separate the NK-CAR and target cell population respectively. Cell survival is expressed relative to transduced vector control NK cells and each bar graph represents the average statistics for duplicate samples with N=2 experiments. The CD3CAR NK cells display robust killing ability for primary CD3+ leukemic cells from patient samples.

FIG. 65. C0D3CAR NK cells demonstrate profound anti-leukemic effects in vivo. NSG mice were sublethally irradiated and, after 24 hours, intravenously injected with 1 × 10⁶ luciferase-expressing Jurkat cells (Day 0) to induce measurable tumor formation. On day 3 and 4 mice were intravenously injected with 5 × 10⁶ CD3CAR NK cells or vector control NK cells each day. These injections were repeated on Days 6 and 7, and again on Day 10, for a total of 2.5 × 10⁷ cells per mouse. On days 4, 7, 9, and 13, mice were injected subcutaneously with RediJect D-Luciferin and subjected to IVIS imaging.

FIG. 66. Three pairs of sgRNA per gene are designed with CHOPCHOP to target CD2, CD3, CD5 and CD7. Three pairs of sgRNA were designed with CHOPCHOP to target the gene of interest. Gene-specific sgRNAs were then cloned into the lentiviral vector (Lenti U6-sgRNA-SFFV-Cas9-puro-wpre) expressing a human Cas9 and puromycin resistance genes linked with an E2A self-cleaving linker. The U6-sgRNA cassette is in front of the Cas9 element. The expression of sgRNA and Cas9puro is driven by the U6 promoter and SFFV promoter, respectively.

FIGS. 67A-67D. Generation of stable CD5-deficient CCRF-CEM and MOLT-4 T cells using CRISPR/Cas9 lentivirus system. FIG. 67A: Flow cytometry analysis demonstrating the loss of CD5 expression in CCRF-CEM T-cells with CRISPR/Cas9 KD using two different sgRNAs, Lenti-U6-sgCD5a-SFFV-Cas9puro (sgCD5A) and Lenti-U6-sgCD5b-SFFV-Cas9puro (sgCD5B) after puromycin selection. Wild type control is seen in the left most scatter plot. Because the CRISPR/Cas9 KD technique with sgRNA CD5A was more successful at CD5 protein downregulation, this population (denoted by the blue circle and arrow) was selected for sorting, purification and analysis in FIG. 22B. FIG. 67B: Flow cytometry analysis data indicating the percentage of purely sorted stable CD5 negative CCRF-CEM cells transduced using the scCD5A CRISPR/Cas9 technique. We note the >99% purity of CD45 positive, CD5 negative CCRF sgCD5A T-cells. FIG. 67C: Flow cytometry analysis demonstrating the loss of CD5 expression in MOLT-4 T-cells with CRISPR/Cas9 KD using two different sgRNA sequences (sequence CD5A and CD5B, middle and right columns) after puromycin treatment. Wild type control is seen the leftmost scatter plot. Because the CRISPR/Cas9 KD technique with primer CD5A was more successful at CD5 protein downregulation, this population (denoted by the blue circle and arrow) was selected for sorting, purification and analysis in FIG. 22D. FIG. 67D: Flow cytometry analysis data indicating the percentage of purely sorted stable CD5 negative MOLT-4 cells transduced using the scCD5A CRISPR/Cas9 technique. We note the >99% purity of CD45 positive, CD5 negative MOLT-4 sgCD5A T-cells.

FIGS. 68A-68D. Generation and cell sorting of stable CD7 loss in CCRF-CEM cells or NK-92 cells using CRISPR/Cas9 lentivirus system. The percentage of CD7 loss in CCRF-CEM (FIGS. 68A and 68B) or NK-92(FIGS. 68C and 68D) using sgCD7A (Lenti-U6-sgCD7a-SFFV-Cas9-puro) and sgCD7B (Lenti-U6-sgCD7b-SFFV-Cas9-puro) was determined by flow cytometric analysis with CD45 and CD7 antibodies after puromycin treatment. The values of insert in figures showed percentage of positive and negative expressing CD45 or CD7 among analysis. Right panel indicated the percentage purity of sorted stable CD7 negative cells in CCRF-CEM (FIG. 68B) or in NK-92 cells (FIG. 68D) prepared from CD7 negative cells transduced using sgCD7A or sgCD7D CRISPR lentiviruses.

FIGS. 69A-69C. CD2CAR NK cells eliminate T-cell leukemic cells in co-culture assays. FIG. 69A: CD2CAR NK cells eliminate leukemic cells from T-ALL patient’s cells in co-culture. NK-92 cells transduced with either GFP (top) or CD2CAR (bottom) lentiviral supernatant were incubated with primary human T-ALL cells, SAMPL1 (PT1) at a ratio of 5:1 (1 for 100,000 cells). After 24 hours co-culture, cells were stained with mouse anti-human CD2 (APC) antibodies and analyzed by flow cytometry (N=2). FIG. 69B: CD2CAR NK cells eliminate a T-ALL cell line, CCRF leukemic cells in co-culture. NK-92 cells transduced with either GFP (top) or CD2CAR (bottom) lentiviral supernatant were incubated with CCRF cells at a ratio of 5:1 (1 for 100,000 cells). CCRF cells were pre-stained with cell tracker dye (CMTMR). After 24 hours co-culture, cells were stained with mouse anti-human CD2 (APC) antibodies and analyzed by flow cytometry (N=2). FIG. 69C: Percentage of target cells (CCRF or PT1) lysed compared to GFP NK experimental control. At a 5:1 ratio and 24 hours co-culture, CD2CAR NK cells were able to eliminate about 60% of CD2 positive leukemic cells in co-culture assays.

FIGS. 70A-70B. CD7CAR NK⁷⁻-92 cells effectively lyse T cell ALL cell line T cells that express CD7.To avoid self-killing, CD7 deficient NK-92 (NK⁷⁻-92) cells were generated and transduced with CD7CAR. Two transduced preparations of CD7CAR NK⁷⁻-92 cells, #A and #B were used to test their killing ability. FIG. 70A: Flow cytometry analysis of CCRF-CEM cells alone (left column), in co-culture with GFP NK⁷⁻-92 cells (middle column), and in co-culture with CD7CAR-NK-92-cells, #A and B# (right columns). FIG. 70B: bar graphs based on data obtained from (FIG. 70A).

FIG. 71. Schematic representation of recombinant lentiviral vectors encoding CD7CAR (also called CD7 RTX CAR). CD7CAR consists of a humanized anti-CD7 scFv, CD8 hinge and transmembrane regions, and a CD28 co-activator fused to the CD3zeta signaling domain. The hinge region of CD7CAR also contains two RTX-binding epitopes. Expression is driven by the spleen focus-forming virus (SFFV) promoter.

FIGS. 72A-72D: FIG. 72A: Characterization of CD7CAR. Staining with goat-anti-mouse F(Ab′)2-Pe revealed a CAR expression of approximately ~70%. FIG. 72B: Staining with anti-human CD34 (used to detect the RTX-binding epitope) also demonstrated a transduction efficiency of ~80%. FIG. 72C: Staining with anti-human CD3 and anti-human CD7 demonstrate that the CD7CAR T-cells retain CD3 expression but lose expression of CD7. FIG. 72D: CD7CAR T-cells are able to expand at rates similar to control T-cells despite losing CD7 expression.

FIG. 73: CD7CAR demonstrates potent cytotoxicity against CD7+ cell lines in vitro. CEM-CCRF cells are ~90% CD7+ (bottom right panel). Control T-cells (left panels) or CD7-RTX T-cells (right panels) were placed with CEM-CCRF cells in an 18-h co-culture at E:T ratios of 1:1 (first row), or 2:1 (second row). Target CD7+ cells are circled in each panel. The results of the co-culture experiments demonstrate that CD7CAR T-cells lyse 99.88% and 99.82% of the CEM-CCRF cells relative to control at 1:1 and 2:1 respectively.

FIGS. 74A-74D: CD7CAR improves outcome in in vivo model of T-ALL. FIG. 74A: NSG mice were sub-lethally irradiated and then intravenously injected on Day 1 with 1.0×10⁶ luciferase-expressing CEM-CCRF cells. Five days later, mice were injected with 10×10⁶ control or CD7CAR T-cells. Mice were injected with RediJect D-Luciferin on Days 5, 10, 13, 16, and 19 and subjected to IVIS imaging. Dorsal view. FIG. 74B: Total flux (photons/second) was measured and indicated a statistically significant difference in tumor burden between the two groups as early as day 8. FIG. 75C: The flux of the CD7CAR-treated mice had 41.3% (dorsal) less tumor by day 8, which increased to 99.6% (dorsal) by day 19. FIG. 75D: While all the control mice required euthanasia due to hindlimb paralysis and hunchback by day 24-26, the CD7CAR-treated mice survived significantly longer, remaining alive on Day 43. Kaplan-Meier survival analysis curve (p = 0.0026).

FIG. 75: depicted is prior to CAR T cell therapy, the patient leukemic blast distribution; peripheral blood by flow cytometry yields 0.39%, bone marrow morphology is 24% and bone marrow by flow cytometry is 6.13%. The patient received the standard preconditioning therapy, fludarabine and cytarabine. Four days post preconditioning treatment the first dose of CD7 CAR T cell treatment was administered at 1×10^6/kg and in the subsequent two days 2.0×10^6/kg dose was administered. The patient achieved complete remission. MRD (minimal residual disease) negative in the peripheral blood and bone marrow for leukemic blasts.

FIG. 76: shows 6 days (left panel) prior to CAR T cell therapy, T cell populations were positive for CD7 surface protein. Following CAR T cell treatment, the T cell populations comprised of almost all CD3+ CD7-negative cells (right three panels).

FIG. 77: the summary indicates that the absolute CBC (complete blood count), lymphocyte subtype, chimerism, percentage CAR T expression in BM and PB. See diagram / boxes. Bottom panel the hexagonal shape indicates absence of leukemic cells (right and middle panels). Bottom right, bone marrow flow cytometry analysis indicating near complete absence of CD7 surface protein in all T cells. WBC: white blood cell; NEU: neutrophils; LYM: lymphocyte; HGB: Hemoglobin; PLT: platelet; CIK: cytokine-induced killer cells; PB: peripheral blood; BM: bone marrow; GVHD: Graft-versus-host disease.

FIG. 78. Construction of anti-TAA-anit-CD3-ENG in order to expression of anti-TAA-anti-CD3 engager. Viral construct contains the cDNA encoding an immunoglobulin heavy-chain leader peptide preceding an anti-TAA scFv and anti-CD3 scFv with a short serine-glycine linker and myc-tag in tandem. TAA is called tumor associated antigen. G4S linker: GGGGS

FIG. 79. Construction of CD7 CAR and a CAR, X that requires CAR T expansion and reduction of toxicity. The CD7CAR can be combined with a CAR, X to select from the following target antigens: at least one of this group, but not limited to, GD2, GD3,, ROR1, PSMA, PSCA (prostate stem cell antigen), MAGE A3, Glycolipid, glypican 3, F77, GD-2, WT1, CEA, HER-2/neu, MAGE-3, MAGE-4, MAGE-5, MAGE- 6, alpha-fetoprotein, CA 19-9, CA 72-4, NY-ESO, FAP, ErbB, c-Met, MART-1, MUC1, MUC2, MUC3, MUC4, MUC5, KIF20A, Survivin, AFP-1, gp100, MUC1, PAP-10, PAP-5, TRP2-1, SART-1, VEGFR1, VEGFR2, NEIL3, MPHOSPH1, DEPDC1, FOXM1, CDH3, TTK, TOMM34, URLC10, KOC1, UBE2T, TOPK, ECT2, MESOTHELIN, NKG2D, P1A, GM2, CD30, MMG49 epitope, EGFRvIII, CD33, CD123, CLL-1 (CD371), immunoglobin kappa and lambda, CD38, CD52, CD47, CD200, CD70, CD19, CD20, CD22, CD38, BCMA, CS1, NKG2D receptor, April receptor, BAFF receptor, TACI, CD3, CD4, CD8, CD5, CD2, GPRC5D (G protein-coupled receptor, class C, group 5, member D) and CD138. The target antigens can also include viral or fungal antigens, such as E6 and E7 from the human papillomavirus (HPV) or EBV (Epstein Barr virus) antigens. A schematic representation of CD7 compound CAR (cCAR). The construct comprises a promoter driving the expression of two units of CARs linked by a cleaving site such as P1A, E2A, T2A and F2A. Upon cleavage of the linker peptide, the cCARs split and engage upon targets expressing CD7 and any another antigen. As a novel CD7 cCAR construct, the co-stimulatory domains of the construct may include, but is not limited to, 4-1BB or CD28. Co-stimulatory domain in each CAR can be the same or different. CD7CAR consists of an anti-CD7 scFv, CD8 hinge (H) and transmembrane (TM) regions, and a co-activator fused to the CD3zeta signaling domain. The hinge region of CD7CAR also contains two CD20 RTX-binding epitopes. Expression is driven by a single promoter.

FIG. 80. A schematic representation of cCAR construct (CD7-CD19 cCAR). The construct comprises a single promoter driving the expression of two modular units of CARs linked by a P2A peptide. Upon cleavage of the linker, the cCARs split and engage upon targets expressing CD7 and/or CD19. As a novel cCAR construct, the co-stimulatory domains of the construct may include, but is not limited to, 4-1BB on the CD7 CAR segment and a CD28 region on the CD19 CAR. Co-stimulatory domain in each CAR can be the same or different. Each CAR consists of a scFv, CD8 hinge (H) and transmembrane (TM) regions, and a co-activator fused to the CD3zeta signaling domain. The hinge region of CD7CAR also contains two CD20 RTX-binding epitopes. The CD7-CD19 cCAR is designed to target autoreactive T cells expressing CD7 and autoreactive B cells expressing CD19.

FIG. 81. Expression of BCMA-CD19-VAC cCAR T-cells. Staining with goat-anti-mouse F(Ab′)2-Pe revealed a CAR expression of approximately ~28.18% by flow cytometry analysis. The viruses were obtained from a BCMA-CD19 CAR stable producer RD114 cell line.

FIG. 82. Expression of CLL1-CD33 VAC cCAR (also called CLL1-CD33b VAC)T-cells. Flow cytometry analysis showed that ~31.32% of T-cells expressed the CLL-1-CD33 cCAR by staining with goat-anti-mouse F(Ab′)2-Pe. The viruses were obtained from a single clone derived from BCMA-CD19 CAR stable producer RD114 cell line.

### DETAILED DESCRIPTION

The disclosure provides chimeric antigen receptor (CAR) compositions, methods of making and using thereof.

A chimeric antigen receptor (CAR) polypeptide includes a signal peptide, an antigen recognition domain, a hinge region, a transmembrane domain, at least one co-stimulatory domain, and a signaling domain.

First-generation CARs include CD3z as an intracellular signaling domain, whereas second-generation CARs include at least one single co-stimulatory domain derived from various proteins. Examples of co-stimulatory domains include, but are not limited to, CD28, CD2, 4-1BB (CD137, also referred to as “4-BB”), and OX-40 (CD124). Third generation CARs include two co-stimulatory domains, such as, without limiting, CD28, 4-1BB, CD134 (OX-40), CD2 and/or CD137 (4-1BB).

As used herein, the terms “peptide,” “polypeptide,” and “protein” are used interchangeably, and refer to a compound having amino acid residues covalently linked by peptide bonds. A protein or peptide must contain at least two amino acids, and no limitation is placed on the maximum number of amino acids that can include a protein’s or peptide’s sequence. Polypeptides include any peptide or protein having two or more amino acids joined to each other by peptide bonds. As used herein, the term refers to both short chains, which also commonly are referred to in the art as peptides, oligopeptides, and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

A “signal peptide” includes a peptide sequence that directs the transport and localization of the peptide and any attached polypeptide within a cell, e.g. to a certain cell organelle (such as the endoplasmic reticulum) and/or the cell surface.

The signal peptide is a peptide of any secreted or transmembrane protein that directs the transport of the polypeptide of the disclosure to the cell membrane and cell surface, and provides correct localization of the polypeptide of the present disclosure. In particular, the signal peptide of the present disclosure directs the polypeptide of the present disclosure to the cellular membrane, wherein the extracellular portion of the polypeptide is displayed on the cell surface, the transmembrane portion spans the plasma membrane, and the active domain is in the cytoplasmic portion, or interior of the cell.

In one embodiment, the signal peptide is cleaved after passage through the endoplasmic reticulum (ER), i.e. is a cleavable signal peptide. In an embodiment, the signal peptide is human protein of type I, II, III, or IV. In an embodiment, the signal peptide includes an immunoglobulin heavy chain signal peptide.

The “antigen recognition domain” includes a polypeptide that is selective for or targets an antigen, receptor, peptide ligand, or protein ligand of the target; or a polypeptide of the target.

The antigen recognition domain may be obtained from any of the wide variety of extracellular domains or secreted proteins associated with ligand binding and/or signal transduction. The antigen recognition domain may include a portion of Ig heavy chain linked with a portion of Ig light chain, constituting a single chain fragment variable (scFv) that binds specifically to a target antigen. The antibody may be monoclonal or polyclonal antibody or may be of any type that binds specifically to the target antigen. In another embodiment, the antigen recognition domain can be a receptor or ligand. In particular embodiments, the target antigen is specific for a specific disease condition and the disease condition may be of any kind as long as it has a cell surface antigen, which may be recognized by at least one of the chimeric receptor construct present in the compound CAR architecture. In a specific embodiment, the chimeric receptor may be for any cancer for which a specific monoclonal or polyclonal antibody exists or is capable of being generated. In particular, cancers such as neuroblastoma, small cell lung cancer, melanoma, ovarian cancer, renal cell carcinoma, colon cancer, Hodgkin’s lymphoma, and childhood acute lymphoblastic leukemia have antigens specific for the chimeric receptors.

The target specific antigen recognition domain preferably includes an antigen binding domain derived from an antibody against an antigen of the target, or a peptide binding an antigen of the target, or a peptide or protein binding an antibody that binds an antigen of the target, or a peptide or protein ligand (including but not limited to a growth factor, a cytokine, or a hormone) binding a receptor on the target, or a domain derived from a receptor (including but not limited to a growth factor receptor, a cytokine receptor or a hormone receptor) binding a peptide or protein ligand on the target.

In one embodiment, the antigen recognition domain includes the binding portion or variable region of a monoclonal or polyclonal antibody directed against (selective for) the target.

In another embodiment, the antigen recognition domain includes Camelid single domain antibody, or portions thereof. In one embodiment, Camelid single-domain antibodies include heavy-chain antibodies found in camelids, or VHH antibody. A VHH antibody of camelid (for example camel, dromedary, llama, and alpaca) refers to a variable fragment of a camelid single-chain antibody (See Nguyen et al, 2001; Muyldermans, 2001), and also includes an isolated VHH antibody of camelid, a recombinant VHH antibody of camelid, or a synthetic VHH antibody of camelid.

In another embodiment, the antigen recognition domain includes ligands that engage their cognate receptor. By way of example, APRIL is a ligand that binds the TAC1 receptor or the BCMA receptor. In accordance with an invention disclosed herein, the antigen recognition domain includes APRIL, or a fragment thereof. By way of further example, BAFF is a ligand that binds the BAFF-R receptor or the BCMA receptor. In accordance with an invention disclosed herein, the antigen recognition domain includes BAFF, or a fragment thereof. In another embodiment, the antigen recognition domain is humanized.

It is understood that the antigen recognition domain may include some variability within its sequence and still be selective for the targets disclosed herein. Therefore, it is contemplated that the polypeptide of the antigen recognition domain may be at least 95%, at least 90%, at least 80%, or at least 70% identical to the antigen recognition domain polypeptide disclosed herein and still be selective for the targets described herein and be within the scope of the disclosure.

The target includes interleukin 6 receptor, NY-ESO-1, alpha fetoprotein (AFP), glypican-3 (GPC3), BCMA, BAFF-R, TACI, LeY, CD5, CD13, CD14, CD15 CD19, CD20, CD22, CD33, CD41, CD61, CD64, CD68, CD117, CD123, CD138, CD267, CD269, CD38, Flt3 receptor, CS1, CD45, ROR1, PSMA, MAGE A3, Glycolipid, glypican 3, F77, GD-2, WT1, CEA, HER-2/neu, MAGE-3, MAGE-4, MAGE-5, MAGE- 6, alpha-fetoprotein, CA 19-9, CA 72-4, NY-ESO, FAP, ErbB, c-Met, MART-1, CD30, EGFRvIII, immunoglobin kappa and lambda, CD38, CD52, CD3, CD4, CD8, CD5, CD7, CD2, and CD138

In another embodiment, the target includes any portion interleukin 6 receptor, NY-ESO-1, alpha fetoprotein (AFP), glypican-3 (GPC3), BCMA, BAFF-R, TACI, LeY, CD5, CD13, CD14, CD15 CD19, CD20, CD22, CD33, CD41, CD61, CD64, CD68, CD117, CD123, CD138, CD267, CD269, CD38, Flt3 receptor, CS1, CD45, TACI, ROR1, PSMA, MAGE A3, Glycolipid, glypican 3, F77, GD-2, WT1, CEA, HER-2/neu, MAGE-3, MAGE-4, MAGE-5, MAGE- 6, alpha-fetoprotein, CA 19-9, CA 72-4, NY-ESO, FAP, ErbB, c-Met, MART-1, CD30, EGFRvIII, immunoglobin kappa and lambda, CD38, CD52, CD3, CD4, CD8, CD5, CD7, CD2, and CD138.

In one embodiment, the target includes surface exposed portions of interleukin 6 receptor, NY-ESO-1, alpha fetoprotein (AFP), glypican-3 (GPC3), BCMA, BAFF-R, TACI, LeY, CD5, CD13, CD14, CD15 CD19, CD20, CD22, CD33, CD41, CD61, CD64, CD68, CD117, CD123, CD138, CD267, CD269, CD38, Flt3 receptor, CS1, CD45, TACI, ROR1, PSMA, MAGE A3, Glycolipid, glypican 3, F77, GD-2, WT1, CEA, HER-2/neu, MAGE-3, MAGE-4, MAGE-5, MAGE- 6, alpha-fetoprotein, CA 19-9, CA 72-4, NY-ESO, FAP, ErbB, c-Met, MART-1, CD30, EGFRvIII, immunoglobin kappa and lambda, CD38, CD52, CD3, CD4, CD8, CD5, CD7, CD2, and CD138 polypeptides.

In another embodiment, the target antigens include viral or fungal antigens, such as E6 and E7 from the human papillomavirus (HPV) or EBV (Epstein Barr virus) antigens; portions thereof; or surface exposed regions thereof.

In one embodiment, the TACI antigen recognition domain includes SEQ ID NO. 24.

In one embodiment, the BCMA antigen recognition domain includes SEQ ID NO. 25.

In one embodiment, the CS1 antigen recognition domain includes SEQ ID NO. 26.

In one embodiment, the BAFF-R antigen recognition domain includes SEQ ID NO. 27.

In one embodiment, the CD33 antigen recognition domain includes SEQ ID NO. 28.

In one embodiment, the CD123 antigen recognition domain includes SEQ ID NO. 29.

In one embodiment, the CD19 antigen recognition domain includes SEQ ID NO. 30.

In one embodiment, the CD20 antigen recognition domain includes SEQ ID NO. 31. In another embodiment, the CD20 antigen recognition domain includes SEQ ID NO. 32.

In one embodiment, the CD22 antigen recognition domain includes SEQ ID NO. 33.

In on embodiment, the CD45 antigen recognition domain includes SEQ ID NO. 34

The hinge region is a sequence positioned between for example, including, but not limited to, the chimeric antigen receptor, and at least one co-stimulatory domain and a signaling domain. The hinge sequence may be obtained including, for example, from any suitable sequence from any genus, including human or a part thereof. Such hinge regions are known in the art. In one embodiment, the hinge region includes the hinge region of a human protein including CD-8 alpha, CD28, 4-1BB, OX40, CD3-zeta, T cell receptor α or β chain, a CD3 zeta chain, CD28, CD3ε, CD45, CD4, CD5, CD8, CD8a, CD9, CD16, CD22, CD33, CD37, CD64, CD80, CD86, CD134, CD137, ICOS, CD154, functional derivatives thereof, and combinations thereof.

In one embodiment the hinge region includes the CD8 a hinge region.

In some embodiments, the hinge region includes one selected from, but is not limited to, immunoglobulin (e.g. IgG1, IgG2, IgG3, IgG4, and IgD).

The transmembrane domain includes a hydrophobic polypeptide that spans the cellular membrane. In particular, the transmembrane domain spans from one side of a cell membrane (extracellular) through to the other side of the cell membrane (intracellular or cytoplasmic).

The transmembrane domain may be in the form of an alpha helix or a beta barrel, or combinations thereof. The transmemebrane domain may include a polytopic protein, which has many transmembrane segments, each alpha-helical, beta sheets, or combinations thereof.

In one embodiment, the transmembrane domain that is naturally associated with one of the domains in the CAR is used. In another embodiment, the transmembrane domain is selected or modified by amino acid substitution to avoid binding of such domains to the transmembrane domains of the same or different surface membrane proteins to minimize interactions with other members of the receptor complex.

For example, a transmembrane domain includes a transmembrane domain of a T-cell receptor α or β chain, a CD3 zeta chain, CD28, CD3ε, CD45, CD4, CD5, CD7, CD8, CD9, CD16, CD22, CD33, CD37, CD64, CD80, CD86, CD68, CD134, CD137, ICOS, CD41, CD154, functional derivatives thereof, and combinations thereof.

In one embodiment, the transmembrane domain is artificially designed so that more than 25%, more than 50% or more than 75% of the amino acid residues of the domain are hydrophobic residues such as leucine and valine. In one embodiment, a triplet of phenylalanine, tryptophan and valine is found at each end of the synthetic transmembrane domain.

In one embodiment, the transmembrane domain is the CD8 transmembrane domain. In another embodiment, the transmembrane domain is the CD28 transmembrane domain. Such transmembrane domains are known in the art.

The signaling domain and co-stimulatory domain include polypeptides that provide activation of an immune cell to stimulate or activate at least some aspect of the immune cell signaling pathway.

In an embodiment, the signaling domain includes the polypeptide of a functional signaling domain of CD3 zeta, common FcR gamma (FCER1G), Fc gamma Rlla, FcR beta (Fc Epsilon Rib), CD3 gamma, CD3 delta, CD3 epsilon, CD79a, CD79b, DNAX-activating protein 10 (DAP10), DNAX-activating protein 12 (DAP12), active fragments thereof, functional derivatives thereof, and combinations thereof. Such signaling domains are known in the art.

In an embodiment, the CAR polypeptide further includes one or more co-stimulatory domains. In an embodiment, the co-stimulatory domain is a functional signaling domain from a protein including OX40; CD27; CD28; CD30; CD40; PD-1; CD2; CD7; CD258; Natural killer Group 2 member C (NKG2C); Natural killer Group 2 member D (NKG2D), B7-H3; a ligand that binds to at least one of CD83, ICAM-1, LFA-1 (CD1 la/CD18), ICOS, and 4-1BB (CD137); CDS; ICAM-1; LFA-1 (CD1a/CD18); CD40; CD27; CD7; B7-H3; NKG2C; PD-1; ICOS; active fragments thereof; functional derivatives thereof; and combinations thereof.

As used herein, the at least one co-stimulatory domain and signaling domain may be collectively referred to as the intracellular domain. As used herein, the hinge region and the antigen recognition may be collectively referred to as the extracellular domain.

The present disclosure further provides a polynucleotide encoding the chimeric antigen receptor polypeptide described above.

The term “polynucleotide” as used herein is defined as a chain of nucleotides. Polynucleotide includes DNA and RNA. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and polymerase chain reaction (PCR), and the like, and by synthetic means.

The polynucleotide encoding the CAR is easily prepared from an amino acid sequence of the specified CAR by any conventional method. A base sequence encoding an amino acid sequence can be obtained from the aforementioned NCBI RefSeq IDs or accession numbers of GenBenk for an amino acid sequence of each domain, and the nucleic acid of the present disclosure can be prepared using a standard molecular biological and/or chemical procedure. For example, based on the base sequence, a polynucleotide can be synthesized, and the polynucleotide of the present disclosure can be prepared by combining DNA fragments which are obtained from a cDNA library using a polymerase chain reaction (PCR).

In one embodiment, the polynucleotide disclosed herein is part of a gene, or an expression or cloning cassette.

The polynucleotide described above can be cloned into a vector. A “vector” is a composition of matter which includes an isolated polynucleotide and which can be used to deliver the isolated polynucleotide to the interior of a cell. Numerous vectors are known in the art including, but not limited to, linear polynucleotides, polynucleotides associated with ionic or amphiphilic compounds, plasmids, phagemid, cosmid, and viruses. Viruses include phages, phage derivatives. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, lentiviral vectors, and the like. In one embodiment, vectors include cloning vectors, expression vectors, replication vectors, probe generation vectors, integration vectors, and sequencing vectors.

In an embodiment, the vector is a viral vector. In an embodiment, the viral vector is a retroviral vector or a lentiviral vector. In an embodiment, the engineered cell is virally transduced to express the polynucleotide sequence.

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the patient either in vivo or ex vivo. A number of retroviral systems are known in the art. In some embodiments, adenovirus vectors are used. A number of adenovirus vectors are known in the art. In one embodiment, lentivirus vectors are used.

Viral vector technology is well known in the art and is described, for example, in Sambrook et al, (2001, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno- associated viruses, herpes viruses, and lentiviruses. In general, a suitable vector contains an origin of replication functional in at least one organism, a promoter sequence, convenient restriction endomiclease sites, and one or more selectable markers, (e.g., WO 01/96584; WO 01/29058; and U.S. Pat. No. 6,326,193).

Lentiviral vectors have been well known for their capability of transferring genes into human T cells with high efficiency but expression of the vector-encoded genes is dependent on the internal promoter that drives their expression. A strong promoter is particularly important for the third or fourth generation of CARs that bear additional co-stimulatory domains or genes encoding proliferative cytokines as increased CAR body size does not guarantee equal levels of expression. There are a wide range of promoters with different strength and cell-type specificity. Gene therapies using CAR T cells rely on the ability of T cells to express adequate CAR body and maintain expression over a long period of time. The EF-1α promoter has been commonly selected for the CAR expression.

The present invention relates to an expression vector containing a strong promoter for high level gene expression in T cells or NK cells. In further embodiment, the inventor discloses a strong promoter useful for high level expression of CARs in T cells or NK cells. In particular embodiments, a strong promoter relates to the SFFV promoter, which is selectively introduced in an expression vector to obtain high levels of expression and maintain expression over a long period of time in T cells or NK cells. Expressed genes prefer CARs, T cell co-stimulatory factors and cytokines used for immunotherapy.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor - 1 a (EF- 1 a). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the disclosure should not be limited to the use of constitutive promoters, inducible promoters are also contemplated as part of the disclosure. The use of an inducible promoter provides a molecular switch capable of turning on expression of the polynucleotide sequence, which is operatively linked when such expression is desired, or turning off the expression when expression is not desired. Examples of inducible promoters include, but are not limited to a metalothionine promoter, a glucocorticoid promoter, a progesterone promoter, and a tetracycline promoter.

Expression of chimeric antigen receptor polynucleotide may be achieved using, for example, expression vectors including, but not limited to, at least one of a SFFV (spleen-focus forming virus) (for example, SEQ ID NO. 23) or human elongation factor 11α (EF) promoter, CAG (chicken beta-actin promoter with CMV enhancer) promoter human elongation factor 1α (EF) promoter. Examples of less-strong/ lower-expressing promoters utilized may include, but is not limited to, the simian virus 40 (SV40) early promoter, cytomegalovirus (CMV) immediate-early promoter, Ubiquitin C (UBC) promoter, and the phosphoglycerate kinase 1 (PGK) promoter, or a part thereof. Inducible expression of chimeric antigen receptor may be achieved using, for example, a tetracycline responsive promoter, including, but not limited to, TRE3GV (Tet-response element, including all generations and preferably, the 3rd generation), inducible promoter (Clontech Laboratories, Mountain View, CA) or a part or a combination thereof.

In a preferred embodiment, the promoter is an SFFV promoter or a derivative thereof. It has been unexpectedly discovered that SFFV promoter provides stronger expression and greater persistence in the transduced cells in accordance with the present disclosure.

“Expression vector” refers to a vector including a recombinant polynucleotide comprising expression control sequences operatively linked to a nucleotide sequence to be expressed. An expression vector includes sufficient cis- acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide. The expression vector may be a bicistronic or multicistronic expression vectors. Bicistronic or multicistronic expression vectors may include (1) multiple promoters fused to each of the open reading frames;(2) insertion of splicing signals between genes; fusion of genes whose expressions are driven by a single promoter;(3) insertion of proteolytic cleavage sites between genes (self-cleavage peptide); and (iv) insertion of internal ribosomal entry sites (IRESs) between genes.

In one embodiment, the disclosure provides an engineered cell having at least one chimeric antigen receptor polypeptide or polynucleotide.

An “engineered cell” means any cell of any organism that is modified, transformed, or manipulated by addition or modification of a gene, a DNA or RNA sequence, or protein or polypeptide. Isolated cells, host cells, and genetically engineered cells of the present disclosure include isolated immune cells, such as NK cells and T cells that contain the DNA or RNA sequences encoding a chimeric antigen receptor or chimeric antigen receptor complex and express the chimeric receptor on the cell surface. Isolated host cells and engineered cells may be used, for example, for enhancing an NK cell activity or a T lymphocyte activity, treatment of cancer, and treatment of infectious diseases.

In an embodiment, the engineered cell includes immunoregulatory cells. Immunoregulatory cells include T-cells, such as CD4 T-cells (Helper T-cells), CD8 T-cells (Cytotoxic T-cells, CTLs), and memory T cells or memory stem cell T cells. In another embodiment, T-cells include Natural Killer T-cells (NK T-cells).

In an embodiment, the engineered cell includes Natural Killer cells. Natural killer cells are well known in the art. In one embodiment, natural killer cells include cell lines, such as NK-92 cells. Further examples of NK cell lines include NKG, YT, NK-YS, HANK-1, YTS cells, and NKL cells.

NK cells mediate anti-tumor effects without the risk of GvHD and are short-lived relative to T-cells. Accordingly, NK cells would be exhausted shortly after destroying cancer cells, decreasing the need for an inducible suicide gene on CAR constructs that would ablate the modified cells.

In accordance with the present disclosure, it was surprisingly found that NK cells provide a readily available cell to be engineered to contain and express the chimeric antigen receptor polypeptides disclosed herein.

Allogeneic or autologous NK cells induce a rapid immune response but disappear relatively rapidly from the circulation due to their limited lifespan. Thus, applicants surprisingly discovered that there is reduced concern of persisting side effects using CAR cell based therapy.

The disclosure includes a method of generating a cCAR. In some embodiments, the cCAR is generated using T-cells. In other embodiments, cCAR is using primary NK cells isolated from the peripheral blood or cord blood and NK-92 cells, such that they are administered “off-the-shelf” to any mammal with a disease or cancer.

According to one aspect of the present invention, NK cells can be expanded and transfected with CAR polynucleotides in accordance to the present invention. NK cells can be derived from cord blood, peripheral blood, iPS cells and embryonic stem cells. According to one aspect of the present invention, NK-92 cells may be expanded and transfected with CAR. NK-92 is a continuously growing cell line that has features and characteristics of natural killer (NK) cells (Arai, Meagher et al. 2008). NK-92 cell line is IL-2 dependent and has been proven to be safe(Arai, Meagher et al. 2008) and feasible. CAR expressing NK-92 cells can be expanded in the serum free-medium with or without co-culturing with feeder cells. A pure population of NK-92 carrying the CAR of interest may be obtained by sorting.

In one embodiment, engineered cells include allogeneic T cells obtained from donors that are modified to inactivate components of TCR (T cell receptor) involved in MHC recognition. As a result, TCR deficient T cells would not cause graft versus host disease (GVHD).

In some embodiments, the engineered cell may be modified to prevent expression of cell surface antigens. For example, an engineered cell may be genetically modified to delete the native CD45 gene to prevent expression and cell surface display thereof.

In some embodiments, the engineered cell includes an inducible suicide gene (“safety switch”) or a combination of safety switches, which may be assembled on a vector, such as, without limiting, a retroviral vector, lentiviral vector, adenoviral vector or plasmid. Introduction of a “safety switch” greatly increases safety profile and limits on-target or off-tumor toxicities of the compound CARs. The “safety switch” may be an inducible suicide gene, such as, without limiting, caspase 9 gene, thymidine kinase, cytosine deaminase (CD) or cytochrome P450. Other safety switches for elimination of unwanted modified T cells involve expression of CD20 or CD19 or truncated epidermal growth factor receptor in T cells. All possible safety switches are have been contemplated and are embodied in the present invention.

In some embodiments, the suicide gene is integrated into the engineered cell genome.

In one embodiment, the present disclosure provides an engineered cell having a CD45 chimeric antigen receptor polynucleotide. In one embodiment, the CD45 CAR polypeptide includes SEQ ID NO. 13 and corresponding polynucleotide sequence SEQ ID NO. 14. In another embodiment, the CD45 CAR polypeptide includes SEQ ID NO. 15, and corresponding polynucleotide sequence SEQ ID NO. 16. In another embodiment, the CD45 CAR polypeptide includes SEQ ID NO. 17, and corresponding polynucleotide sequence SEQ ID NO. 18.

### Multiple CAR Units

The present disclosure provides an engineered cell having at least two distinct CAR polypeptides.

As used herein, compound CAR (cCAR) or multiple CAR refers to an engineered cell having at least two distinct chimeric antigen receptor polypeptides. As used herein, a “distinct chimeric antigen receptor polypeptide” has a unique antigen recognition domain, a signal peptide, a hinge region, a transmembrane domain, at least one costimulatory domain, and a signaling domain. Therefore, two unique chimeric antigen receptor polypeptides will have different antigen recognition domains. The signal peptide, hinge region, transmembrane domain, at least one costimulatory domain, and signaling domain may be the same or different between the two distinct chimeric antigen receptor polypeptides. As used herein, a chimeric antigen receptor (CAR) unit refers to a distinct chimeric antigen receptor polypeptide, or a polynucleotide encoding for the same.

As used herein, a unique antigen recognition domain is one that is specific for or targets a single target, or a single epitope of a target.

In some embodiments, the compound CAR targets the same antigen. For example, cCAR targets different epitopes or parts of a single antigen. In some embodiments, each of the CAR units present in the compound CAR targets different antigen specific to the same or different disease condition or side effects caused by a disease condition.

In some embodiments, the compound CAR targets two different antigens.

Creation of compound CARs bearing different CAR units can be quiet challenging: (1) CAR-CAR interactions might have a deleterious effect and an appropriate CAR design is a key to offset this effect; (2) a compound CAR in a single construct could increase the length of the expression cassette, which may cause the reduction of the viral titer and level of protein expression; (3) an appropriate design to include various CAR body elements particularly to select a strategy to express multiple CARs in a single vector is required; (4) A strong promoter is particularly important for a compound CAR that bears additional units of CAR; (5) The hinge region in the CAR needs to is designed so that interaction of the hinge region between each CAR unit is avoided preferably; (6) two or more units of CARs expressing in a cell may cause toxic effects (CAR-CAR interaction). Applicants herein provide a novel and surprising CAR compositions and methods to overcome these hurdles.

In one embodiment, the present disclosure provides an engineered cell having multiple CAR units. This allows a single engineered cell to target multiple antigens. Targeting multiple surface markers or antigens simultaneously with a multiple CAR units prevents selection of resistant clones and reduces tumor recurrence. Multiple CAR T cell immunotherapies, with each individual component CAR comprising various domains and activation sites has not yet been developed for any malignancies.

In one aspect of the present invention, cCAR includes multiple CAR units. In some embodiments, cCAR includes at least two CAR units. In another embodiment, the cCAR includes at least three CAR units. In another embodiment, the cCAR includes at least four units.

In one embodiment, the present disclosure provides an engineered cell having at least two distinct chimeric antigen receptor polypeptides, each having a different antigen recognition domain.

In a preferred embodiment, the engineered cell having at least two distinct chimeric antigen receptor polypeptides is a primary NK cells isolated from the peripheral blood or cord blood and NK-92 cells, such that they are administered “off-the-shelf” to any mammal with a disease or cancer.

In one embodiment, the engineered cell includes (i.) a first chimeric antigen receptor polypeptide comprising a first antigen recognition domain, a first signal peptide, a first hinge region, a first transmembrane domain, a first co-stimulatory domain, and a first signaling domain; and (ii.) a second chimeric antigen receptor polypeptide comprising a second antigen recognition domain, a second signal peptide, a second hinge region, a second transmembrane domain, a second co-stimulatory domain, and a second signaling domain. The first antigen recognition domain is different from the second antigen recognition domain.

In a preferred embodiment, each engineered CAR unit polynucleotide have different nucleotide sequences in order to avoid homologous recombination.

In one embodiment, the target of the first antigen recognition domain is selected from the group consisting of interleukin 6 receptor, NY-ESO-1, alpha fetoprotein (AFP), glypican-3 (GPC3), BAFF-R, BCMA, TACI, LeY, CD5, CD13, CD14, CD15 CD19, CD20, CD22, CD33, CD41, CD61, CD64, CD68, CD117, CD123, CD138, CD267, CD269, CD38, Flt3 receptor, and CS1; and the target of the second recognition domain is selected from the group consisting of interleukin 6 receptor, NY-ESO-1, alpha fetoprotein (AFP), glypican-3 (GPC3), BAFF-R, BCMA, TACI, LeY, CD5, CD13, CD14, CD15 CD19, CD20, CD22, CD33, CD41, CD61, CD64, CD68, CD117, CD123, CD138, CD267, CD269, CD38, Flt3 receptor, and CS1.

In one embodiment, the engineered cell includes a first chimeric antigen receptor polypeptide having a CD19 antigen recognition domain and second chimeric antigen receptor polypeptide having a CD20 recognition domain. In one embodiment, this engineered cell includes a polypeptide of SEQ ID NO. 3 and corresponding polynucleotide of SEQ ID NO. 4.

In one embodiment, the engineered cell includes a first chimeric antigen receptor polypeptide having a CD19 antigen recognition domain and second chimeric antigen receptor polypeptide having a CD22 antigen recognition domain. In one embodiment, this engineered cell includes a polypeptide of SEQ ID NO. 5 and corresponding polynucleotide of SEQ ID NO. 6.

In one embodiment, the engineered cell includes a first chimeric antigen receptor polypeptide having a CD19 antigen recognition domain and second chimeric antigen receptor polypeptide having a CD123 antigen recognition domain. In one embodiment, this engineered cell includes a polypeptide of SEQ ID NO. 7 and corresponding polynucleotide of SEQ ID NO. 8.

In one embodiment, the engineered cell includes a first chimeric antigen receptor polypeptide having a CD33 antigen recognition domain and second chimeric antigen receptor polypeptide having a CD123antigen recognition domain. In one embodiment, this engineered cell includes a polypeptide of SEQ ID NO. 9 and corresponding polynucleotide of SEQ ID NO. 10. In another embodiment, this engineered cell includes a polypeptide of SEQ ID NO. 11 and corresponding polynucleotide of SEQ ID NO. 12.

In one embodiment, the engineered cell includes a first chimeric antigen receptor polypeptide having a BAFF-R antigen recognition domain and second chimeric antigen receptor polypeptide having a CS1antigen recognition domain.

In one embodiment, the engineered cell includes a first chimeric antigen receptor polypeptide having a CD269 antigen recognition domain and second chimeric antigen receptor polypeptide having a CS1 antigen recognition domain. In one embodiment, the engineered cell includes a polypeptide including SEQ ID NO. 19 and corresponding polynucleotide SEQ ID NO. 20. In one embodiment, the engineered cell includes a polpeptide including SEQ ID NO. 21 and corresponding polynucleotide SEQ ID NO. 22.

In one embodiment, the engineered cell includes a first chimeric antigen receptor polypeptide having a CD33 antigen recognition domain and second chimeric antigen receptor polypeptide having a CD123 antigen recognition domain.

In one embodiment, each CAR unit includes the same or different hinge region. In another embodiment, each CAR unit includes the same or different transmembrane region. In another embodiment, each CAR unit includes the same or different intracellular domain.

In one embodiment, each CAR unit includes the CD3 zeta chain signaling domain.

In one embodiment, each distinct CAR unit includes different co-stimulatory domains to avoid interaction. For example, the first chimeric antigen receptor polypeptide includes a 4-BB co-stimulatory domain; and the second chimeric antigen receptor polypeptide includes a CD28 co-stimulatory domain.

In another embodiment, the hinge region is designed to exclude amino acids that may cause undesired intra- or intermolecular interactions. For example, the hinge region may be designed to exclude or minimize cysteine residues to prevent formation of disulfide bonds. In another embodiment, the hinge region may be designed to exclude or minimize hydrophobic residues to prevent unwanted hydrophobic interactions.

Compound CAR can perform killing independently or in combination. Multiple or compound CAR comprises same or different hinge region, same or different transmembrane, same or different co-stimulatory and same or different intracellular domains. Preferably, the hinge region is selected to avoid the interaction site.

The compound CAR of the present invention may target same or different tumor populations in T or NK cells. The first CAR, for example, may target the bulky tumor population and the next or the second CAR, for example, may eradicate cancer or leukemic stem cells, to avoid cancer relapses.

In accordance with the present invention it was surprisingly found that the compound CAR in a T or NK cells targeting different or same tumor populations combat tumor factors causing cancer cells resistant to the CAR killing activity, thereby producing down regulation of the target antigen from the cancer cell surface. It was also surprisingly found that this enables the cancer cell to “hide” from the CAR therapy referred to as “antigen escape” and tumor heterogeneity, by which different tumor cells can exhibit distinct surface antigen expression profiles.

### Engineered Cell Having CAR Polypeptide and Enhancer

In another embodiment, the present disclosure provides an engineered cell having at least one chimeric antigen receptor polypeptide and an enhancer.

In one embodiment, the present disclosure provides an engineered cell having at least two distinct chimeric antigen receptor polypeptides and an enhancer.

As used herein, an enhancer includes a biological molecule that promotes or enhances the activity of the engineered cell having the chimeric antigen receptor polypeptide. Enhancers include cytokines. In another embodiment, enhancers include IL-2, IL-7, IL-12, IL-15, IL-21, PD-1, PD-L1, CSF1R, CTAL-4, TIM-3, and TGFR beta, receptors for the same, and functional fragments thereof.

Enhancers may be expressed by the engineered cell described herein and displayed on the surface of the engineered cell or the enhancer may be secreted into the surrounding extracellular space by the engineered cell. Methods of surface display and secretion are well known in the art. For example, the enhancer may be a fusion protein with a peptide that provides surface display or secretion into the extracellular space.

The effect of the enhancer may be complemented by additional factors such as enhancer receptors and functional fragments thereof. The additional factors may be co-expressed with the enhancer as a fusion protein or expressed as a separate peptide and secreted into the extracellular space.

In one embodiment, the enhancer is IL-15. In this instance, the additional factor is the IL-15 receptor, and functional fragments thereof. Functional fragments include the IL-15 receptor, IL-15RA, and the sushi domain of IL-15RA. An example of a suitable sushi domain includes SEQ ID NO. 35. In accordance with the present disclosure, any chimeric antigen receptor polypeptide disclosed herein includes the Human Interleukin 15 with human interleukin 2 signal peptide SEQ ID NO. 36.

Interleukin (IL)-15 and its specific receptor chain, IL-15Rα (IL-15-RA) play a key functional role in various effector cells, including NK and CD8 T cells. CD8+ T cells can be modified to express autocrine growth factors including, but not limited to, IL-2, Il-7, IL21 or IL-15, to sustain survival following transfer in vivo. Without wishing to be bound by theory, it is believed that IL-15 could overcome the CD4 deficiency to induce primary and recall memory CD8T cells. Overexpression of IL-15-RA or an IL-15 IL-RA fusion on CD8 T cells significantly enhances its survival and proliferation in-vitro and in-vivo. In some embodiments, CD4CAR or any CAR can include expressing any one or more of moieties, IL-15, IL15RA and IL-15/IL-15R or IL15-RA/IL-15, or a part or a combination thereof, to enhance survival or proliferation of CAR T or NK, and to improve expansion of memory CAR CD8+ T cells.

The present disclosure relates to an engineered cell having a CAR as described herein and any one or more of moieties of IL-15, IL15RA and IL-15/IL-15R or IL15-RA/IL-15, or a part or a combination thereof, to enhance survival or persistent or proliferation of CAR T or NK for treating cancer in a patient.

In one embodiment, the engineered cell includes a CD4 chimeric antigen receptor polypeptide and IL-15RA (SEQ ID NO. 1), and corresponding polynucleotide (SEQ ID NO. 2).

### Methods of Generating Engineered Cells

Any of the polynucleotides disclosed herein may be introduced into an engineered cell by any method known in the art.

In one embodiment, CAR polynucleotides are delivered to the engineered cell by any viral vector as disclosed herein.

In one embodiment, to achieve enhanced safety profile or therapeutic index, the any of the engineered cells disclosed herein be constructed as a transient RNA-modified “biodegradable” version or derivatives, or a combination thereof. The RNA-modified CARs of the present invention may be electroporated into T cells or NK cells. The expression of the compound CAR may be gradually diminished over few days.

In some embodiments of the present invention, any of the engineered cells disclosed herein may be constructed in a transponson system (also called a “Sleeping Beauty”), which integrates the CAR DNA into the host genome without a viral vector.

### Methods of Generating an Engineered Cell Having Multiple CAR Units

In another embodiment, the present disclosure provides a method making an engineered cell having at least two CAR units.

In some embodiments, multiple units of CAR are expressed in a T or NK cell using bicistronic or multicistronic expression vectors. There are several strategies can be employed to construct bicistronic or multicistronic vectors including, but not limited to, (1) multiple promoters fused to the CARs′ open reading frames;(2) insertion of splicing signals between units of CAR; fusion of CARs whose expressions are driven by a single promoter;(3) insertion of proteolytic cleavage sites between units of CAR (self-cleavage peptide); and (iv) insertion of internal ribosomal entry sites (IRESs).

In a preferred embodiment, multiple CAR units are expressed in a single open reading frame (ORF), thereby creating a single polypeptide having multiple CAR units. In this embodiment, an amino acid sequence or linker containing a high efficiency cleavage site is disposed between each CAR unit.

As used herein, high cleavage efficiency is defined as more than 50%, more than 70%, more than 80%, or more than 90% of the translated protein is cleaved. Cleavage efficiency may be measured by Western Blot analysis, as described by Kim 2011.

Furthermore, in a preferred embodiment, there are equal amounts of cleavage product, as shown on a Western Blot analysis.

Examples of high efficiency cleavage sites include porcine teschovirus-1 2A (P2A), FMDV 2A (abbreviated herein as F2A); equine rhinitis A virus (ERAV) 2A (E2A); and Thoseaasigna virus 2A (T2A), cytoplasmic polyhedrosis virus 2A (BmCPV2A) and flacherie Virus 2A (BmIFV2A), or a combination thereof. In a preferred embodiment, the high efficiency cleavage site is P2A. High efficiency cleavage sites are described in Kim JH, Lee S-R, Li L-H, Park H-J, Park J-H, Lee KY, et al. (2011) High Cleavage Efficiency of a 2A Peptide Derived from Porcine Teschovirus-1 in Human Cell Lines, Zebrafish and Mice. PLoS ONE 6(4): e18556, the contents of which are incorporated herein by reference.

In embodiments wherein multiple CAR units are expressed in a single open reading frame (ORF), expression is under the control of a strong promoter. Examples of strong promoters include the SFFV promoter, and derivatives thereof.

### Engineered Cell Having CAR Polypeptide and Enhancer

In another embodiment, the present disclosure provides a method making an engineered cell that expresses at least one CAR unit and an enhancer.

In some embodiments, at least one CAR unit and enhancer is expressed in a T or NK cell using bicistronic or multicistronic expression vectors. There are several strategies can be employed to construct bicistronic or multicistronic vectors including, but not limited to, (1) multiple promoters fused to the CARs′ open reading frames;(2) insertion of splicing signals between units of CAR; fusion of CARs whose expressions are driven by a single promoter;(3) insertion of proteolytic cleavage sites between units of CAR (self-cleavage peptide); and (iv) insertion of internal ribosomal entry sites (IRESs).

In a preferred embodiment, at least one CAR unit and an enhancer are expressed in a single open reading frame (ORF), thereby creating a single polypeptide having at least one CAR unit and an enhancer. In this embodiment, an amino acid sequence or linker containing a high efficiency cleavage site is disposed between each CAR unit and between a CAR unit and enhancer. In this embodiment, the ORF is under the control of a strong promoter. Examples of strong promoters include the SFFV promoter, and derivatives thereof.

Furthermore, in a preferred embodiment, there are equal amounts of cleavage product, as shown on a Western Blot analysis.

### Methods of Treatment Using the Compositions Disclosed Herein

In another embodiment, the present invention provides a method of targeting CD45 for conditioning prior to allogenic transplantation in cancer treatment. CD45 is also known as leukocyte common antigen (LCA) and is a tyrosine phosphatase expressed on virtually all cells of hematopoietic origin except erythrocytes and platelets. Most hematologic malignancies express CD45. For instance, 85% to 90% acute lymphoid and myeloid leukemias express CD45. CD45 is not found in non-hematopoietic origin. In addition, CD45 is expressed at a high density of an average copy number of approximately 200,000 molecules per cells on malignant cells and leukocytes. CD45 presents an ideal target for a variety of hematologic malignancies. However, CAR T and NK cells also express CD45. Without inactivation of endogenous CD45, CAR T or NK cells armed with CARs targeting CD45 may result in self-killing.

The association of CD45 with TCR complexes is essential in regulation of T-cell activation in response to antigen. The inability of CD45-deficient T cells to present antigen is due to reduced signaling through the T cell receptors (TCRs). TCRs are cell surface receptors that play an essential role in the activation of T cells in response to the presentation of antigen. The TCR is generally made from two chains, alpha and beta, which are associated with the transducing subunits, the CD3, to form the T-cell receptor complex present on the cell surface.

It was surprisingly found that multiple CARs (Compound CARs, cCAR) of the present invention combat a key mechanism by which cancer cells resist CAR activity, i.e., the downregulation or heterogeneous expression of the target antigen from the cancer cell surface. This mechanism allows the cancer cell to “hide” from the CAR therapy, a phenomenon referred to as ‘antigen escape’. The present disclosure pre-empts cancer antigen escape by recognizing a combination of two or more antigens to rapidly eliminate the tumor.

The invention provides a method of simultaneous targeting of multi-antigens using a cCAR resulting in improved tumor control by minimizing the possibility of tumor selection on the basis of target antigen loss or down-regulation.

The disclosed invention includes compound (multiple or compound) cCAR in a T or NK cell targeting different or same surface antigens present in tumor cells. The compound chimeric antigen receptors of present invention comprise at least multiple chimeric receptor constructs linked by a linker and target same or different antigens. For example, each of the CAR construct present in the compound CAR (cCAR) construct includes an antigen recognition domain, an extracellular domain, a transmembrane domain and/or a cytoplasmic domain. The extracellular domain and transmembrane domain can be derived from any desired source for such domains. The multiple CAR constructs are linked by a linker. The expression of the compound CAR construct is driven by a promoter. The linker may be a peptide or a part of a protein, which is self-cleaved after a protein or peptide is generated (also called as a self-cleaving peptide).

In one embodiments, the compound CARs of the present invention target Myelodysplastic Syndrome and acute myeloid leukemia (AML) opulation. Myelodysplastic Syndrome (MDS) remains an incurable hematopoietic stem cell malignancy that occurs most frequently among the elderly, with about 14,000 new cases each year in the USA. About 30-40% of MDS cases progress to AML. The incidence of MDS continues to increase as our population ages. Although MDS and AML have been studied intensely, no satisfactory treatments have been developed.

The compositions and methods of this invention can be used to generate a population of T lymphocyte or NK cells that deliver both primary and co-stimulatory signals for use in immunotherapy in the treatment of cancer, in particular, the treatment of lung cancer, melanoma, breast cancer, prostate cancer, colon cancer, renal cell carcinoma, ovarian cancer, brain cancer, sarcoma, leukemia and lymphoma.

Immunotherapeutics generally rely on the use of immune effector cells and molecules to target and destroy cancer cells. The effector may be a lymphocyte carrying a surface molecule that interacts, either directly or indirectly, with a tumor cell target. Various effector cells include cytotoxic T cells, NK cells and NK-92 cells. The compositions and methods described in the present invention may be utilized in conjunction with other types of therapy for cancer, such as chemotherapy, surgery, radiation, gene therapy, and so forth. The compositions and methods described in the present invention may be utilized in other disease conditions that rely on immune responses such as inflammation, immune diseases, and infectious diseases.

In some embodiments, the compound CAR of the present invention may act as a bridge to bone marrow transplant, by achieving complete remission for patients who have minimal residual disease and are no longer responding to chemotherapy. In other embodiments, the compound CAR eliminates leukemic cells followed by bone marrow stem cell rescue to support leukopenia.

In some embodiments, the compound CAR of the present disclosure can combat a key mechanism by which cancer cells resist CAR activity by the down-regulation of the target antigen. In another embodiment, the invented compound CAR can also combat the heterogeneity of cancer cells, which creates significant challenges in a regular CAR T/NK cell therapy. In a further embodiment, the disclosed compound CAR is designed that the first CAR targets the bulky tumor population and another eradicates cancer or leukemic stem cells to avoid cancer relapses.

In one embodiment, the present disclosure provides a method of destroying cells having a CD33 antigen or a CD123 antigen, or both by contacting said cells with an engineered cell having at least one of chimeric antigen receptor polypeptide having a CD33 antigen recognition domain and chimeric antigen receptor polypeptide having a CD23 antigen recognition domain. The engineered cell may be a T or NK cell.

Cells having at least one of the CD33 antigen and the CD123 antigen include acute myeloid leukemia, precursor acute lymphoblastic leukemia, chronic myeloproliferative neoplasms, chronic myeloid leukemia, myelodysplasia syndromes, blastic plasmocytoid dendritic neoplasms (BPDCN), Hodgkin’s lymphoma, mastocytosis, and hairy cell leukemia cells.

In another embodiment, the present disclosure provides a method of providing myeloblative conditioning regimens for hematopoietic stem cell transplantation. In this embodiment, a T or NK engineered cell having a CD33 unit and a CD123 unit is administered to a patient in need thereof.

In further embodiments, the present disclosure provides a method of eradicating or killing leukemic stem cells (LSCs) or bulk leukemic cells expressing CD123 or CD33, or both. In this embodiment, a T or NK engineered cell having a CD33 unit and a CD123 unit is administered to a patient in need thereof.

In further embodiments, the compound CAR in a T or NK cell may be used to eradicate or kill CD34+ CD38- leukemic stem cells or bulk leukemic cells expressing CD123 or CD33 or both.

In some embodiments, a compound CAR targets cells expressing CD19 or CD20 antigens or both. In another embodiment, a compound CAR targets cells expressing CD19 or CD22 antigens or both. The targeted cells may be cancer cells, such as, without limiting, B-cell lymphomas or leukemias. In further embodiments, the target antigens can include at least one of this group, but not limited to, ROR1, PSMA, MAGE A3, Glycolipid, glypican 3, F77, GD-2, WT1, CEA, HER-2/neu, MAGE-3, MAGE-4, MAGE-5, MAGE-6, alpha-fetoprotein, CA 19-9, CA 72-4, NY-ESO, FAP, ErbB, c-Met, MART-1, CD30, EGFRvIII, immunoglobin kappa and lambda, CD38, CD52, CD3, CD4, CD8, CD5, CD7, CD2, and CD138. The target antigens can also include viral or fungal antigens, such as E6 and E7 from the human papillomavirus (HPV) or EBV (Epstein Barr virus) antigens.

In some embodiments, the compound CAR targets cells expressing CD19 or CD123 antigen or both. The targeted cells are cancer cells, such as, without limiting, B-cell lymphomas or leukemias.

In further embodiments, the compound CAR targets cells expressing CS1 and/or B-cell maturation antigens (BCMA) or both. In another embodiment, the targeting cells are malignant plasma cells, such as, without limiting, multiple myeloma.

In some embodiments, the compound CAR targets cells expressing multiple antigens including, but not limited to, CS1, BCMA, CD267, BAFF-R, CD38, CD138, CD52, CD19, CD20, interleukin 6 receptor and NY-ESO-1 antigens. In another embodiment, the targeting cells are malignant plasma cells such as, without limiting, multiple myeloma.

In some embodiments, the compound CAR targets cells expressing multiple antigens including but not limited to, alpha fetoprotein (AFP) and Glypican-3 (GPC3). In another embodiment, the targeting cells are hepatocellular carcinoma, fibrolamellar carcinoma, hepatoblastoma, undifferentiated embryonal sarcoma and mesenchymal hamartoma of liver, lung-squamous cell carcinoma, testicular nonseminomatous germ cell tumors, liposarcoma, ovarian and extragonadal yolk sac tumors, ovarian choriocarcinoma, teratomas, ovarian clear cell carcinoma, and placental site trophoblastic tumor.

In accordance with the present invention, the T or NK cell comprising compound CARs targeting different or same antigens offset tumor escape and enables simultaneous targeting of tumor cells.

The T or NK host cells comprising compound CAR disclosed herein is embodied in the present disclosure. The nucleotide and polypeptide constructs, sequences, host cells, vectors of the compound CAR is considered to be part of the present disclosure and is embodied herein.

In some embodiments, the compound CAR is administrated in combination with any chemotherapy agents currently being developed or available in the market. In some embodiments, the compound CAR is administrated as a first line treatment for diseases including, but not limited to, hematologic malignancies, cancers, non-hematologic malignances, inflammatory diseases, infectious diseases such as HIV and HTLV and others. In one embodiment, T cells expressing the compound CAR are co-administrated with NK cells expressing the same or different compound CAR as an adaptive immunotherapy. Compound CAR NK cells provide rapid, innate activity targeting cells while compound T cells provide relative long-lasting adaptive immune activity.

In one embodiment, the cells expressing a compound CAR are administrated as a bridge to bone marrow stem transplantation for mammals, e.g. patients who are resistant to chemotherapies and are not qualified for bone marrow stem cell transplantation.

In some embodiments, the compound CAR co-expresses a transgene and releases a transgenic product, such as IL-12 in the targeted tumor lesion and further modulates the tumor microenvironment.

In one embodiment, cells expressing a compound CAR are administrated to a mammal for bone marrow myeloid ablation as a part of the treatment to a disease.

In a specific embodiment, the cells expressing a compound CAR can be T cells or NK cells, administrated to a mammal, e.g. human. The presented disclosure includes a method of treating a mammal having a disorder or disease by administration of a compound CAR. The targeted cells may be cancer cells such as, or cells affected by any other disease condition, such as infectious diseases, inflammation, and autoimmune disorders.

The present invention is intended to include the use of fragments, mutants, or variants (e.g., modified forms) of the compound CAR or antigens that retain the ability to induce stimulation and proliferation of T/NK cells. A “form of the protein” is intended to mean a protein that shares a significant homology with at least one CAR or antigen and is capable of effecting stimulation and proliferation of T/NK cells. The terms “biologically active” or “biologically active form of the protein,” as used herein, are meant to include forms of the proteins or variants that are capable of effecting anti-tumor activity of the cells.

The compositions and methods of this invention can be used to generate a population of T/NK cells that deliver both primary and co-stimulatory signals for use in immunotherapy in the treatment of cancer, in particular the treatment of lung cancer, melanoma, breast cancer, prostate cancer, colon cancer, renal cell carcinoma, ovarian cancer, neuroblastoma, rhabdomyosarcoma, leukemia and lymphoma. The compositions and methods described in the present invention may be utilized in conjunction with other types of therapy for cancer, such as chemotherapy, surgery, radiation, gene therapy, and so forth.

In some embodiments, the invention discloses a method of depletion B cells, immature B cells, memory B cells, plasmablasts, long lived plasma cells, or plasma cells in patients with an autoimmune disease by administering to patients with CAR or compound CAR T cells or NK cells. CAR targeted cells are B or plasma cells expressing one or two or all of antigens, BCMA, TACI and BAFF-R. The autoimmune diseases include systemic scleroderma, multiple sclerosis, psoriasis, dermatitis, inflammatory bowel diseases (such as Crohn’s disease and ulcerative colitis), systemic lupus erythematosus, vasculitis, rheumatoid arthritis, Sjorgen’s syndrome, polymyositis, granulomatosis and vasculitis, Addison’s disease, antigen-antibody complex mediated diseases and anti-glomerular basement membrane disease.

The present invention relates to methods of treating or managing a subject having an autoimmune disorder or organ rejection utilizing CAR that binds to T-cell surface antigen, but not limited to CD2, CD3, CD4, CD5, CD7 and an antigen that is present on the surface of B-cells or Plasma cells, but not limited to CD19, CD20, CD22, CD38, CS1 (CD319, SLAMF7) and BCMA. In preferred embodiment, the dual CAR where the one CAR unit targets for T-cells by selecting at least one of the target antigens from the following group: CD2, CD3, CD4, CD5, and CD7; and the other CAR unit targets for B-cells by selecting at least one of the target antigens from the following group: CD19, CD20, CD22; or plasma cells by selecting at least one of the target antigens from the following group or target antigen BCMA, CD38, CS1 (CD319. SLAMF7) and CD138.

In another embodiment, the dual CAR can be a tandem CAR, compound CAR, cistronic chimeric antigen receptor CAR and bispecific CAR.

The structure of compound CAR (cCAR) and methods of generating cCAR are described in PCT/US2016/039306, PCT/US2016/068349, PCT/US2018/038529

In one embodiment, the present invention provides a method of treating or managing an autoimmune disorder, the method whereby a CAR comprised by selecting one of the target antigens from the following group: CD2, CD3, CD4, CD5, or CD7. In further embodiments, the CAR T-cells deplete autoreactive T-cells against healthy host tissues.

The term “autoimmune disease” as used herein is defined as a disorder that results from an autoimmune response. An autoimmune disease is the result of an inappropriate and excessive response to a self-antigen. Examples of autoimmune diseases include but are not limited to, achalasia, Addison’s disease, acute inflammatory demyelinating polyneuropathy - AIDP, adult Still’s disease, agammaglobulinemia, alopecia areata, amyloidosis, ankylosing spondylitis, anti-GBM/anti-TBM nephritis, anti-PAD4-activating rheumatoid arthritis, antiphospholipid syndrome, asthma, atopic dermatitis, autoimmune angioedema, autoimmune dysautonomia, autoimmune encephalomyelitis, autoimmune hepatitis, autoimmune inner ear disease (AIED), autoimmune myocarditis, autoimmune oophoritis, autoimmune orchitis, autoimmune pancreatitis, autoimmune retinopathy, autoimmune thrombocytopenia, autoimmune urticarial, axonal & neuronal neuropathy (AMAN). Balo disease, Behcet’s disease, benign mucosal pemphigoid, bullous pemphigoid, Castleman disease (CD), celiac disease, Chagas disease, chronic inflammatory demyelinating polyneuropathy (CIDP), chronic recurrent multifocal osteomyelitis (CRMO), Churg-Strauss Syndrome (CSS) or eosinophilic granulomatosis (EGPA), cicatricial pemphigoid, Cogan’s syndrome, cold agglutinin disease, congenital heart block, coxsackie myocarditis, CREST syndrome, Crohn’s disease, dermatitis, dermatitis herpetiformis, dermatomyositis, Devic’s disease (neuromyelitis optica), diabetes mellitus, discoid lupus, Dressier’s syndrome, endometriosis, eosinophilic esophagitis (EoE), eosinophilic fasciitis, erythema nodosum, essential mixed cryoglobulinemia, Evans syndrome, fibromyalgia, fibrosing alveolitis, giant cell arteritis (temporal arteritis), giant cell myocarditis, Goodpasture’s syndrome, granulomatosis with polyangiitis. Graves’ disease, Guillain-Barre syndrome, Hashimoto’s disease, Hashimoto’s thyroiditis, autoimmune hemolytic anemia, Henoch-Schonlein purpura (HSP), herpes gestationis or pemphigoid gestationis (PG), Hidradenitis Suppurativa (HS) (Acne Inversa), hypogammalglobulinemia, idiopathic membranous nephropathy, idiopathic thrombocytopenic purpura. IgA nephropathy, IgG4-related disease, IgG4-related sclerosing disease, IgG neuropathy, IgM polyneuropathy, immune thrombocytopenic purpura (ITP), inclusion body myositis (IBM), inflammatory bowel disease (IBD), interstitial cystitis (IC), juvenile arthritis, juvenile diabetes (type 1 diabetes), juvenile myositis (JM), Kawasaki disease, Lambert-Eaton syndrome, leukocytoclastic vasculitis, Lichen planus, Lichen sclerosus, ligneous conjunctivitis, linear IgA disease (LAD), lupus, lyme disease chronic, membranous nephropathy, Meniere’s disease, microscopic polyangiitis (MPA), mixed connective tissue disease (MCTD), Mooren’s ulcer, Mucha-Habermann disease, multifocal motor neuropathy (MMN) or MMNCB, multiple sclerosis, myasthenia gravis, myositis, narcolepsy, neonatal lupus, neuromyelitis optica, neutropenia, ocular cicatricial pemphigoid, optic neuritis, palindromic rheumatism (PR), PANDAS, paraneoplastic cerebellar degeneration (PCD), paroxysmal nocturnal hemoglobinuria (PNH), Parry Romberg syndrome, pars planitis (peripheral uveitis), Parsonage-Turner syndrome, pemphigus, pemphigus vulgaris, pemphigus foliaceus, peripheral neuropathy, perivenous encephalomyelitis, pernicious anemia (PA), POEMS syndrome, polyarteritis nodosa, polyglandular syndromes types I, II, and III, polymyalgia rheumatic, polymyositis, postmyocardial infarction syndrome, postpericardiotomy syndrome, primary biliary cirrhosis, primary sclerosing cholangitis, progesterone dermatitis, psoriasis, psoriatic arthritis, pure red cell aplasia (PRCA), pyoderma gangrenosum, Raynaud’s syndrome, reactive Arthritis, reflex sympathetic dystrophy, relapsing polychondritis, restless legs syndrome (RLS), retroperitoneal fibrosis, rheumatic fever, rheumatoid arthritis, juvenile rheumatoid arthritis, sarcoidosis, Schmidt syndrome, scleritis, scleroderma, sensitized / preformed antibodies in solid organ transplant, Sjogren’s syndrome, sperm & testicular autoimmunity, stiff person syndrome (SPS), systemic lupus erythematosus (SLE), subacute bacterial endocarditis (SBE), Susac’s syndrome, sympathetic ophthalmia (SO), Takayasu’s arteritis, temporal arteritis/Giant cell arteritis, thrombocytopenic purpura, thrombotic thrombocytopenic purpura (TTP). Tolosa-Hunt syndrome (THS), transverse myelitis, type 1 diabetes, ulcerative colitis (UC), undifferentiated connective tissue disease (UCTD), uveitis, vasculitis, vitiligo, Vogt-Koyanagi-Harada disease; and Wegener’s disease. Guillain-Barr syndrome, Hashimoto’s disease, hemolytic anemia, systemic lupus erythematosus, multiple sclerosis, N-methyl-D-aspartate receptor (NMDAR) encephalitis, myelin-oligodendrocyte glycoprotein (MOG) spectrum disorders (MOGSD), neuromyelitis optica spectrum (NMOSD), myasthenia gravis, pemphigus vulgaris, psoriasis, rheumatic fever, rheumatoid arthritis, sarcoidosis, scleroderma, Sjogren’s syndrome, spondyloarthropathies, thyroiditis, vasculitis, vitiligo, myxedema, pernicious anemia, and ulcerative colitis. In preferred embodiments, the autoimmune disorder is not IgG4-related disease. In preferred embodiments, the autoimmune disorder is AAV (e.g. relapsed or refractory AAV), SLE (e.g. relapsed or refractory SLE), or rheumatoid arthritis (e.g. relapsed or refractory rheumatoid arthritis). In particularly preferred embodiments, the autoimmune disorder is AAV (e.g. relapsed or refractory AAV) or rheumatoid arthritis (e.g. relapsed or refractory rheumatoid arthritis).

In preferred embodiments, the autoimmune disorder is caused by T lineage cells (e.g. autoreactive T lineage cells). In the preferred embodiments, the T lineage cells, e.g. autoreactive T lineage cells, are T-cells possessing TCRs directed against healthy tissue including pancreatic β-cells. In further embodiments, the CAR T-cells deplete autoreactive T-cells against healthy host tissues.

In another embodiment, the autoimmune disorder is caused by T lineage cells (e.g. autoreactive T lineage cells) result in inflammation and damage to healthy tissue, such as infiltration of the lamina propria by inflammatory CD4+ T-cell populations in IBD (Crohn’s disease, ulcerative colitis). In further embodiments, the CAR T-cells deplete autoreactive T-cells against healthy host tissues.

In another embodiment, the autoimmune disorder is caused by T lineage cells (e.g. autoreactive T lineage cells) result in inflammation and damage to healthy tissue, such as infiltration by autoreactive T cells in psoriasis. In further embodiments, the CAR T-cells deplete autoreactive T-cells against healthy host tissues.

In another embodiment, graft-versus-host diseases caused by donor T lineage cells result in inflammation and damage to recipient healthy tissue. In further embodiments, the CAR T-cells deplete donor autoreactive T-cells against healthy host tissues.

In preferred embodiments, the autoimmune disorder is caused by both T lineage cells (e.g. autoreactive T lineage cells) and autoreactive antibody produced by B-cells or plasma cells.

In another embodiment, the auto-rejection of organ transplantation caused by autoreactive T-cells or autoreactive antibody produced by B-cells or plasma cells.

In another embodiment, the auto-rejection of organ transplantation caused by both autoreactive T-cells and autoreactive antibody produced by B-cells or plasma cells.

In another embodiment, the present disclosure provides an engineered cell having at least one chimeric antigen receptor polypeptide and an enhancer.

In one embodiment, the present disclosure provides an engineered cell having at least two distinct chimeric antigen receptor polypeptides and an enhancer.

As used herein, an enhancer includes a biological molecule that promotes or enhances the activity of the engineered cell having the chimeric antigen receptor polypeptide. Enhancers include cytokines. In another embodiment, enhancers include IL-2, IL-7, IL-12, IL-15, IL-18, IL-21, PD-1, PD-L1, CSF1R, CTAL-4, TIM-3, and TGFR beta, receptors for the same, TNF-alpha., and functional fragments thereof.

In one embodiment, the present disclosure provides an engineered T-cell, NK-cell having an enhancer selected from the following, IL-15 or IL-15/IL-15sushi or IL-15/IL-15sushi anchor. In this further embodiment, the additional enhancer promotes the CAR T-cell proliferation and persistency.

In another embodiment, the present disclosure provides a method of reducing the number of autoreactive T lineage cells or autoreactive B lineage cells thereof comprising administering a composition comprising (i) an engineered or modified NK or T cell and (ii) an IL-7, IL-15, IL-/IL-15sushi, IL-15/IL-15 sushi anchor, CCL-119 or CCL-21 to said host in need thereof.

In yet another embodiment, a method for ex vivo expansion of NK cells and T cells comprising: 1) isolation of NK or T cells; 2) introduction of at least one CAR; 3) introduction of at least one of enhancers selected from a group of IL-7, IL-15, IL-15sushi, IL-15/IL-15anchor, CCL-19 (CCL19) and CCL-21 (CCL21) and 3) expansion of NK or T cells.

In some embodiments, the autoimmune disorder is selected from T1D, MS, IBD, celiac’s disease, Asthma, systemic lupus erythematosus, IgA nephropathy, IgG4 related disease, membranous nephropathy. Myasthenia gravis, Neuromyelitis optica, Pemphigus vulgaris, anti-PAD4-activating rheumatoid arthritis, Sensitized / preformed antibodies in solid organ transplant, Guillain-Barre Syndrome (Acute inflammatory demyelinating polyneuropathy -AIDP), Chronic inflammatory demyelinating polyneuropathy (CIDP), Immune thrombocytopenic purpura, rheumatoid arthritis, and ANCA-associated vasculitis (AAV). In preferred embodiments, the autoimmune disorder is T-cell mediated autoimmune disease.

In some embodiments, the autoimmune disorder is newly diagnosed (e.g. newly diagnosed T1D, MS, or IBD). In some embodiments, the autoimmune disorder is relapsed or refractory (e.g. relapsed or refractory T1D, MS, IBD).

In another embodiment, the CAR targeting CD7 surface antigen can deplete the autoreactive immune cell expressing the CD7 surface antigen. The CD7+ population will be depleted (approximately >90 T lymphocytes) and an unexpected finding was elucidated in that the CD7- population of T lymphocytes expand to maintain the total T-cell population and prevent infection. Such an occurrence acts as an immune system reset for the T-cell immune system thus treating T-cell mediated autoreactive disease.

In another embodiment, a CD7 monoclonal antibody targeting CD7 surface antigen can deplete the autoreactive immune cell expressing the CD7 surface antigen. The CD7+ population will be depleted (approximately >90 T lymphocytes) and an unexpected finding was elucidated in that the CD7- population of T lymphocytes expand to maintain the total T-cell population and prevent infection. Such an occurrence acts as an immune system reset for the T-cell immune system thus treating T-cell mediated autoimmune disorders

In another embodiment, anti-CD7 CAR targets T lymphocyte lineage cells, particularly T-regulatory cells (Treg cells), which enhance the T-cell expansion. In further embodiments, the CD7CAR can be utilized as a pre-treatment for CAR T-cell cellular therapy. In such embodiments, there exists an unexpected finding, the CD7CAR displays less toxicity and no significant reduction of T-cell number in the treated subject.

In another embodiment, anti-CD7 CAR targets T lymphocyte lineage cells, particularly T-regulatory cells (Treg cells), which enhance the T-cell expansion. In further embodiments, the CD7CAR can be utilized as a pre-treatment for CAR T-cell cellular therapy utilized in combination with pre-treatment pharmaceuticals (cyclophosphamide, fludarabine). In such embodiments, there exists an unexpected finding, the CD7CAR displays less toxicity and no significant reduction of T-cell number in the treated subject.

In another embodiment, the subject can be pre-administered anti-CD7 CAR to deplete T-cell and Treg expressing CD7 surface antigen, followed by administration of a targeted CAR.

In some embodiment, the subject can be pre-administered anti-CD7 CAR to improve CAR T or NK kinetics and initial response and decrease rejection.

In another embodiment, the subject can be pre-administering an anti-CD7 monoclonal antibody can use a combination of cyclophosphamide and fludarabine to improve CAR T and NK cell expansion and decrease rejection.

In another embodiment, the subject can be administering an anti-CD7 monoclonal antibody can use a combination of cyclophosphamide and fludarabine to improve CAR T and NK cell expansion and decrease rejection.

In preferred embodiments, the autoimmune disorder is caused by T lineage cells (e.g. autoreactive T lineage cells). In the preferred embodiments, the T lineage cells, e.g. autoreactive T lineage cells, are T-cells possessing TCRs directed against healthy tissue including pancreatic β-cells. In further embodiments, autoreactive T lineage cells against pancreatic β-cells can be depleted by an anti-CD7 monoclonal antibody.

In another embodiment, the subject can be administered a dual CAR containing CD7CAR and a targeted CAR in a construct.

In another embodiment, the dual CAR may be a tandem CAR, compound CAR, cistronic chimeric antigen receptor CAR and bispecific CAR.

In further embodiments, the anti-CD7 CAR elucidated an unexpected finding whereby greater than 90% of CD7 positive T lineage lymphocytes were depleted and the minority population (2-10%) of T lineage lymphocytes negative for CD7 surface expression expanded along with the CAR T-cell population. The unexpected finding of normal T lineage lymphocyte population numbers following anti-CD7 CAR treatment provided for T-cell immune function for the subject, thus preventing infection.

In certain embodiment, the CD7CAR can be combined with a CAR that requires CAR T-cell expansion. The CD7CAR can be combined with a CAR to select from the following target antigens: at least one of this group, but not limited to, GD2, GD3,, ROR1, PSMA, PSCA (prostate stem cell antigen), MAGE A3, Glycolipid, glypican 3, F77, GD-2, WT1, CEA, HER-2/neu, MAGE-3, MAGE-4, MAGE-5, MAGE-6, alpha-fetoprotein, CA 19-9, CA 72-4, NY-ESO, FAP, ErbB, c-Met, MART-1, MUC1, MUC2, MUC3, MUC4, MUC5, KIF20A, Survivin, AFP-1, gp100, MUC1, PAP-10, PAP-5, TRP2-1, SART-1, VEGFR1, VEGFR2, NEIL3, MPHOSPH1, DEPDC1, FOXM1, CDH3, TTK, TOMM34, URLC10, KOC1, UBE2T, TOPK, ECT2, MESOTHELIN, NKG2D, P1A, GM2, CD30, MMG49 epitope, EGFRvIII, CD33, CD123, CLL-1, immunoglobin kappa and lambda. CD38, CD52, CD47, CD200, CD70, CD19, CD20, CD22, CD38, BCMA, CS1, NKG2D receptor, April receptor, BAFF receptor, TACI, CD3, CD4, CD8, CD5, CD2, GPRC5D (G protein-coupled receptor, class C, group 5, member D) and CD138. The target antigens can also include viral or fungal antigens, such as E6 and E7 from the human papillomavirus (HPV) or EBV (Epstein Barr virus) antigens.

In another embodiment, the present disclosure provides an engineered cell having at least one of recombinant IL-15, IL-15RA, IL-15sushi, IL-15/IL-15RA, IL15-RA/IL-15, IL-15/IL-15sushi, IL15sushi/IL-15, functional fragment thereof, or combination thereof; and at least one distinct CAR polypeptide wherein the antigen recognition domain includes NY-ESO-1, alpha fetoprotein (AFP), glypican-3 (GPC3), BCMA, BAFF-R, BCMA, TACI, LeY, CD5, CD7, CD2, CD3, CD4, CD45, CD13, CD14, CD15, CD19, CD20, CD22, CD33, CD41, CD61, CD64, CD68, CD117, CD123, CD138, CD267, CD269, CD38, Flt3 receptor, ROR1, PSMA, MAGE A3, Glycolipid, F77, GD-2, WT1, CEA, HER-2/neu, MAGE-3, MAGE-4, MAGE-5, MAGE-6, CA 19-9, CA 72-4, NY-ESO, FAP, ErbB, c-Met, MART-1, CD30, EGFRvIII, immunoglobin kappa and lambda, CD38 and CS1. The target antigens can also include viral or fungal antigens, such as E6 and E7 from the human papillomavirus (HPV) or EBV (Epstein Barr virus) antigens. In further embodiment, the antigen recognition polypeptides (scFv) and corresponding polynucleotides for CD2, CD3, CD5, CD7, and CD52 as well as IL-15/IL-15sushi and IL-15sushi are described in more detail publications in PCT Application NO. PCT/US2016/39306 and PCT/US2016/019953, the contents of which are incorporated herein by reference.

In another embodiment, the present disclosure provides an engineered cell to further express other regulatory factors of immune function, such as CCL21, IL-2, IL-4, IL-12, IL-13, IL-17, IL-18, IP-10, CCLA, Flt3L, interferon-gamma, MIP-1 alpha, GM-CSF, M-CSF, TGF-beta, and TNF-alpha.

In another embodiment, the subject can be administered anti-CD7 CAR to deplete T-cell and Treg expressing CD7 surface antigen, followed by administration of a targeted CAR with a targeted antigen at least one of this group, but not limited to, GD2, GD3,, ROR1, PSMA, PSCA (prostate stem cell antigen), MAGE A3, Glycolipid, glypican 3, F77, GD-2, WT1, CEA, HER-2/neu, MAGE-3, MAGE-4, MAGE-5, MAGE-6, alpha-fetoprotein, CA 19-9, CA 72-4, NY-ESO, FAP, ErbB, c-Met, MART-1, MUC1, MUC2, MUC3, MUC4, MUC5, KIF20A, Survivin, AFP-1, gp100, MUC1, PAP-10, PAP-5, TRP2-1, SART-1, VEGFR1, VEGFR2, NEIL3, MPHOSPH1, DEPDC1, FOXM1, CDH3, TTK, TOMM34, URLC10, KOC1, UBE2T, TOPK, ECT2, MESOTHELIN, NKG2D, P1A, GM2, CD30, MMG49 epitope, EGFRvIII, CD33, CD123, CLL-1, immunoglobin kappa and lambda, CD38, CD52, CD47, CD200, CD70, CD19, CD20, CD22, CD38, BCMA, CS1, NKG2D receptor, April receptor, BAFF receptor, TACI, CD3, CD4, CD8, CD5, CD2, and CD138. The target antigens can also include viral or fungal antigens, such as E6 and E7 from the human papillomavirus (HPV) or EBV (Epstein Barr virus) antigens.

Therefore, existing treatments for autoimmune disorders are not always effective in the induction or maintenance of remission and/or may have undesirable side effects. Thus, reasserting the clear and present need for further therapies for the treatment and management of autoimmune disorders. The novel invention is to reset the immune system by depleting disease causing auto- T-cells, B-cells, plasma cells and re-population lymphocyte counts from bone marrow stem cells. One can create the compound chimeric antigen receptor dual CARs where one chimeric antigen receptor (CAR) unit targets for T-cells by selecting one of the target antigens from the following group: CD2, CD3, CD4, CD5, and CD7; and the other CAR unit targets for B-cells by selecting one of the target antigens from the following group: target antigen CD19, CD20, CD22; or plasma cells by selecting one of the target antigens from the following group or target antigen BCMA, CD38, CS1, and CD138.

Multiple extracellular cell markers are now being studied for value as tumor-associated antigens and thus potential targets for CAR T/NK cell therapy. However, expression of these antigens on healthy tissue leading to on-target, off-tumor adverse events remains a major safety concern in addition to off-target toxicities. Furthermore, a major limitation of CAR T/NK cell therapy is in the possibility of selecting for antigen escape variants when targeting molecules non-essential to tumorigenesis. Thus, malignant cells that persist with little or no expression of the target antigens may evade CAR T/NK cells, despite their high-affinity action.

In accordance with the present invention, natural killer (NK) cells represent alternative cytotoxic effectors for CAR driven killing. Unlike T-cells, NK cells do not need pre-activation and constitutively exhibit cytolytic functions. Further expression of cCARs in NK cells allow NK cells to effectively kill cancers, particularly cancer cells that are resistant to NK cell treatment.

Further, NK cells are known to mediate anti-cancer effects without the risk of inducing graft-versus-host disease (GvHD).

Studies have shown an aberrant overexpression of CD123 on CD34+ CD38- AML cells, while the normal bone marrow counterpart CD34+ CD38- does not express CD123(Jordan, Upchurch et al. 2000). This population of CD123+, CD34+CD38- has been considered as LSCs as these cells are able to initiate and maintain the leukemic process into immunodeficient mice.

The number of CD34+ /CD38- /CD123+ LSCs can be used to predict the clinical outcome for AML patients. The CD34+ /CD38- /CD123+ cells, greater than 15% in AML patients, are associated with a lack of complete remission and unfavorable cytogenetic profiles. In addition, the presence of more than 1% of CD34+ /CD38- /CD123+ cells could also have a negative impact on disease-free survival and overall survival.

At the present, therapies for MDS and AML have focused on the leukemic blast cells because they are very abundant and clearly represent the most immediate problem for patients. Importantly, leukemic stem cells (LSCs) are quite different from most of the other leukemia cells (“blast” cells), and they constitute a rare subpopulation. While killing blast cells can provide short-term relief, LSCs, if not destroyed, will always re-grow, causing the patient to relapse. It is imperative that LSCs be destroyed in order to achieve durable cures for MDS disease. Unfortunately, standard drug regimens are not effective against MDS or AML LSCs. Therefore, it is critical to develop of new therapies that can specifically target both the leukemic stem cell population and the bulky leukemic population. The compound CAR disclosed in the present invention target both of these populations and is embodied herein.

In accordance to the present invention, it was surprisingly found that NK cells provide an off-the-shelf product that may be used as an allogeneic product for treatment. Thus, according to the present invention, cCAR cell therapy needs to be performed on a patient-specific basis as required by the current state of art. The applicants of the present invention have discovered a novel immunotherapy, where the patient’s lymphocytes or tumor infiltrated lymphocytes need not be isolated for an effective CAR cell based therapy.

Allogeneic or autologous NK cells are expected to induce a rapid immune response but disappear relatively rapidly from the circulation due to their limited lifespan. Thus, applicants surprisingly discovered that there is reduced concern of persisting side effects using cCAR cell based therapy.

According to one aspect of the present invention, NK cells can be expanded and transfected with cCAR in accordance to the present invention. NK cells can be derived from cord blood, peripheral blood, iPS cells and embryonic stem cells. According to one aspect of the present invention, NK-92 cells may be expanded and transfected with cCAR. NK-92 is a continuously growing cell line that has features and characteristics of natural killer (NK) cells. NK-92 cell line is IL-2 dependent and has been proven to be safe and feasible. cCAR expressing NK-92 cells can be expanded in the serum free-medium with or without co-culturing with feeder cells. A pure population of NK-92 carrying the cCAR of interest may be obtained by sorting.

Identification of appropriate surface target antigens is a prerequisite for developing CAR T/NK cells in adaptive immune therapy.

In one aspect of the present invention, CD123 antigen is one of the targets for cCAR therapy. CD123, the alpha chain of the interleukin 3 receptor, is overexpressed on a variety of hematologic malignancies, including acute myeloid leukemia (AML), B-cell acute lymphoblastic leukemia (B-ALL), hairy cell leukemia, and blastic plasmocytoid dendritic neoplasms. CD123 is absent or minimally expressed on normal hematopoietic stem cells. More importantly, CD123 is expressed on a subset of leukemic cells related to leukemic stem cells (LSCs), the ablation of which is essential in preventing disease refractoriness and relapse.

In one aspect of the present invention, CD 33 antigen is one of the targets for cCAR therapy. CD33 is a transmembrane receptor expressed on 90% of malignant cells in acute myeloid leukemia. Thus, according to the present invention, CD123 and CD33 target antigens are particularly attractive from a safety standpoint.

In accordance with the present invention, the compound CD33CD123 CARs may be highly effective for therapeutic treatment of chronic myeloid leukemia (CML) population. In chronic myeloid leukemia (CML), there is a rare subset of cells that are CD34+CD38-. This population is considered as comprised of LSCs. Increased number of LSCs is associated with the progression of the disease. A small-molecule Bcr-Abl tyrosine kinase inhibitor (TKI) is shown to significantly improve the overall survival in CP-CML patients. However, LSCs are thought to be resistant to TKI therapy. A novel therapy targeting CML resistant LSCs is urgently needed for treatment of CML and the novel therapy is embodied in the compound CD33CD123 CAR disclosed in the present invention. CD123 expression is high in the CD34+CD38-population. In accordance with the present invention, the compound CD33CD123 CARs is highly effective for therapeutic treatment of this population.

In one embodiment of the present invention, leukemic cells expressing both CD123 and CD33 in the cCAR is used as a therapeutic treatment. CD33 is expressed on cells of myeloid lineage, myeloid leukemic blasts, and mature monocytes but not normal pluripotent hematopoietic stem cells (Griffin, Linch et al. 1984). CD33 is widely expressed in leukemic cells in CML, myeloproliferative neoplasms, and MDS.

As a significant number of patient with acute myeloid leukemia (AML) are refractory to standard chemotherapy regimens or experience disease relapse following treatment (Burnett 2012), the development of CAR T cell immunotherapy for AML has the potential to address a great clinical need. In the majority of these patients, leukemic cells express both CD123 and CD33, giving broad clinical applicability to the compound CD33CD123 CAR disclosed herein. Thus, the present invention discloses a novel multiple cCAR T/NK cell construct comprising multiple CAR targeting multiple leukemia-associated antigens, thereby offsetting antigen escape mechanism, targeting leukemia cells, including leukemic stem cells, by synergistic effects of co-stimulatory domain activation and thereby providing a more potent, safe and effective therapy.

The present invention further discloses compound CAR construct, with enhanced potency of anti-tumor activity against cells co-expressing target antigens, and yet retains sensitivity to tumor cells only expressing one antigen. In addition, each CAR of the compound CAR includes one or two co-stimulatory domains and potent killing capability in presence of the specific target.

In pre-clinical studies on dual specificity, trans-signaling CARs targeting solid tumors including breast cancer and epithelial ovarian cancer, a CD3ζ intracellular signaling domain is separated from co-stimulatory domains from second generation of CARs. In other words, one CAR contains the first generation of CAR without any co-stimulatory domain, and another lacks a CD3 zeta intracellular domain. Therefore, the presence of both target antigens is required for T cell activation and potent killing. Thus, they were proposed as a way to decrease off-tumor toxicity caused by healthy tissue expression of one of the two target antigens, increasing target specificity, but at the expense of sensitivity. In one embodiment, the compound CAR is a compound CD123CD19 CAR. It has been shown that more than 90% of B-ALLs express CD123 in a subset of population. Like AML and MDS, it has been considered that a rare LSC population exists in B-ALL. Therefore, targeting both leukemic stem cell and bulky leukemic populations in accordance to the present invention, can be applied to B-ALLs. CD123 and CD19 surface antigens expressed in the B-ALLs may be targets as CD19 is widely expressed in different stages of B-cell lymphoid populations, in accordance with the present invention.

Multiple myeloma (MM) is the second most common hematologic malignancy in the US and is derived from clonal plasma cells accumulated in the bone marrow or extramedullary sites. MM is an incurable disease with a median survival of approximately 4.5 years (Kumar, Rajkumar et al. 2008). Anti-Myeloma CARs in Pre-clinical Development have been developed and CAR targets include CD38, CS1, B cell maturation Antigen (BCMA) and CD38. However, heterogeneity of surface antigen expression commonly occurs in malignant plasma cells (Ruiz-Arguelles and San Miguel 1994), which makes it a difficult target for CARs. Malignant plasma cells also express low levels of CD19. Previously it has been shown that myeloma stem cells also express some B-cell markers including CD19. Targeting this population could be effective in the treatment of myeloma in conjunction with standard and other myeloma CAR therapies.

Multiple myeloma (MM) is a haematological malignancy with a clonal expansion of plasma cells. Despite important advances in the treatment, myeloma remains an incurable disease; thus novel therapeutic approaches are urgently needed.

CS1 (also called as CD319 or SLAMF7) is a protein encoded by the SLAMF7 gene. The surface antigen CS1 is a robust marker for normal plasma cells and myeloma cells (malignant plasma cells).

Tumour necrosis factor receptor superfamily, member 17 (TNFRSF17), also referred to as B-cell maturation antigen (BCMA) or CD269 is almost exclusively expressed at the terminal stages of plasma cells and malignant plasma cells. Its expression is absent other tissues, indicating the potential as a target for CAR T or NK cells.

Malignant plasma cells display variable degrees of antigenic heterogeneity for CD269 and CS1. A single CAR unit product targeting either CD269 or CS1 could target the majority of the cells in a bulk tumor resulting in an initial robust anti-tumor response. Subsequently residual rare non-targeted cells are expanded and cause a disease relapse. While multiple myeloma is particularly heterogeneous, this phenomena could certainty apply to other leukemias or tumors. A recent clinical trial at NIH using BCMA CAR T cells showed a promising result with a complete response in some patients with multiple myeloma. However, these patients relapsed after 17 weeks, which may be due to the antigen escape. The antigen escape is also seen in CD19 CAR and NY-ESO1 CAR T cell treatments. Thus, there is an urgent need for more effective CAR T cell treatment in order to prevent the relapse.

In one aspect of the present invention, BCMA and CS1 are the targets for BCMACS1 CAR therapy.

In some embodiments, a compound CAR targets cells expressing BCMA or CS1 antigens or both. The targeted cells may be cancer cells, such as, without limiting, lymphomas, or leukemias or plasma cell neoplasms. In further embodiments, plasma cell neoplasms is selected from plasma cell leukemia, multiple myeloma, plasmacytoma, heavy chain diseases, amyloidosis, waldestrom’s macroglobulinema, heavy chain diseases, solitary bone plamacytoma, monoclonal gammopathy of undetermined significance (MGUS) and smoldering multiple myeloma.

BAFF (B-cell-activation factor) and APRIL (a proliferation-induced ligand) are two TNF homologs that bind specifically TACI (also called as TNFRSF1 3B or CD267) and BCMA with high affinity. BAFF (also known as BLyS) binds BAFF-R and functionally involves in the enhancement of survival and proliferation of later stage of B cells. BAFF has been shown to involve some autoimmune disorders. APRIL plays an important role in the enhancement of antibody class switching. Both BAFF and APRIL have been implicated as growth and survival factors for malignant plasma cells.

Ligand-receptor interactions in the malignant plasma cells are described in FIG. 45.

In some embodiments, a compound CAR targets cells expressing TACI or CS1 antigens or both. In another embodiment, a compound CAR targets cells expressing TACI or CS1 antigens or both. The targeted cells may be cancer cells, such as, without limiting, lymphomas, or leukemias or plasma cell neoplasms. In further embodiments, plasma cell neoplasms is selected from plasma cell leukemia, multiple myeloma, plasmacytoma, heavy chain diseases, amyloidosis, waldestrom’s macroglobulinema, heavy chain diseases, solitary bone plamacytoma, monoclonal gammopathy of undetermined significance (MGUS) and smoldering multiple myeloma. The target cells may also be one or two or multiple different cell types of B cells, immature B cells, naïve B cells, centroblasts, centrocytes, memory B cells, plasmablasts, long lived plasma cells, plasma cells. These cells involve autoimmune diseases include systemic scleroderma, multiple sclerosis, psoriasis, dermatitis, inflammatory bowel diseases (such as Crohn’s disease and ulcerative colitis), systemic lupus erythematosus, vasculitis, rheumatoid arthritis, Sjorgen’s syndrome, polymyositis, granulomatosis and vasculitis, Addison’s disease, antigen-antibody complex mediated diseases, and anti-glomerular basement membrane disease.

In some embodiments, a compound CAR targets cells expressing BAFF-R or CS1 antigens or both. In another embodiment, a compound CAR targets cells expressing BAFF-R or CS1 antigens or both. The targeted cells may be cancer cells, such as, without limiting, lymphomas,or leukemias or plasma cell neoplasms. In further embodiments, plasma cell neoplasms is selected from plasma cell leukemia, multiple myeloma, plasmacytoma, heavy chain diseases, amyloidosis, waldestrom’s macroglobulinema, heavy chain diseases, solitary bone plamacytoma, monoclonal gammopathy of undetermined significance (MGUS) and smoldering multiple myeloma.

In some embodiments, a compound CAR (cCAR) targets cells expressing one or two or all of BAFF-R, BCMA, TACI and CS1 antigens.

In some embodiments, a unit of CAR in a cCAR can comprise: 1)a scFv against either BAFF-R, BCMA, TACI and CS1; 2) a hinge region; 3)co-stimulatory domain (s) and intracellular signaling domain.

In some embodiments, a unit of CAR in a cCAR can comprise: 1) BCMA or TACI or BAFF-R binding domain, or APRIL binding domain; 2) a hinge region; 3) co-stimulatory domain (s) and intracellular signaling domain.

In a further embodiment, BCMA or TAC1 or BAFF-R binding domain can be a part of or entire APRIL and BAFF molecules.

In some embodiments, a unit of CAR in a cCAR can comprise: 1) a scFv against BCMA or CS1; 2) a hinge region; 3)co-stimulatory domain (s) and intracellular signaling domain.

In further embodiments, cCAR can comprise one or two or multiple units of CAR. Each unit CAR could bear same or different hinge region and co-stimulatory domain.

In further embodiments, the target antigens can include at least one of this group, but not limited to, ROR1, PSMA, MAGE A3, Glycolipid, glypican 3, F77, GD-2, WT1, CEA, HER-2/neu, MAGE-3, MAGE-4, MAGE-5, MAGE- 6, alpha-fetoprotein, CA 19-9, CA 72-4, NY-ESO, FAP, ErbB, c-Met, MART-1, CD30, EGFRvIII, immunoglobin kappa and lambda, CD38, CD52, CD3, CD4, CD8, CD5, CD7, CD2, and CD138. The target antigens can also include viral or fungal antigens, such as E6 and E7 from the human papillomavirus (HPV) or EBV (Epstein Barr virus) antigens.

In some embodiments, a cCAR targets a cell expressing either CD19 or CD20 antigens or both of them. In another embedment, a cCAR targets a cell expressing either CD19 or CD22 antigens or both of them. The targeting cells are cancer cells such as B-cell lymphomas or leukemias.

Acute graft-versus-host disease (GVHD) remains the most important cause of morbidity and mortality after allogeneic hematopoietic stem cell transplantation. In the effector phase of GVHD, T cell receptor (TCR), a heterodimer of alpha and beta chains, is expressed on the surface of T cells, TCR recognizes some antigens on the HLA molecule on host cells, enhances T cell proliferation, and releases cytotoxic agents that cause the damage on host cells. TCR gene inactivation is efficient at preventing potential graft-versus-host reaction. The inactivation of TCRs can result in the prevention of the TCR recognition of alloantigen and thus GVHD. The role of CD45 on NK cells is quite different from that of T cells. NK cells from CD45-difficient mice have normal cytotoxic activity against the prototypic tumor cell line, Yac-1. In addition, CD45-deficient NK cells proliferate normally and respond to IL15 and IL-21. Therefore, CD45 disruption or deletion would not affect the NK cell killing and proliferation. The present disclosure includes methods of permanent deletion of CD45 in a T or NK cell with subsequent stable introduction of CD45-specific CARs. As a result, the engineered T cells display the desired properties of redirected specificity for CD45 without causing self-killing and response to presentation of antigen. In a further embodiment, the engineered T cells may have efficacy as an off-the-shelf therapy for treating malignancies or other diseases. The present disclosure relates to a method where T-cells are engineered to allow proliferation when TCR signaling is reduced or lost through the inactivation or deletion of endogenous CD45. The reduction or loss of TCR signaling could result in the prevention of GVHD. In a further embodiment, T cells reducing or losing the TCR signaling by the inactivation of CD45 could be used as an “off the shelf “ therapeutic product.

The present disclosure includes methods of modified T or NK cells, which comprises: (a) modifying T or NK cells by inactivating CD45; (b) expanding these modified cells; (c) sorting modified T or NK cells, which do not express CD45; (d) introducing CD45CAR. In embodiments, the CD45CAR gene encodes a chimeric antigen receptor (CAR), wherein the CAR comprises at least one of an antigen recognition domain, a hinge region, a transmembrane domain, and T cell activation domains, and the antigen recognition domain is redirected against CD45 surface antigen present on a cell. The antigen recognition domain includes a monoclonal antibody or a polyclonal antibody directed against CD45 antigen. The antigen recognition domain includes the binding portion or a variable region of a monoclonal or a polyclonal antibody.

In some embodiments, the modified T cells are obtained from allogeneic donors and used as an ‘off-the-shelf product”.

Targeting CD45 using CAR T or NK cells may cause self-killing as T and NK cells express this surface antigen. To overcome this drawback, the inventor proposes to inactivate CD45 gene using engineered CRISPR/Cas9 system, zinc finger nuclease (ZFNs) and TALE nucleases (TALENs) and meganucleases. The loss of CD45 in T or NK cells is further transduced with CARs targeting neoplasms expressing CD45.

The disclosure includes methods for eliminating or reducing abnormal or malignant cells in bone marrow, blood and organs. In some embodiments, malignant cells expressing CD45 are present in patients with acute leukemia,, chronic leukemia, B and T cell lymphomas, myeloid leukemia, Acute lymphoblastic lymphoma or leukemia, primary effusion lymphoma, Reticulohistiocytoma, transient myeloproliferative disorder of Down’s syndrome, lymphocyte predominant Hodgkin’s lymphoma, myeloid leukemia or sarcoma, dendrocytoma, histiocytic sarcoma, Giant cell tumor of tendon sheath, interdigitating dendritic cell sarcoma, post-transplant lymphoproliferative disorders, etc.

In some embodiments, CD45CAR cells can be used to make space in the bone marrow for bone marrow stem cell transplant by removing hematopoietic cells, at the same time removing leukemic/lymphoma cells or immunologic cells capable of graft rejection. In a further embodiment, CD45CAR cells may be used for pre-treatment of patients before their undergoing a bone marrow transplant to receive stem cells. In a further embodiment, CD45CAR can be used as myeloblative conditioning regimens for hematopoietic stem cell transplantation.

In some embodiment, CD45CAR cells are utilized for treating or preventing a residual disease after stem cell transplant and/or chemotherapy.

In some embodiments, the CD45CAR is part of an expressing gene or a cassette. In a preferred embodiment, the expressing gene or the cassette may include an accessory gene or a tag or a part thereof, in addition to the CD45CAR. The accessory gene may be an inducible suicide gene or a part thereof, including, but not limited to, caspase 9 gene, thymidine kinase, cytosine deaminase (CD) or cytochrome P450. The “suicide gene” ablation approach improves safety of the gene therapy and kills cells only when activated by a specific compound or a molecule. In some embodiments, the suicide gene is inducible and is activated using a specific chemical inducer of dimerization (CID).

In some embodiments, safety switch can include the accessory tags are a c-myc tag, CD20, CD52 (Campath), truncated EGFR gene (EGFRt) or a part or a combination thereof. The accessory tag may be used as a nonimmunogenic selection tool or for tracking markers. In some embodiments, safety switch can include a 24-residue peptide that corresponds to residues 254-277 of the RSV F glycoprotein A2 strain (NSELLSLINDMPITNDQKKLMSNN). In some embodiments, safety switch can include the amino acid sequence of TNF α bound by monoclonal anti-TNF α drugs.

Administration of any of the engineered cells described herein may be supplemented with the co-administration of a CAR enhancing agent. Examples of CAR enhancing agents include immunomodulatory drugs that enhance CAR activities, such as, but not limited to agents that target immune-checkpoint pathways, inhibitors of colony stimulating factor-1 receptor (CSF1R) for better therapeutic outcomes. Agents that target immune-checkpoint pathways include small molecules, proteins, or antibodies that bind inhibitory immune receptors CTLA-4, PD-1, and PD-L1, and result in CTLA-4 and PD-1/PD-L1 blockades. As used herein, enhancing agent includes enhancer as described above.

As used herein, “patient” includes mammals. The mammal referred to herein can be any mammal. As used herein, the term “mammal” refers to any mammal, including, but not limited to, mammals of the order Rodentia, such as mice and hamsters, and mammals of the order Logomorpha, such as rabbits. The mammals may be from the order Carnivora, including Felines (cats) and Canines (dogs). The mammals may be from the order Artiodactyla, including Bovines (cows) and Swines (pigs) or of the order Perssodactyla, including Equines (horses). The mammals may be of the order Primates, Ceboids, or Simoids (monkeys) or of the order Anthropoids (humans and apes). Preferably, the mammal is a human.

A patient in need thereof includes patients suffering from a disease that would benefit from the claimed methods of treatment or a patient at risk for developing a disease that would benefit from the claimed methods of treatment includes subject.

For example, high risk for developing type 1 diabetes is defined as: (i) Having at least one diabetes related autoantibodies confirmed to be present on at least one occasion. This includes anti-GAD65, anti-ICA512, anti-insulin (MIAA), and ICA (islet cell antibody); (ii) Having an abnormal glucose tolerance by OGTT (oral glucose tolerance test); (iii) Having fasting plasma glucose ≥ 110 mg/dL and < 126 mg/dl; (iv) and/or 2 hour plasma glucose ≥ 140 mg/dL, and < 200 mg/dl; (v) and/or 30, 60, or 90 minute value on OGTT≥200 mg/dl.

To date, more than 100 identified autoimmune disorders exist, however, treatment is suboptimal and none of them are curative. The standard of care treatment for B-cell and T-cell mediated autoimmune disorders acts to delay the progression of the disease and/or may be supportive care to suppress symptoms, as well as reduce the frequency of disease relapse. The innovation disclosed by the inventor is a novel approach to reset the immune system and thus provide a curative intervention against the autoreactive B-cells, T-cells and Plasma cells. This technology of disclosure acts to deplete autoreactive B-cells, T-cells and plasma cells. The innovation disclosed by the inventor is to reset the patient’s immune system and halt the progression of disease as well as minimize cells responsible for the disease and minimize relapse.

The disclosure is a novel approach to reset the immune system. In a further embodiment the disclosure resets the immune system via a CAR. therapy, whereby this approach treats patients with a high risk for developing an autoimmune disorder and subsequent organ damage which may manifest to be substantial.

In certain embodiments, the patient is a human 0 to 6 months old, 6 to 12 months old, 1 to 5 years old, 5 to 10 years old, 5 to 12 years old, 10 to 15 years old, 15 to 20 years old, 13 to 19 years old, 20 to 25 years old, 25 to 30 years old, 20 to 65 years old, 30 to 35 years old, 35 to 40 years old, 40 to 45 years old, 45 to 50 years old, 50 to 55 years old, 55 to 60 years old, 60 to 65 years old, 65 to 70 years old, 70 to 75 years old, 75 to 80 years old, 80 to 85 years old, 85 to 90 years old, 90 to 95 years old or 95 to 100 years old.

The terms “effective amount” and “therapeutically effective amount” of an engineered cell as used herein mean a sufficient amount of the engineered cell to provide the desired therapeutic or physiological or effect or outcome. Such, an effect or outcome includes reduction or amelioration of the symptoms of cellular disease. Undesirable effects, e.g. side effects, are sometimes manifested along with the desired therapeutic effect; hence, a practitioner balances the potential benefits against the potential risks in determining what an appropriate “effective amount” is. The exact amount required will vary from patient to patient, depending on the species, age and general condition of the patient, mode of administration and the like. Thus, it may not be possible to specify an exact “effective amount”. However, an appropriate “effective amount” in any individual case may be determined by one of ordinary skill in the art using only routine experimentation. Generally, the engineered cell or engineered cells is/are given in an amount and under conditions sufficient to reduce proliferation of target cells.

Following administration of the delivery system for treating, inhibiting, or preventing a cancer, the efficacy of the therapeutic engineered cell can be assessed in various ways well known to the skilled practitioner. For instance, one of ordinary skill in the art will understand that a therapeutic engineered cell delivered in conjunction with the chemo-adjuvant is efficacious in treating or inhibiting a cancer in a patient by observing that the therapeutic engineered cell reduces the cancer cell load or prevents a further increase in cancer cell load. Cancer cell loads can be measured by methods that are known in the art, for example, using polymerase chain reaction assays to detect the presence of certain cancer cell nucleic acids or identification of certain cancer cell markers in the blood using, for example, an antibody assay to detect the presence of the markers in a sample (e.g., but not limited to, blood) from a subject or patient, or by measuring the level of circulating cancer cell antibody levels in the patient.

Throughout this specification, quantities are defined by ranges, and by lower and upper boundaries of ranges. Each lower boundary can be combined with each upper boundary to define a range. The lower and upper boundaries should each be taken as a separate element.

Reference throughout this specification to “one embodiment.” “an embodiment,” “one example,” or “an example” means that a particular feature, structure or characteristic described in connection with the embodiment or example is included in at least one embodiment of the present embodiments. Thus, appearances of the phrases “in one embodiment,” “in an embodiment,” “one example,” or “an example” in various places throughout this specification are not necessarily all referring to the same embodiment or example. Furthermore, the particular features, structures or characteristics may be combined in any suitable combinations and/or subcombinations in one or more embodiments or examples. In addition, it is appreciated that the figures provided herewith are for explanation purposes to persons ordinarily skilled in the art and that the drawings are not necessarily drawn to scale.

As used herein, the terms “comprises.” “comprising,” “includes,” “including,” “has,” “having,” or any other variation thereof, are intended to cover a non-exclusive inclusion. For example, a process, article, or apparatus that comprises a list of elements is not necessarily limited to only those elements but may include other elements not expressly listed or inherent to such process, article, or apparatus.

Further, unless expressly stated to the contrary, “or” refers to an inclusive “or” and not to an exclusive “or”. For example, a condition A or B is satisfied by any one of the following: A is true (or present) and B is false (or not present), A is false (or not present) and B is true (or present), and both A and B are true (or present).

Additionally, any examples or illustrations given herein are not to be regarded in any way as restrictions on, limits to, or express definitions of any term or terms with which they are utilized. Instead, these examples or illustrations are to be regarded as being described with respect to one particular embodiment and as being illustrative only. Those of ordinary skill in the art will appreciate that any term or terms with which these examples or illustrations are utilized will encompass other embodiments which may or may not be given therewith or elsewhere in the specification and all such embodiments are intended to be included within the scope of that term or terms. Language designating such nonlimiting examples and illustrations includes, but is not limited to: “for example,” “for instance,” “e.g.,” and “in one embodiment.”

In this specification, groups of various parameters containing multiple members are described. Within a group of parameters, each member may be combined with any one or more of the other members to make additional sub-groups. For example, if the members of a group are a, b, c, d, and e, additional sub-groups specifically contemplated include any one, two, three, or four of the members, e.g., a and c; a, d, and e; b, c, d, and e; etc.

As used herein, a XXXX antigen recognition domain is a polypeptide that is selective for XXXX. Therefore, XXXX is the target. For example, a CD38 antigen recognition domain is a polypeptide that is specific for CD38.

As used herein, CDXCAR refers to a chimeric antigen receptor having a CDX antigen recognition domain.

The present disclosure may be better understood with reference to the examples, set forth below. The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure.

### EXAMPLES

### Generation of Compound CAR (cCAR)

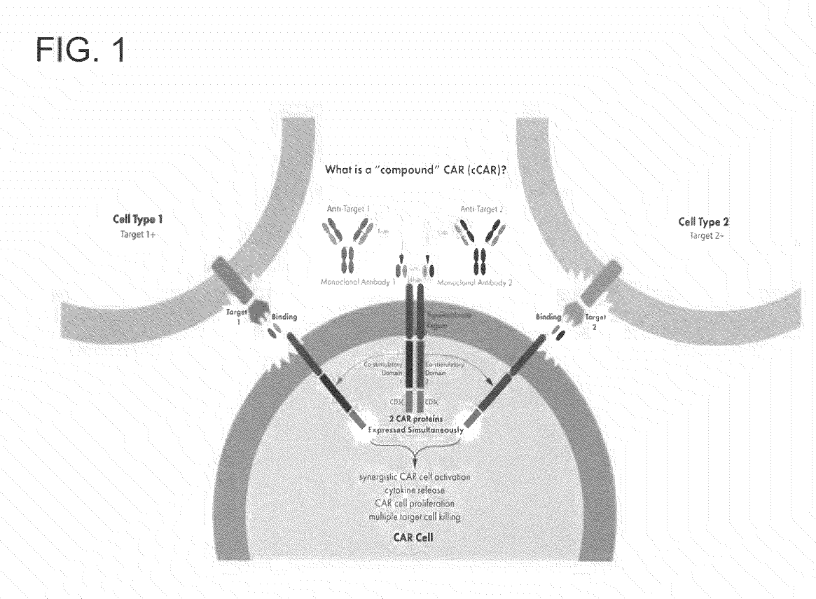

The construction of the CD33CD123 cCAR follows the schematic in FIG. 1A. It includes SFFV (spleen focus-forming virus) promoter that drives the expression of the functional compound CAR (cCAR) bearing two different units of CARs. The antigen receptor head, a scFv (single-chain variable fragment) nucleotide sequence of the anti-CD33 and anti-CD123. A P2A peptide derived from picornavirus is utilized due to the highly efficient mechanism of its self-cleaving dynamics for bicistronic genetic constructs. The self-cleaving P2A peptide serves to link the two independent units of CARs, CD33CAR, and CD123CAR together during expression. The advantages of this approach over an internal ribosomal entry site (IRES), which is commonly used in the literature, include its small size and high cleavage efficiency between two unit proteins upstream and downstream of the 2A peptide. In addition, the use of self-cleaving P2A peptide can avoid a problem of differences in expression levels between gene before and after IRES when IRES is applied.

The modular unit, CD33CAR includes the CD33 scFv domain, a CD8a hinge region, a CD8a transmembrane domain, 4-BB co-stimulatory domain and an intracellular domain of CD3 zeta chain. The second modular CAR, CD123CAR bears the same hinge, transmembrane and intracellular signaling domains as CD33CAR but different scFv, and co-stimulatory domains. The CD33 CAR recognizes its corresponding antigen and the CD123 CAR binds to its corresponding antigen. The hinge region was designed such that sequences where disulfide interactions are avoided. Different co-stimulatory domains, 4-BB and CD28 were used. The CD33CD123 compound CAR was subcloned into a lentiviral plasmid.

### Generation of a High-Efficiency Compound CAR (cCAR)

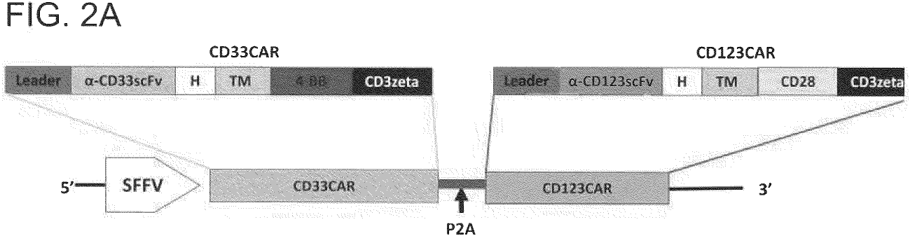

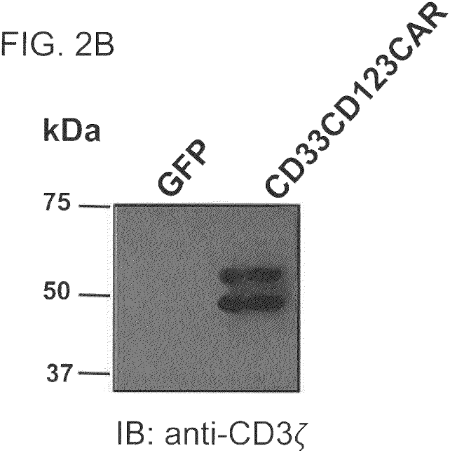

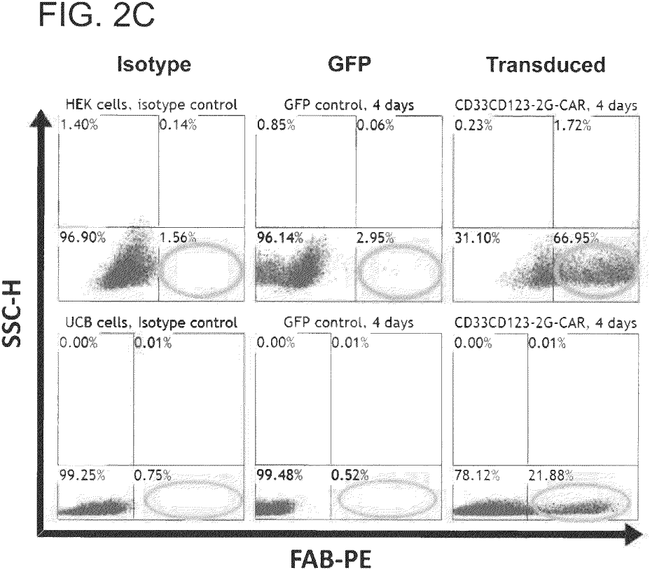

Compound CAR lentivirus was generated by transfection of HEK-293 FT cells with Lipofectamine 2000 according to manufacturer’s directions, except with 2x the vector DNA due to a large size of insert, in order to increase titer as shown in FIG. 2. After about 12-16 hours incubation, media containing Lipofectamine was removed and replaced with DMEM containing 10% FBS, 20 mM HEPES, 1 mM sodium pyruvate and 1 mM sodium butyrate. After about 24 hours, thes supernatant was harvested and refrigerated, and replaced with fresh media. After about another 24 hours, this was collected and combined with the previous supernatant, and filtered through a 0.45 µM filter disc. Supernatant was split into aliquots, flash frozen with liquid nitrogen and stored at -80° C. HEK-293 FT cells were harvested, stored frozen, and lysed for subsequent electrophoresis and Western blotting.

PB (peripheral blood) or CB (human umbilical cord blood) buffy coat cells were activated 2 days with anti-CD3 antibody and IL-2. cCAR lentiviral supernatant was spinoculated onto retronectin-coated multiwell plates. Activated T cells were transduced in multiple wells with lentiviral supernatant at a low concentration of about 0.3 × 10⁶ cells/mL to increase transduction efficiency (FIG. 2).

Following the first overnight transduction, cells were added directly to a second virus-coated plate for a second transduction without washing, unless the cells did not look healthy. Following the second overnight transduction, cells were washed, combined and incubated in tissue culture treated plates. CAR T cells were allowed to expand for up to about 5 days prior to co-culture killing assays. After about 3 days of incubation, cells were incubated with goat anti-mouse F(Ab′)2 or goat IgG (isotype) antibodies conjugated with biotin, washed and followed by incubation with streptavidin-PE and conjugated anti-human CD3. After washing and suspension in 2% formalin, cells were then analyzed by flow cytometry to determine percent transduction efficiency.

### Characterization of the CD33CD123 cCAR

Transfected CD33CD123 cCAR HEK293T cells were subjected to Western blot analysis in order to confirm the compound construct. Immunoblot with an anti-CD3ζ monoclonal antibody showed bands of predicted size for the compoundCAR CD3ζ fusion protein (FIG. 1B). Importantly, two distinct bands of similar intensity were observed on the blot signaling the successful high cleavage action of the P2A peptide as expected. No CD3ζ expression was seen for the GFP control vector as expected. The surface expression of scFv was also tested on HEK 293 cells (FIG. 1C) and primary T cells (FIG. 1C).

The compound CD33CD23CAR lentivirus was tested for transduction efficiency in the HEK293 cell line and analyzed by flow cytometry (Beckman Coulter) (FIG. 1C). Flow cytometry showed that about 67% of HEK cells expressed CD33CD123 CARs. Human peripheral blood (PB) is often used for autologous T cell therapy. Human PB buffy coat cells were activated with anti-CD3 antibody and IL-2, and transduced with either CD4CAR or control (GFP) lentiviruses. After transduction, flow cytometry analysis showed that about 22% of T-cells expressed the CD33CD123CAR (FIG. 1C).

### RESULTS

### CD33CD123 cCAR T-Cells Derived From Umbilical Cord Blood (UCB) and Peripheral Blood (PB) Specifically Kill CD33-Expressing Tumor Cells

CD33CD123 cCAR T cells or GFP T cells (control) were incubated with target cells at ratios ranging from 0.5:1 from 50:1, preferably, at about 2:1, 5:1, 10:1, 20:1, 50:1, at about 100,000, 200,000, 500,000, about 1 million, or 2 million effector cells to about 50,000, 100,000, 200,000 target cells, respectively) in about 1-2 mL T cell culture media, without IL-2 for about 24 h. Target cells were leukemic cell lines and leukemia cells from a patient with leukemia. After about 24 hours of co-culture, cells were stained with mouse anti-human CD33, CD123, CD34 and CD3 antibodies.

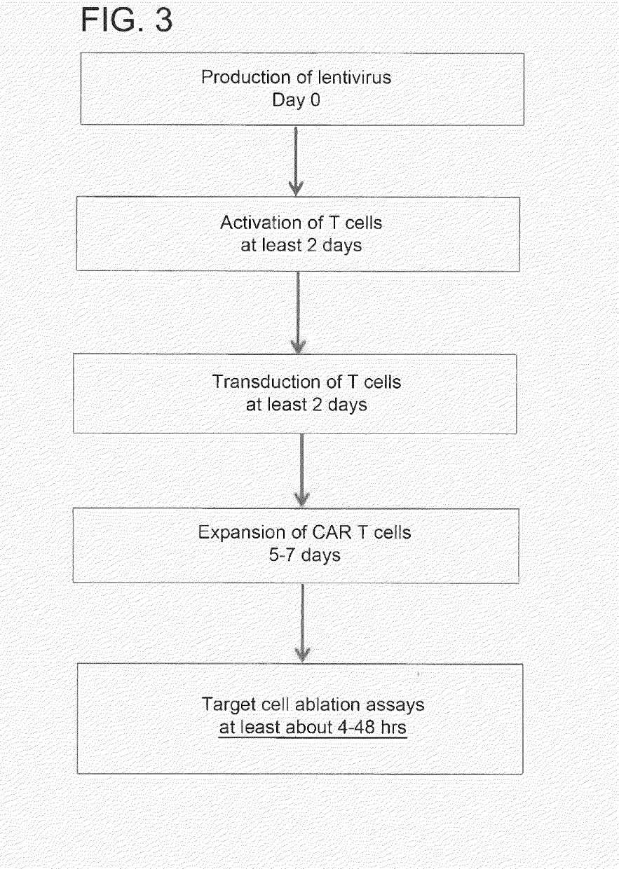

CD33CD123 cCAR T cells expressing the CD33CAR and CD123 CAR were generated and tested for anti-leukemic functions using the HL60 and KG-1a cell lines. The HL60 cell line is a promyelocytic leukemia cell line highly enriched for CD33. About 100% of its cell population is CD33+ with a small subset (<10%) of it being dim CD123+. In culture, this cell line was tested to determine the effectiveness of the CD33CD123 CAR with an emphasis on targeting CD33-expressing leukemic cells. Additionally, due to the strong expression of CD33 in HL60, it is CD33CD123 cCAR action may be profound. Indeed, during 24 h co-culture conditions with various ratios of effector to target cells, the CD33CD123 cCAR exhibited significant leukemic cell killing properties (FIG. 3). CB-derived CD33CD123 CAR T-cells were first tested for their ability to kill HL60 cells. At about 24h incubation and low effector:target (E:T) ratios ranging from about 0.5:1 to 50:1, preferably, 1:1 to about 5:1, more preferably about 2:1 to 4:1, CD33CD123 CAR cells eliminated about 55% of the CD33 expressing HL60 cells when compared to GFP control. At a ratio of about 5:1, the killing action rose to about 82%.

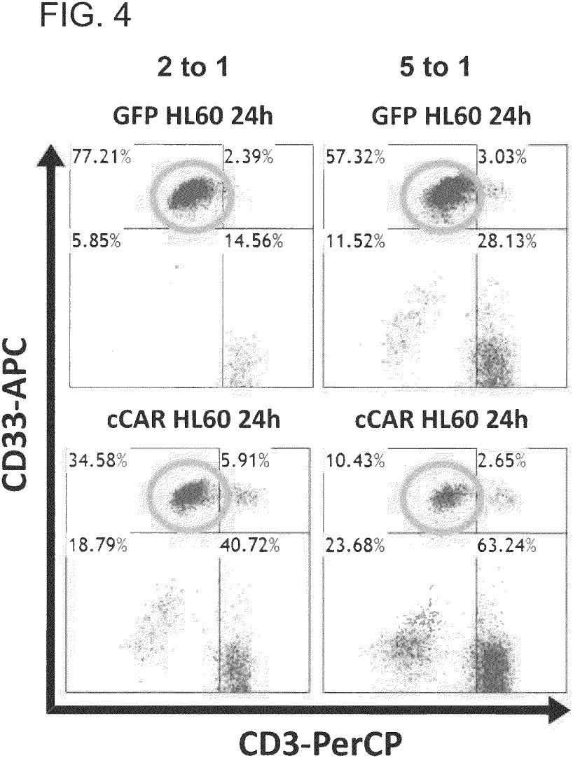

CD33CD123 CAR derived from peripheral blood mononuclear cells (PBMCs) were co-cultured with the myelogenous leukemia cell line KG1a, which also expresses about 100% CD33 at moderate levels compared to HL60 and 50-80% CD123. KGla is, therefore, a relatively dual target cell population that is double positive for the antigens targeted by the CD33CD123 CAR. At about 24 hours of incubation and low effector:target (E:T) ratios ranging from about 0.5:1 to 50:1 were used. While at a low E:T ratio of about 2:1, the CD33CD123 CAR exhibited modest anti-leukemic activity about 26%, an increase in E:T ratio to 10:1 resulted in a killing of KGla of about 62% compared to GFP control (FIG. 4), signaling that the intensity of the CD33 marker may be an indicator for the efficacy of killing with HL60 presenting strongly and harnessing more CAR action than KG1a. These experiments provide evidence for the function of the whole CD33CD123 CAR against its relevant antigen presenting cell populations.

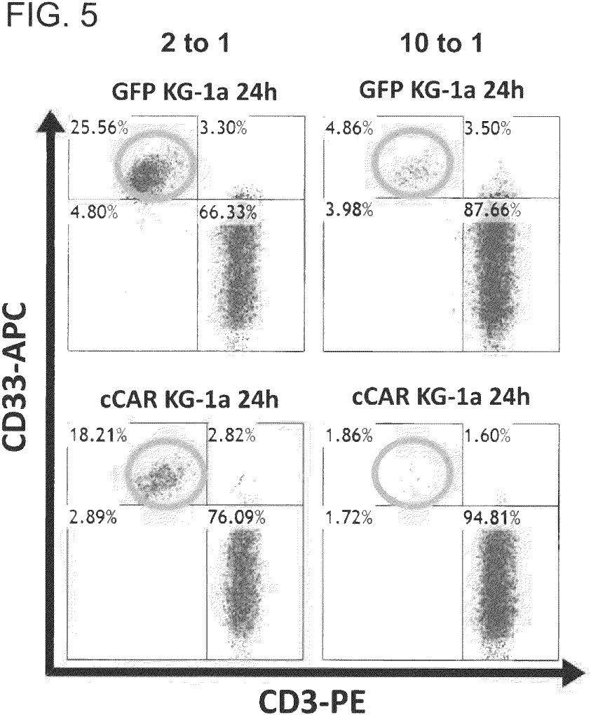

Additional compound CAR, CD33CD123-BB cCAR has been generated. This compound CAR comprises two independent units of CARs, CD33 and CD123. The first CAR comprises scFv binding to CD33 and the second CAR bears a different scFv recognizing CD123. Both CARs contain the same hinge region, transmembrane, co-stimulatory and intracellular domains. CD33CD123-BB cCAR lentiviruses were produced and their killing ability was tested in KG-1a cells. As shown in FIG. 5, there was substantial killing at a ratio of about 10:1 but it is less potent than that of CD33CD123 cCAR.

### CD33CD123 cCAR Possesses Activity Against Patient Samples Expressing CD33 and/or CD123

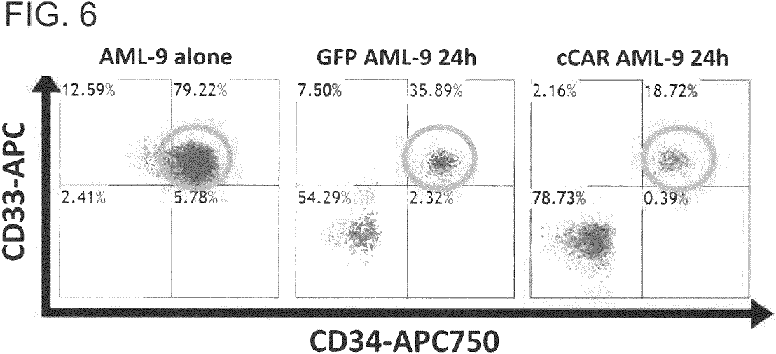

In addition to cell line experiments, studies were also conducted on patient samples in order to test the function of each individual CAR unit. An aggressive acute myeloid leukemia (AML), AML-9 was used for testing efficacy of the CD33CD123 cCAR. Due to the heterogeneity of the patient cell population, which includes multiple cell types in the AML-9 sample, leukemic blasts were gated with CD34 and CD33, as they were positive for these two markers. The depletion of this CD33+CD34+ population of leukemic cells was observed to be 48% over the GFP control at a ratio of CAR T cell:target cell (FIG. 6).

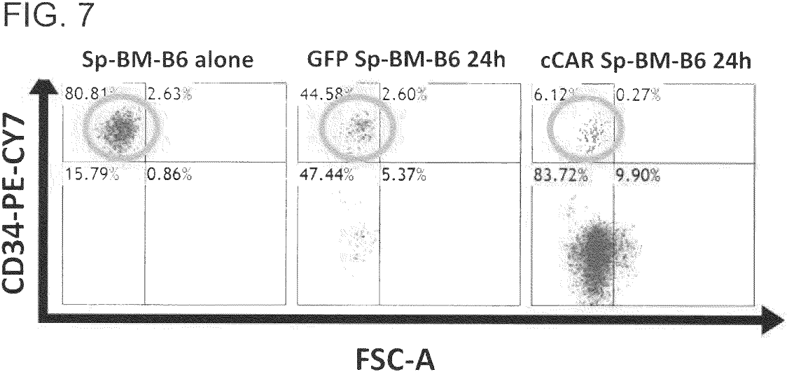

Leukemic cells that were CD123 positive and CD33 negative were also tested. For this purpose, human B cell acute lympoblastic leukemia (B-ALL) sample, Sp-BM-B6 was chosen. All leukemic blasts in this sample were CD34+CD33-, and more than about 50% positive for CD123. Depletion of the CD34+ leukemic cell population by CD33CD123 cCAR T cells was about 86% as compared to that of the GFP control (FIG. 7). Based on the cell line and human sample studies, our data strongly suggest that the compound CD33CD123 CAR is able to target leukemic cells expressing CD33 or CD123 or both.

### CD33CD123 cCAR NK Cells Targeting Leukemia Cells Expressing CD33 or CD23 or Both

Natural killer (NK) cells are CD56+ CD3- and can efficiently kill infected and tumor cells like CD8+ T cells. Unlike CD8+ T cells, NK cells launch cytotoxicity against tumors without the requirement of activation to kill cells. NK cells are safer effector cells, as they may avoid the potentially lethal complications of cytokine storms. However, the use of either CD33 or CD123 or both CAR NK cells in killing leukemias is entirely unexplored.

### Production of CD33CD123 cCAR NK Cells

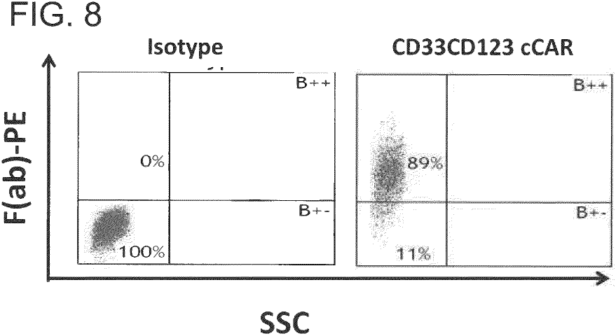

NK-92 cells were transduced with CD33CD123 CAR lentiviral supernatant in two consecutive overnight transductions with a change of retronectin- and virus-coated plates in between. The transduced cells were expanded for 3 or 4 days and then analyzed by flow cytometry for CAR expression. Cells were harvested and incubated with goat anti-mouse F(Ab′)2 at about 1:250 for about 30 minutes. Cells were washed, suspended and stained with streptavidin-PE for about 30 minutes. Cells were washed and suspended in 2% formalin, and analyzed by flow cytometry. NK-92 cells expressing CD33CD123 cCAR were then labeled as above and sorted on FACSAria, with the top 0.2% of F(Ab′)2-expressing cells collected and cultured. Subsequent labeling of sorted, expanded cells showed about 89% of NK-92 cell positive for anti-mouse F(Ab′)2 (FIG. 8).

### CD33CD123 cCAR NK Cells Efficiently Lyse or Eliminate Leukemic Cells

First, we tested the function of CD33CD123 cCAR NK-92 cells by assessing their ability to kill a HL-60 cancerous cell line in co-culture. Virtually all HL-60 cells highly express CD33 but CD123 expression in this cell line is only less than 10% (weak). Therefore, it is likely that the killing ability of CD33CD123cCAR is dependent on the ability for cCAR to properly targeting CD33.

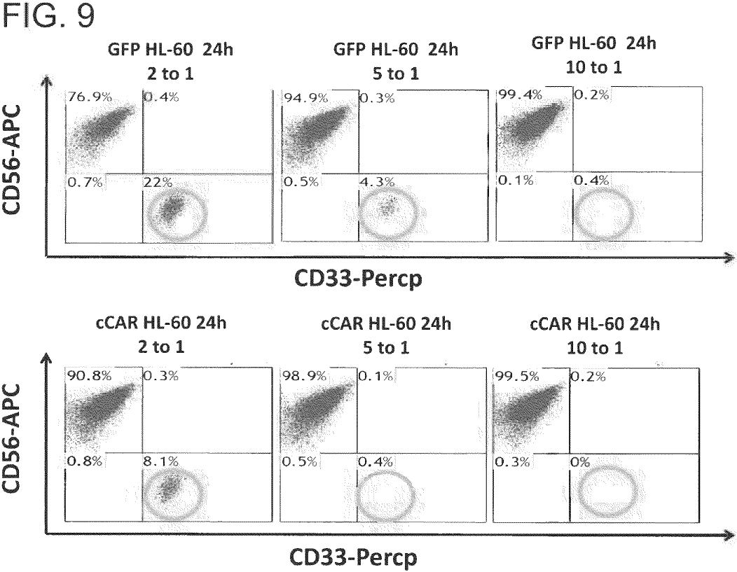

CD33CD123 cCAR NK-92 cells were co-cultured with the HL-60 cells for about 24 hours in NK cell media without IL-2. After the incubation, the CD33CD123 cCAR NK-92 cells were labeled and compared to a control of non-CAR, GFP NK-92 cells. Dramatic killing of HL-60 cells by CD33CD123 cCAR NK-92 cells was observed as compared to the control, GFP NK-92 cells. Moreover, the killing ability of CD33CD123 cCAR NK-92 cells was dose-dependent, with a about 10 to 1 ratio of about 100% compared to the control (FIGS. 9 and 11).

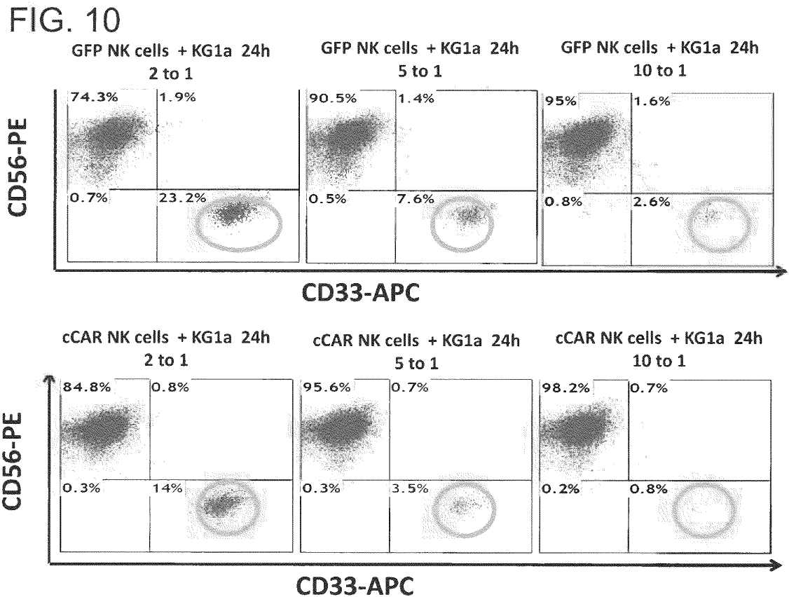

A second co-culture experiment using the myeloid leukemia cell line was performed using KG1a, which expresses CD33 in all cells but at a moderate level compared to that of HL-60. The CD123 antigen is expressed in about 50-80% of KG1a cells. The experimental design was similar to the first experiment of the HL-60 killing assay described above, with the same incubation time, effector:cancer cell ratios and GFP NK-92 cell controls. Results show a remarkable killing of KGla cells by CD33CD123 cCAR NK-92 cells in a dose-dependent manner as compared to the GFP NK-92 cell control. At a ratio of effector: target of 10:1, killing of KGla cells by CD33CD123 cCAR NK-92 cells was about 85% as compared to that of GFP control (FIGS. 10 and 11).

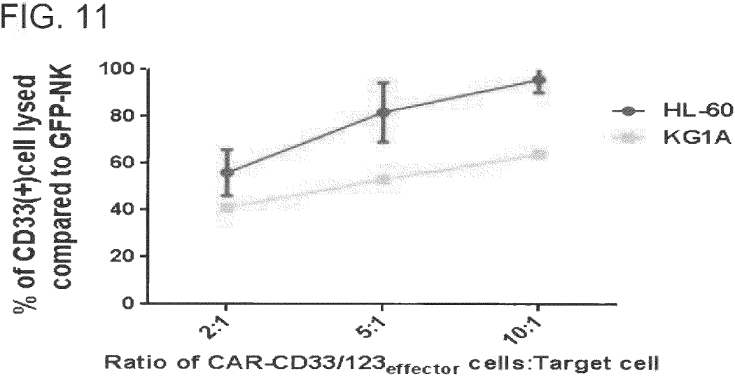

Analysis of KGla cells showed two different populations, CD33+CD123- and CD33+CD123-. FIG. 11 showed a dose dependent increase in cell killing seen in both populations. Surprisingly, the double positive population showed a higher efficient killing for each increased ratio, suggesting a possible synergistic effect of two modular CARs of CD33 and CD123 (FIG. 12).

### Generation of CD19CD20, CD19CD22, CD19CD138 cCARs

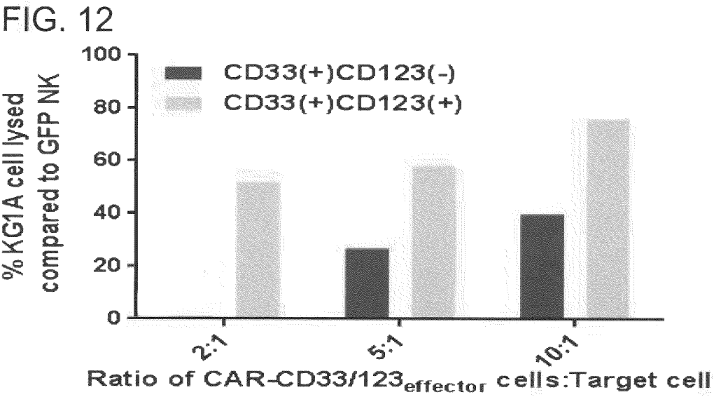

The three cCARs have been generated (FIG. 13) using the similar strategy to that of the CD33CD123 cCAR described above.

### Generation of cCAR Including BCMA CS1 cCAR and BCMA CD19 cCAR for Treatment of Multiple Myeloma

Pre-clinical studies have been developed for cCARs to target surface antigens including CD38, CS1, CD138, B cell maturation antigen (BCMA) and CD38. CD19 CAR has also demonstrated some efficacy for the treatment of multiple myeloma in a phase I clinical trial. However, given that the heterogeneity of surface antigen expression commonly occurs in malignant plasma cells(Ruiz-Arguelles and San Miguel 1994), it is unlikely that a single target is sufficient to eliminate this disease. BCMA CS1 cCAR, BCMA CD19 cCAR, BCMA CD38 cCAR and BCMA CD138 cCAR were generated and the experimental design was similar to that of CD33CD123 cCAR as described above.

### Generation of cCAR Including BCMA CS1 cCAR (BC1cCAR) for Treatment of Multiple Myeloma

### Generation and Characterization of BCMA-CS1 cCAR (BC1cCAR) Construct

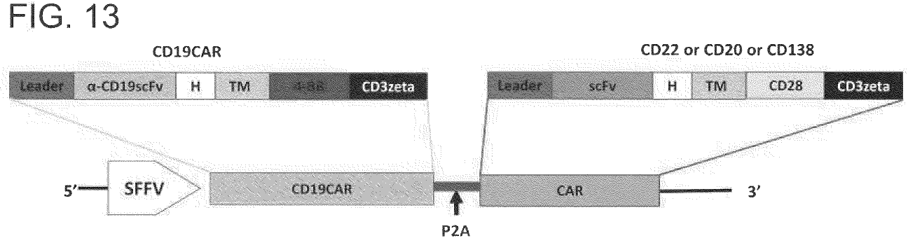

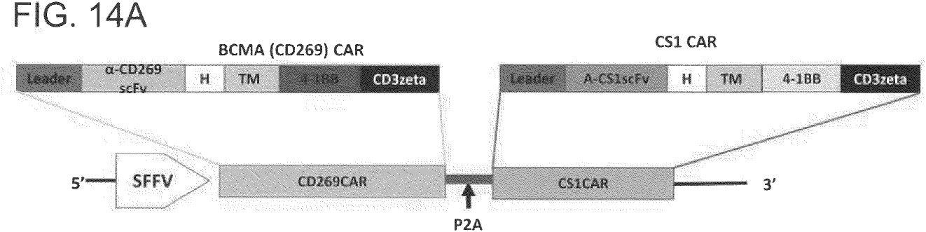

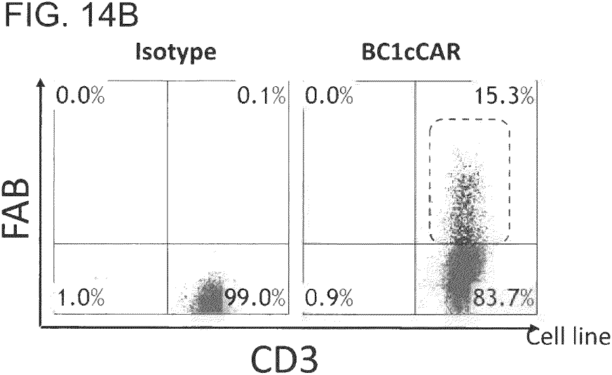

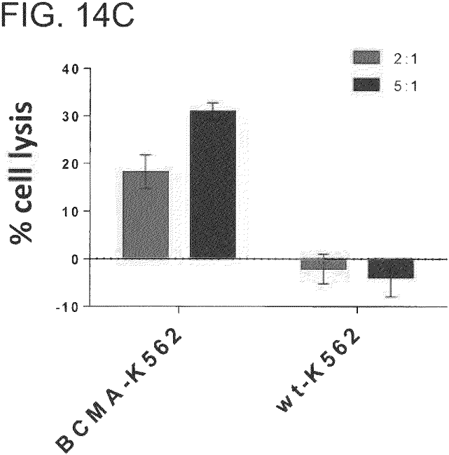

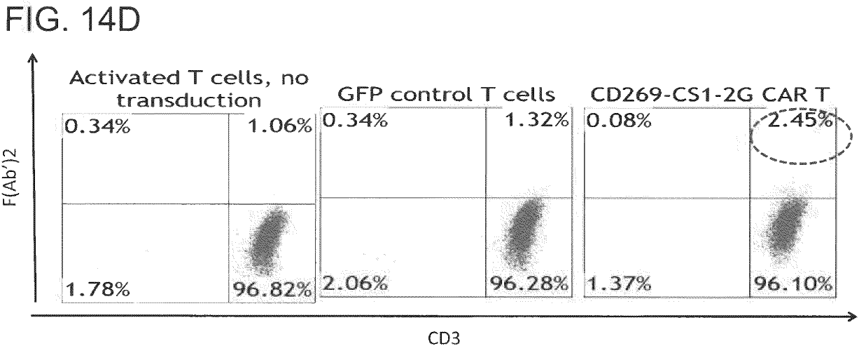

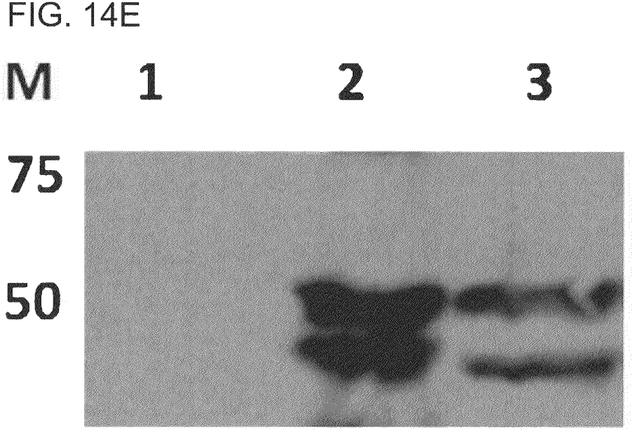

BC1cCAR’s modular design consists of an anti-CD269 (BCMA, B-cell maturation antigen) single-chain variable fragment (scFv) region fused to an anti-CD319 (CS1) scFv by a self-cleaving P2A peptide, CD8-derived hinge (H) and transmembrane (TM) regions, and tandem 4-1BB co-activation domains linked to the CD3ζ signaling domain (FIG. 14A). A strong spleen focus forming virus promoter (SFFV) and a CD8 leader sequence were used for efficient expression of the BC1cCAR)CAR molecule on the T-cell surface. Two unit CARs use same co-stimulatory domain, 4-1BB. Transfected BC1cCAR HEK293T cells were subjected to Western blot analysis in order to confirm the compound construct. Immunoblot with an anti-CD3ζ monoclonal antibody showed bands of predicted size for the compound CAR CD3ζ fusion protein (FIG. 14E). Importantly, two distinct bands of similar intensity were observed on the blot signaling the successful high cleavage action of the P2A peptide as expected. No CD3ζ expression was seen for the GFP control vector as expected.

### Generation of BC1cCAR (cCAR) T-Cells

T-cells isolated from umbilical cord blood (UCB) buffy coats were transduced with BC1cCAR lentivirus after 2 days of activation. Two unit CARs used the same co-stimulatory domain, 4-1BB. BC1cCAR’s transduction efficiency was determined to be about 15% as determined by flow cytometry (FIG. 14B). BC1cCAR T-cells were first tested on a CML (chronic myeloid leukemia) cell line negative for the myeloma markers, BCMA and CS1. As expected, there was no lysis from either control T-cells or BC1cCAR T-cells against wild-type K562 (FIG. 14C). BCMA-K562 (Kochenderfer, NIH) were K562 cells transduced with BCMA expressing cDNA to express BCMA at >80% of the cell population. BC1cCAR T-cells were co-cultured with this cell line at E:T ratios of 2:1 and 5:1 and show over 30% lysis as compared to control (undetectable)(FIG. 14C). These results are compatible with other cultures performed on antigen-transduced cell lines for other CARs, such as CS1CAR T-cells.

However, when BCMA-CS 1-2G (a cCAR) used a different co-stimulatory domain, either 4-BB or CD28 for each unit, rare surface CAR expression was detected, which indicate that an appropriate selection of a co-stimulatory domain may be important for ensuring the surface CAR expression on T cells (FIG. 14D). Although protein was detected in HEK cells by Western blotting (FIG. 14E), we were unable to detect surface expression in activated T cells transduced with CD269-CS1-2G lentiviral supernatant. This may be due to an inability to export the expressed protein to the cell membrane. In future, we may need to optimize the sequence of this construct to allow for greater cell surface expression.

### BC1cCAR T-Cells Specifically Lyse BCMA⁺ and CS1⁺ Cell Lines

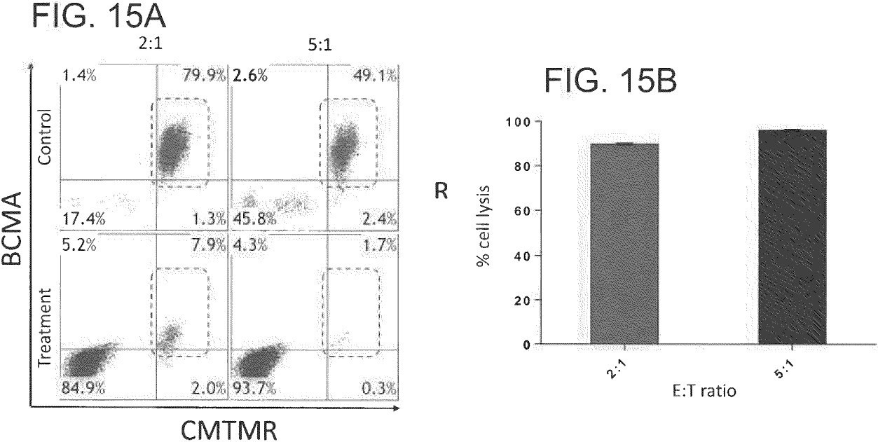

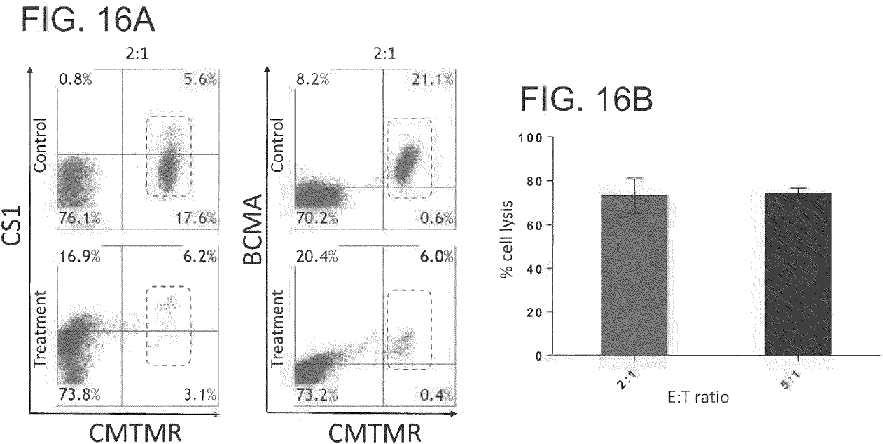

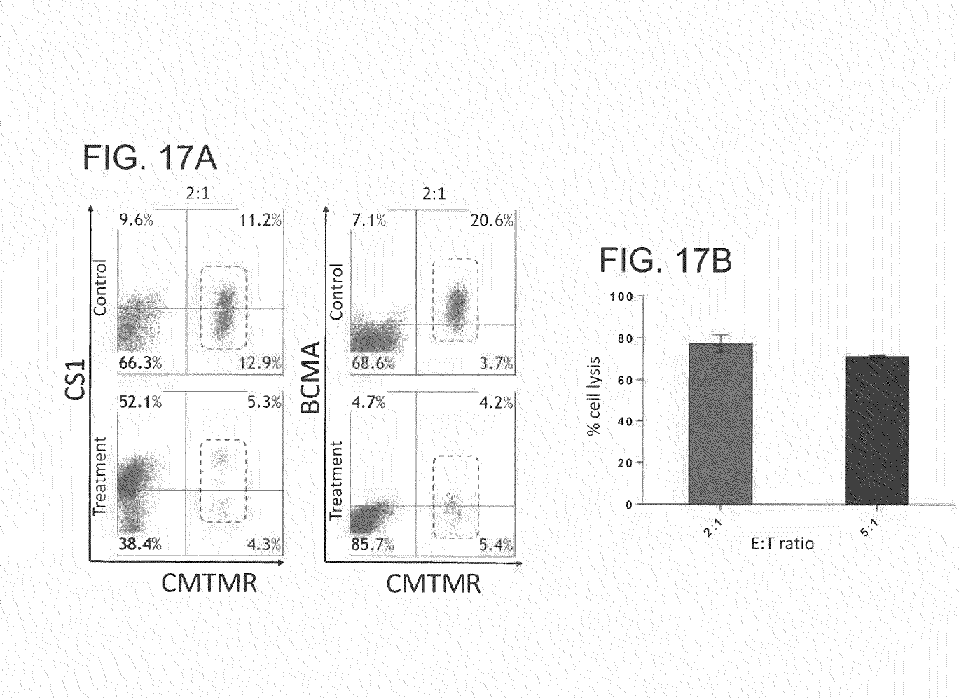

To assess the cytotoxicity ability of BC1cCAR T-cells, we conducted co-culture assays with myeloma cell lines: MM1S (BMCA⁺ CS1⁺⁾, RPMI-8226 (BCMA⁺ CS1⁻), and U266 (BCMA⁺ CS1^{dim}). The ability of the BC1cCAR T-cells to lyse the target cells was quantified by flow cytometry analysis, and target cells were stained with Cytotracker dye (CMTMR). In 24 hour co-cultures, the BC1cCAR exhibited virtually complete lysis of MM1S cells, with over 90% depletion of target cells at an E:T ratio of 2:1 and over 95% depletion at an E:T of 5:1 (FIG. 15). In RPMI-8226 cells, BC1cCAR lysed over 70% of BCMA⁺ target cells at an E:T ratio of 2:1, and over 75% at an E:T of 5:1(FIG. 16). In 24 hour co-culture with U266 target cells, BC1cCAR lysed 80% of BCMA⁺U266 cells at an E:T ratio of 2:1, reaching saturation (FIG. 17).

### BC1cCAR T-Cells Specifically Target BCMA⁺ and CS1⁺ Populations in Primary Patient Myeloma Samples

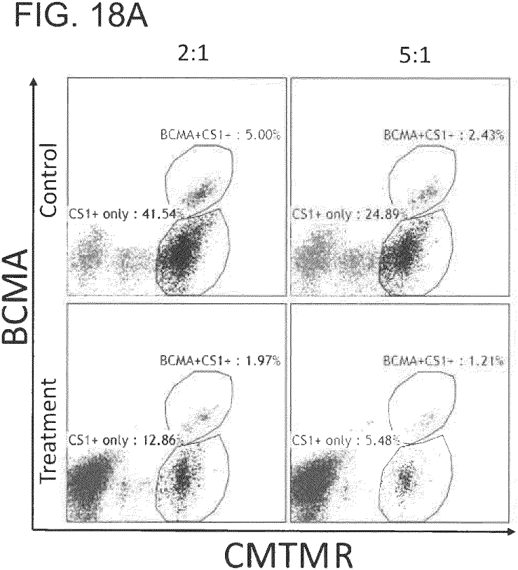

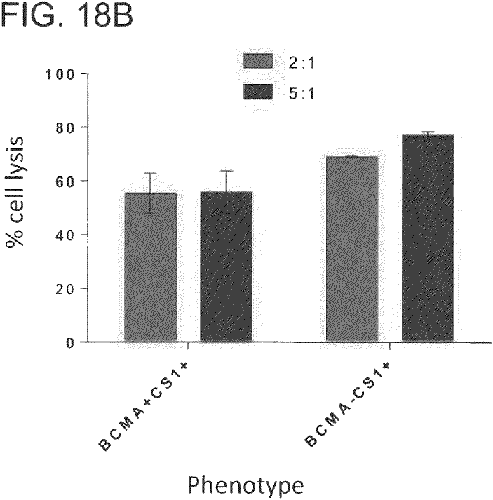

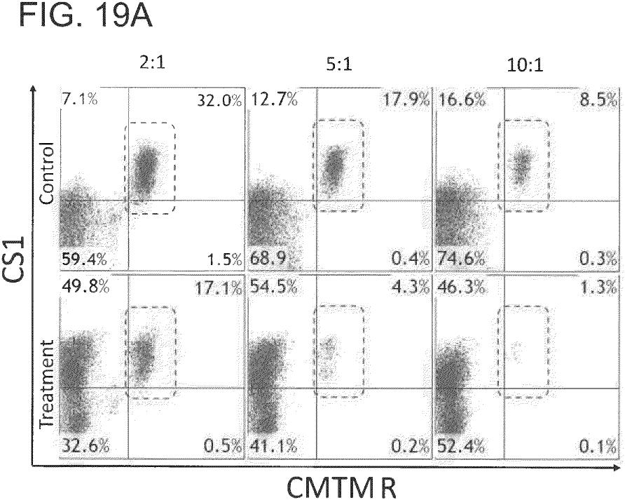

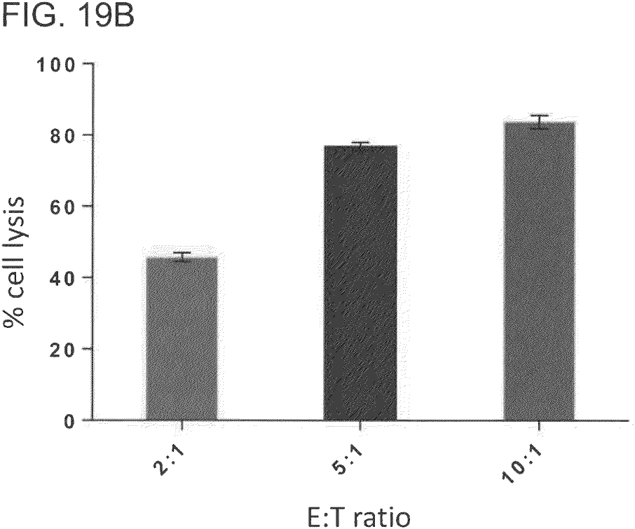

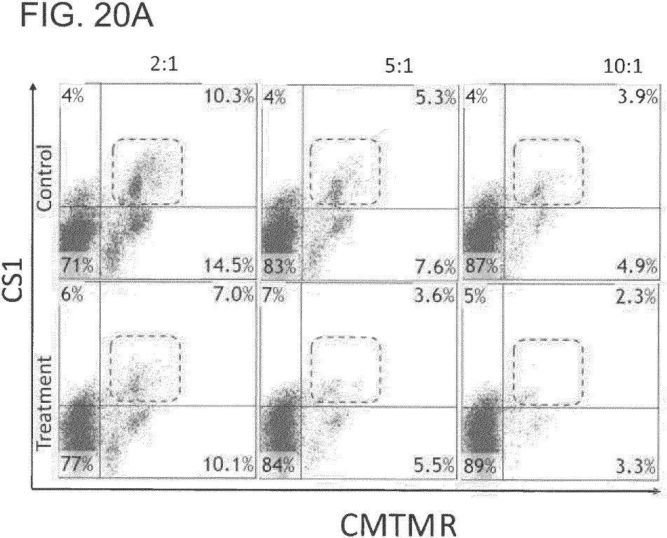

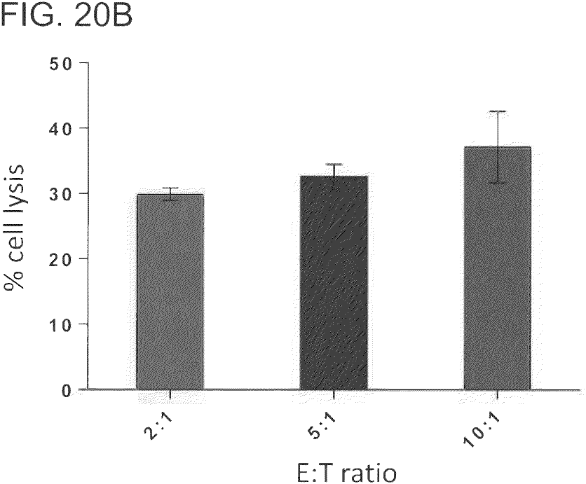

Flow cytometry analysis of the MM10-G patient sample reveals distinct and consistent BCMA⁺ and CS1⁺ population subsets (FIG. 18). MM7-G sample shows a complete BCMA⁺ CS1⁺ phenotype while MM11-G exhibits a noisy BCMA^{dim} CS1^{dim} phenotype likely attributable to its property of being a bone-marrow aspirate. After 24 hours, BC1cCAR T-cells show robust ablation of the MM7-G primary patient sample, with over 75% lysis at an E:T ratio of 5:1, increasing to over 85% at 10:1 (FIG. 19). Against the MM11-G (FIG. 20), BC1cCAR T-cells were able to lyse over 45% of BCMA⁺ CS1⁺ population at an E:T of 10:1.

BC1cCAR show targeted and specific lysis ability, by significantly ablating both the BCMA⁺CS1⁺ and the BCMA⁻CS1⁺ population subsets in MM10-G co-cultures over 24 hours. At an E:T ratio of 2:1, BC1cCAR T-cells ablate over 60% of the BCMA⁺ CS1⁺ population, and 70% of the CS1⁺ only population. At an E:T ratio of 5:1, the ablation of CS1⁺ only population increases to 80% (FIG. 18).

### BC1cCAR T-cells Exhibit Significant Control and Reduction of Tumor In Vivo

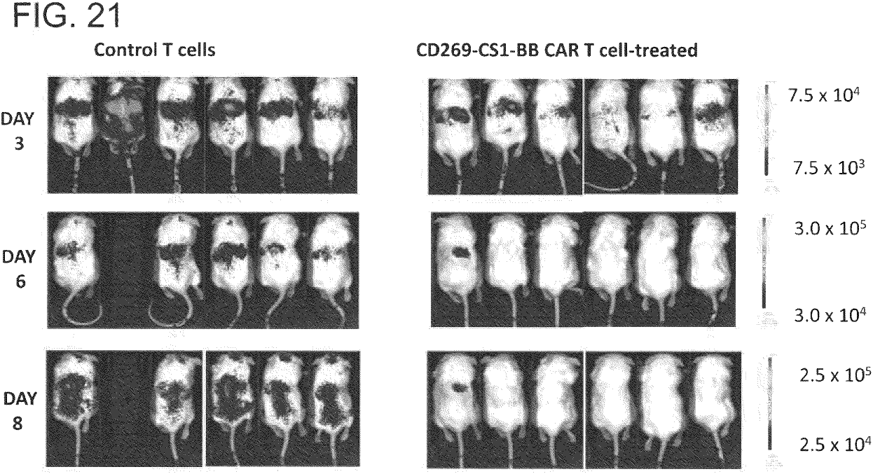

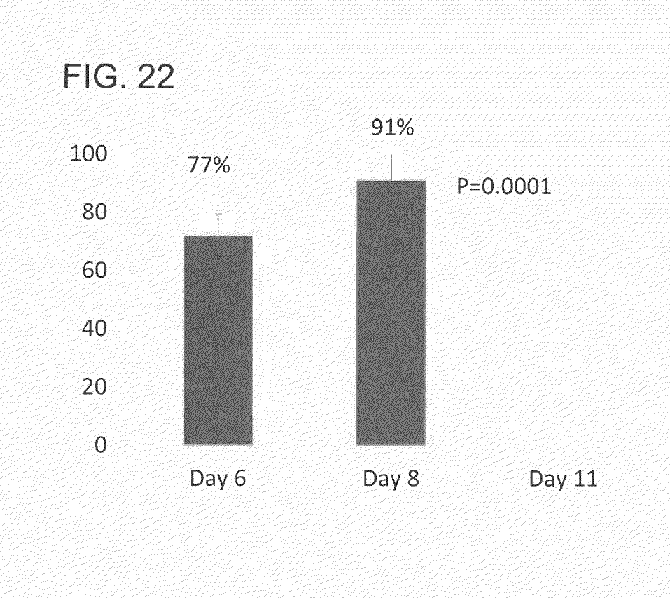

In order to evaluate the in vivo anti-tumor activity of BC1cCAR T cells, we developed a xenogeneic mouse model using NSG mice sublethally irradiated and intravenously injected with luciferase-expressing MM.1S cells, a multiple myeloma cell line, to induce measurable leukemic formation. Three days following tumor cell injection, mice were intravenously injected with 8 × 10⁶ BC1cCAR T cells or vector control cells in a single dose. On days 3, 6, and 8, mice were injected subcutaneously with RediJect D-Luciferin (Perkin Elmer) and subjected to IVIS imaging to measure tumor burden (FIG. 21). Average light intensity measured for the BC1cCAR T cells injected mice was compared to that of vector control injected mice in order to determine the percentage of tumor cells in treated versus control mice (FIGS. 21 and 22). Unpaired T test analysis revealed an extremely significant difference (P=0.0001) between the two groups by day 8 with less light intensity and thus less tumor burden in the BC1cCAR T cells injected group compared to control (p <0.0001). On day 1, and every other day afterwards, tumor size area was measured and the average tumor size between the two groups was compared (FIG. 21). In summary, these in vivo data indicate that CD269-CS1-BBCAR T cells significantly reduce tumor burden in MM.1S-injected NSG mice when compared to vector control NK control cells.

### CD45 CAR Therapy

Three pairs of sgRNA are designed with CHOPCHOP to target the gene of interest. Gene-specific sgRNAs are then cloned into the lentiviral vector (Lenti U6-sgRNA-SFFV-Cas9-puro-wpre) expressing a human Cas9 and puromycin resistance genes linked with an E2A self-cleaving linker. The U6-sgRNA cassette is in front of the Cas9 element. The expression of sgRNA and Cas9puro is driven by the U6 promoter and SFFV promoter, respectively (FIG. 23).

The following gene-specific sgRNA sequences were used and constructed, In a non-limiting embodiment of the invention, exemplary gene-specific sgRNAs have been designed and constructed as set forth below:

CD45 sgRNA construct::

| | |
|---|---|
| Lenti-U6-sgCD45a-SFFV-Cas9-puro | GTGGTGTGAGTAGGTAA |
| Lenti-U6-sgCD45b-SFFV-Cas9-puro | GAGTTTTGCATTGGCGG |
| Lenti-U6-sgCD45c-SFFV-Cas9-puro | GAGGGTGGTTGTCAATG |

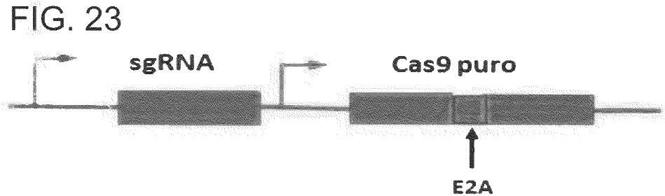

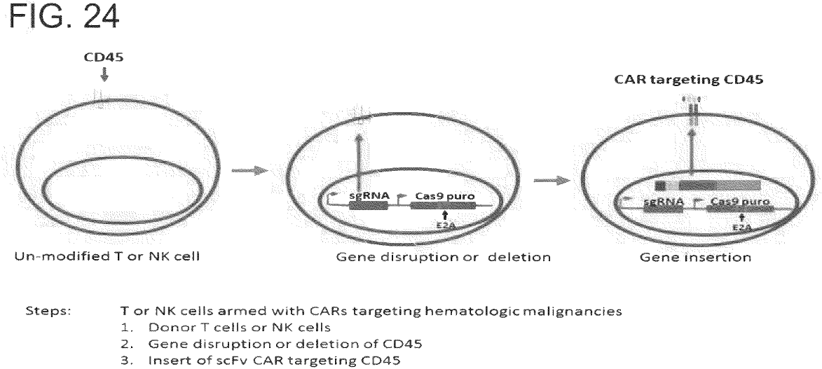

Figure 24 shows steps of generation of CD45 CAR T or NK cell targeting hematologic malignancies.

### CRISPR/Cas Nucleases Target to CD45 on NK Cells

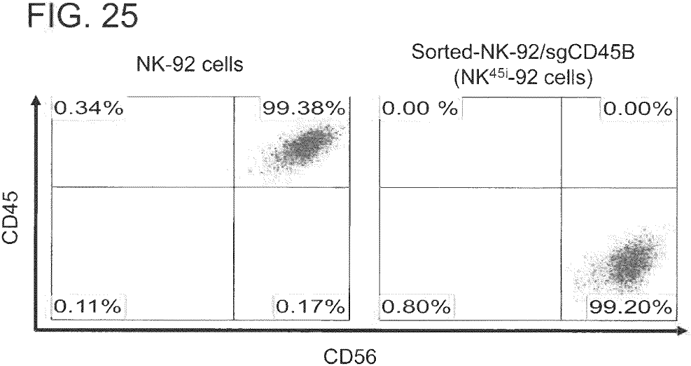

Lentiviruses carried gene-specific sgRNAs were used to transduce NK-92 cells. The loss of CD45 expression on NK-92 cells was determined by flow cytometry analysis. The CD45 negative population of NK-92 cells was sorted and expanded (FIG. 25). The sorted and expanded CD45 negative NK-92 cells were used to generate CD45CAR NK cells. The resulting CD45CAR NK cells were used to test their ability of killing CD45+ cells.

### Functional Characterization of CD45 Inactivated NK-92 Cells (NK⁴⁵ⁱ-92) After CRISPR/Cas Nucleases Target

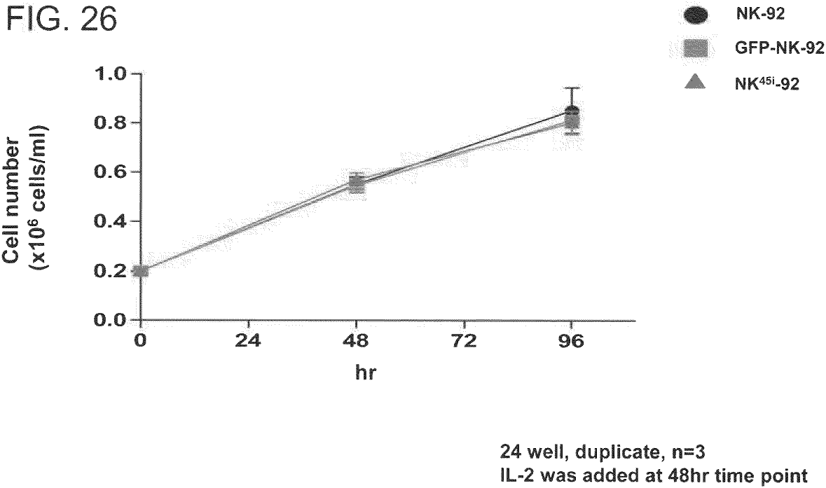

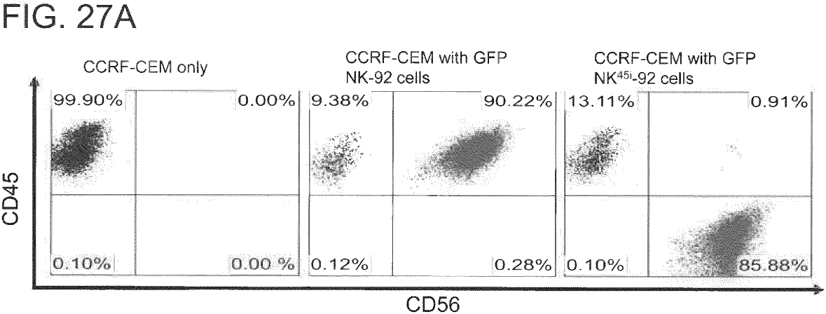

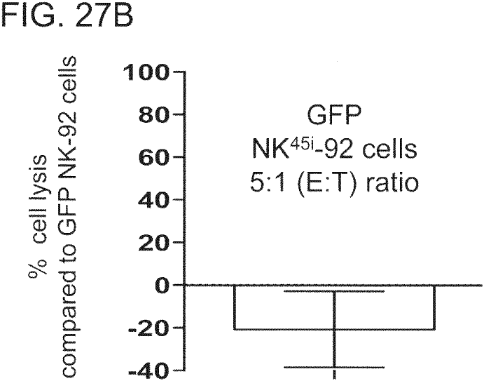

We demonstrated that, following CRISPR/Cas nuclease inactivation of CD45, the growth of NK⁴⁵ⁱ-92 cells was similar to that of the wild NK-92 cells (FIG. 26). Inactivation of CD45 did not significantly affect the cell proliferation of NK-92. In addition, we showed that the lysis ability of NK⁴⁵ⁱ-92 cells was compatible to that of wild type, NK-92 when cells were co-cultured with leukemic cells, CCRF (FIG. 27).

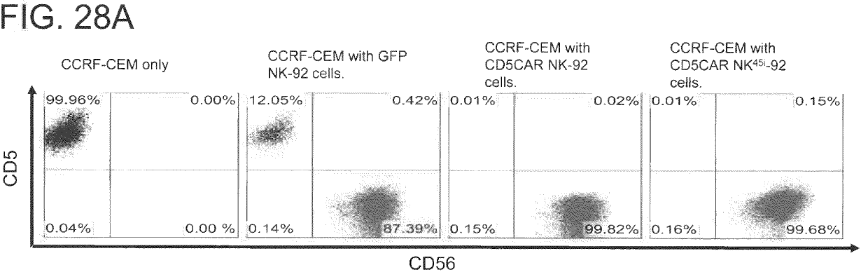

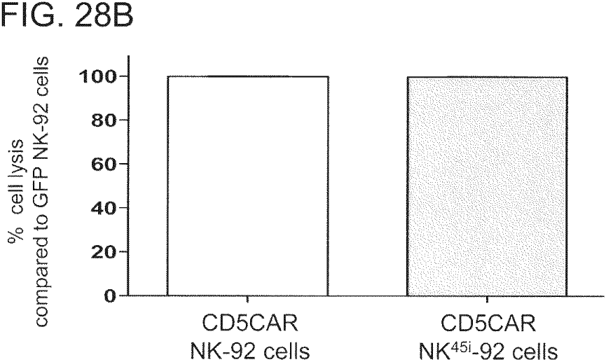

To demonstrate that CD45 -inactivated NK-92 was compatible with CAR lysis, NK⁴⁵ⁱ-92 cells and their wild type, NK-92 were transduced with lentiviruses expressing CD5CAR or GFP. The resulting CD5CAR NK⁴⁵ⁱ-92 cells and GFP NK⁴⁵ⁱ-92 were sorted by FACS, and used to compare their ability of killing targeted cells. CD5CAR NK⁴⁵ⁱ-92 cells displayed the ability of robustly killing CD5 target leukemic cells at ratios (E:T), 2:1 and 5:1 when they were co-cultured with CCRF-CEM cells. We showed that there was a similar efficacy of elimination of CCRF-CEM cells in vitro between CD5CAR NK⁴⁵ⁱ-92 and CD5 CAR NK-92 cells (FIG. 28). This suggests that the loss of CD45 expression does not diminish the anti-tumor activity of CAR NK-cells.

### Generation of CD45CAR Construct

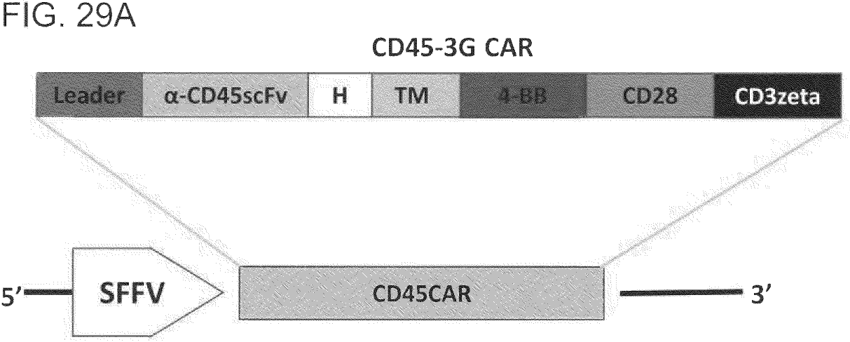

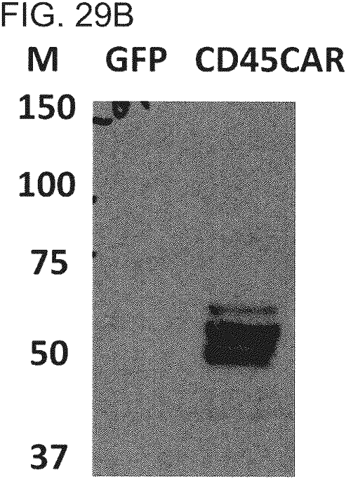

We next investigate that CD45CAR in NK⁴⁵ⁱ-92 cells response to the CD45 antigen in leukemic cells. We generated CD45CAR. CD45CAR consists of an anti-CD45 single-chain variable fragment (scFv) region, CD8-derived hinge (H) and transmembrane (TM) regions, and tandem CD28 and 4-1BB co-activation domains linked to the CD3ζ signaling domain (FIG. 29A). A strong spleen focus forming virus promoter (SFFV) and a CD8 leader sequence were used. CD45CAR protein was characterized by Western blot of HEK293-FT cells transfected with CD45CAR lentiviral plasmid with appropriate vector control. Additionally, anti-CD3zeta monoclonal antibody immunoblots revealed bands of predicted size for the CD45CAR protein with no bands observed in vector control (FIG. 29B).

### CD45CAR NK⁴⁵ⁱ-92 NK Cells

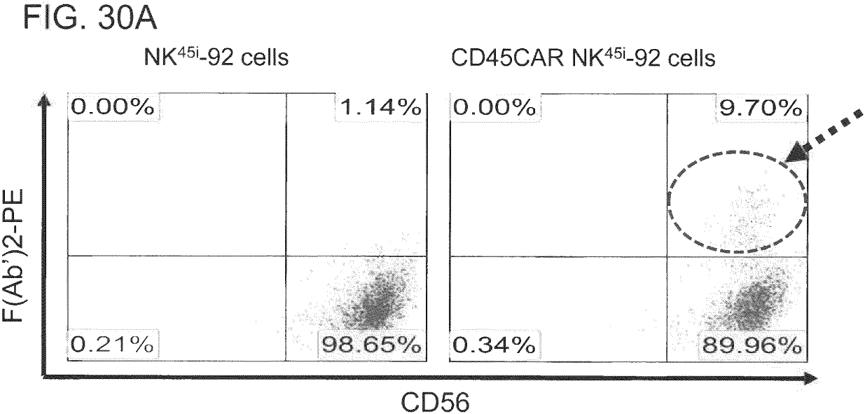

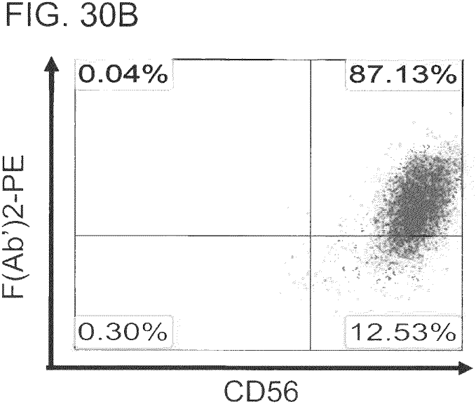

Following fluorescence-activated cell sorting (FACS) to enrich for NK⁴⁵ⁱ-92 cells, CD45CAR NK-92 transduction efficiency was determined to be 87%, as determined by flow cytometry (FIG. 30) after sorting. After FACS collection of NK⁴⁵ⁱ-92 cells, CD45CAR expression levels remained consistently stable for at least 10 passages.

### CD45CAR NK⁴⁵ⁱ-92 Cells Specifically Lyse CD45+ Leukemic Cells

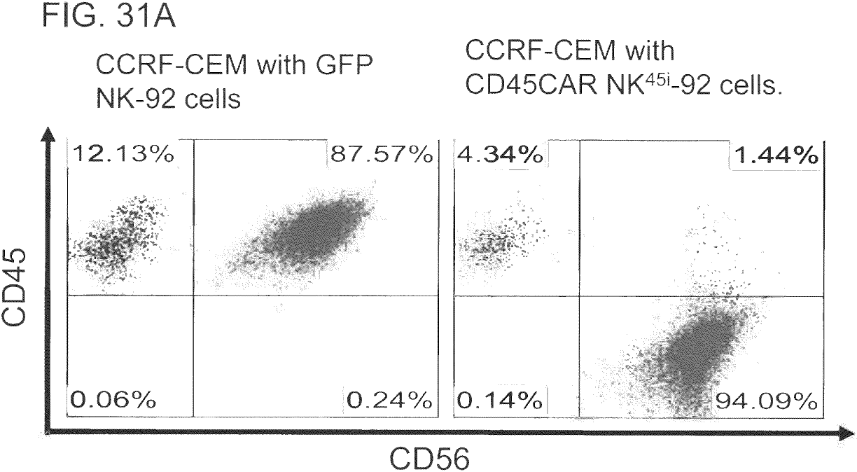

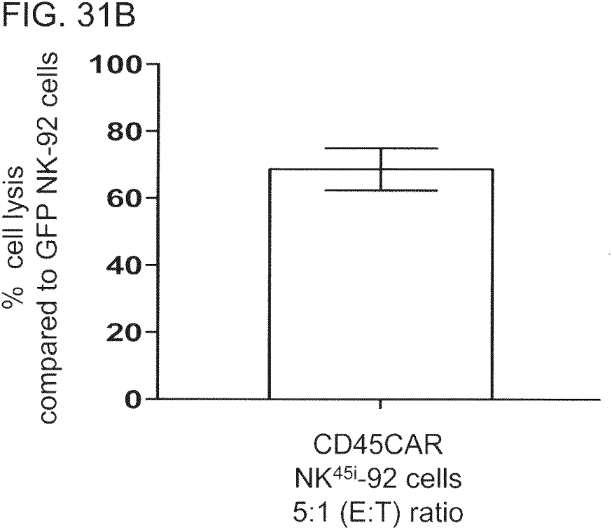

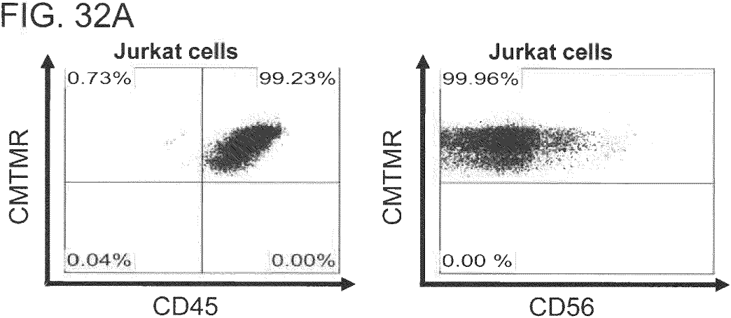

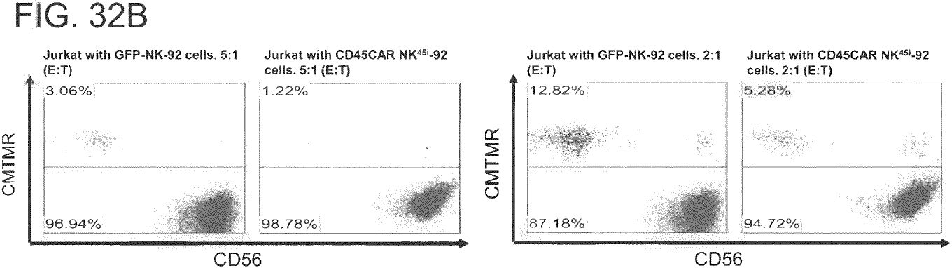

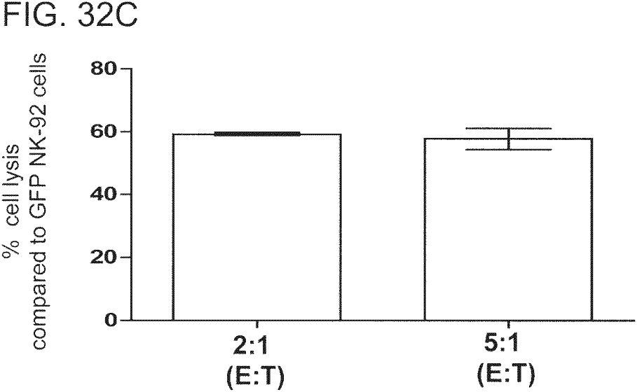

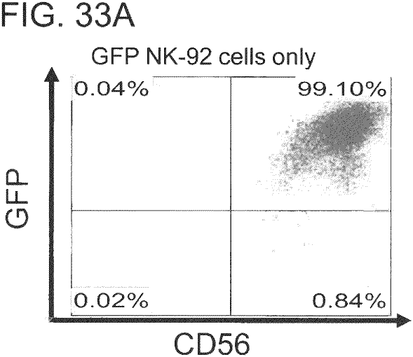

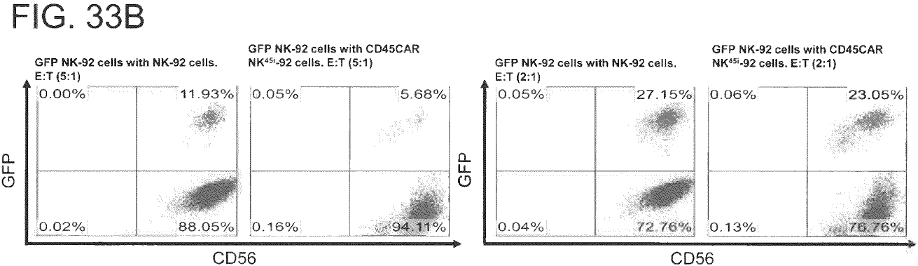

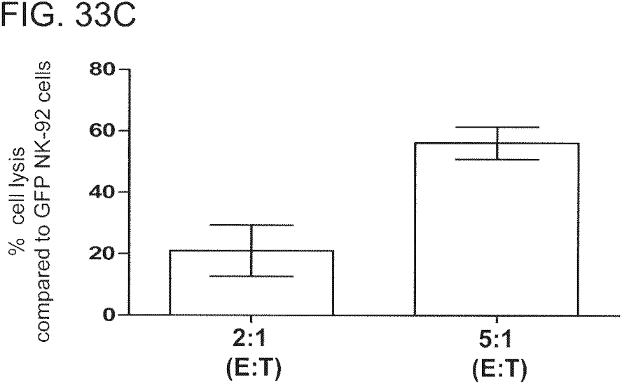

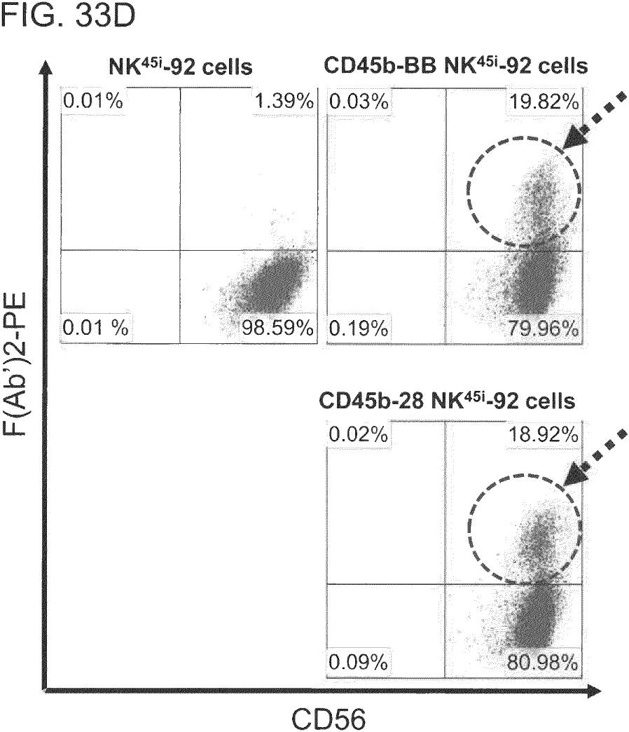

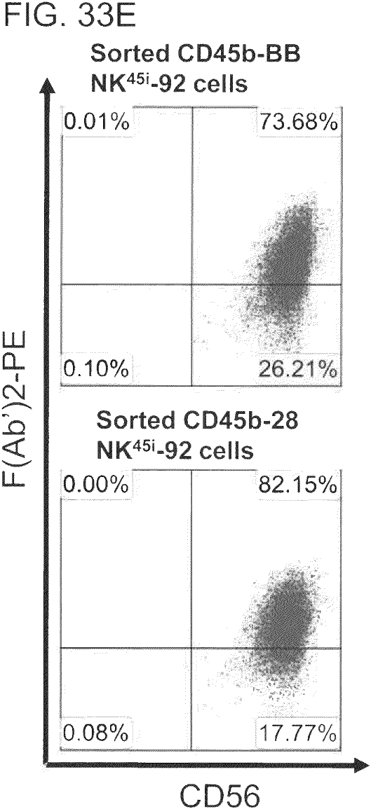

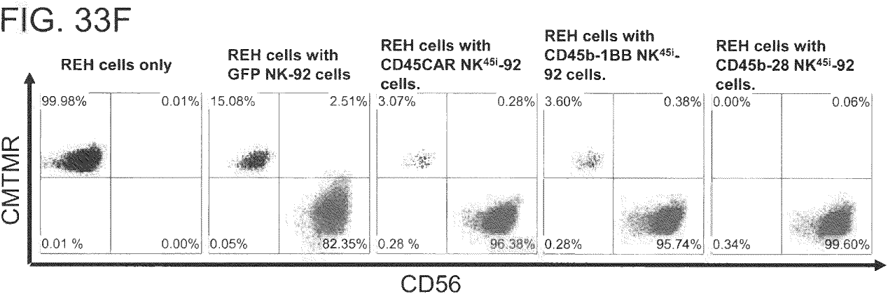

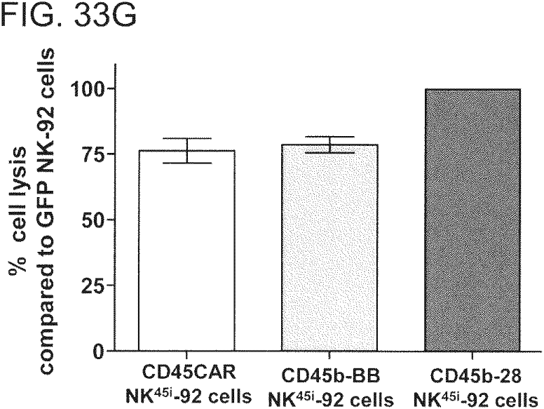

To assess CD45CAR NK⁴⁵ⁱ-92 anti-leukemic activity, we conducted co-culture assays using T-ALL cell lines, CCRF-CEM and Jurkat, and NK cell line and NK--92 cells since they all express CD45 (FIGS. 31, 32 and 33). We demonstrated that CD45CAR NK⁴⁵ⁱ-92 cells consistently displayed robust lysis of leukemic cells. Following 6-hour incubation at a low effective to target cell (E:T ratio 5:1), CD45CAR NK⁴⁵ⁱ-92 cells effectively lysed more than 60% of CCRF-CEMcells (FIG. 31). After 6-hour co-culture, CD45CAR NK⁴⁵ⁱ-92 cells were also able to eliminate about 60% of Jurkat cells at a ratio of E:T, 2:1 or 5:1(FIG. 32). After 6 hours of co-culture, CD45CAR NK⁴⁵ⁱ-92 cells efficiently lysed 20% CD45 positive NK-92 cells at an E:T ratio of 2:1, with close to 60% lysis at an E:T of 5:1 (FIGS. 33A-33C).

To further analyze the CD45 target for hematologic malignancies, we also generated additional two CARs: CD45-28 and CD45-BB, and the lentiviruses expressing CD45-28 or CD45-BB CAR were used to transduce NK45i -92 cells. CD45-28 and CD45-BB CARs contain a new anti-CD45 scFv, which is different from that of CD45CAR described above. CD45-28 CAR uses a CD28 co-stimulatory domain while the CD45-BB bears a 4-BB co-stimulatory domain. Both CARs use the CD8-derived hinge (H), transmembrane (TM) regions and CD3ζ signaling domain. CD45CARs displayed robust lysis of B acute lymphoblastic cell line, REH. CD45CAR NK45i-92 cells lysed about 76% REH cells. CD45b-BB CAR NK45i-92 cells and CD45b-28 CAR NK45i-92 cells showed about 79% and 100% lysis of REH cells, respectively compared to control GFP NK-92 cells (FIGS. 33D-G). CD45b-28 CAR NK45i-92 cells exhibited the highest ability of lysis of REH cells.

### IL15 and Its Receptor in Enhancing CAR T and NK Cell Functions

Recent studies have demonstrated that T cell persistence correlates well with CAR T cell therapeutic efficacy. Recent trials demonstrate that potent and persistent antitumor activity can be generated by an infused small number of CAR T cells indicative that quality rather than quantity of infused products is more important in contributing to the anti-tumor activity. Interleukin (IL)-15 is a cytokine that promotes the development and hemostasis of lymphocytes. Increased levels of IL-15 promote T-cell proliferation and enhance T cell effector response. Data from recent studies have shown that IL-15 is crucial for the generation and maintenance of memory CD8 T-cells, one of the key factors associated with anti-tumor activity. IL-15 binds the IL-15 receptor alpha chain (also called IL15RA or RA) contributing to IL-15-mediated effects such as T-cell survival, proliferation and memory T cell generation.

IL-15RA binds the βγ complex in the surface of T cells and IL15 signals by binding with this IL-15RA/ βγ complex on the cell surface of T cells and other types of cells.

Recent data have shown that while transfection of IL-15 alone does not significantly influence T-cell function, transfection of IL-15/1IL-15RA allows T cells to survive and proliferate autonomously.

The efficacy of administered IL-15 alone may be limited by the availability of free IL-15RA and its short half-life. Administration of soluble IL-15/RA complexes greatly enhanced II-15 half-life and bioavailability in vivo. Therefore, treatment of mice with this complex, but not with IL-15 alone results in robust proliferation and maintenance of memory CD8 T cells and NK cells. Recent studies have shown that a portion of the extracellular region of IL-15RA called sushi domain is required for its binding of IL15 (WEI et al., J. Immunol., vol.167(1), p:277-282, 2001). The IL-15/RA fusion protein or IL-15/sushi fusion protein containing the linker is more potent than IL-15 and soluble IL-15RA alone. The combination of IL-15/RA or IL-15/sushi can maximize IL-15 activity. However, it is unclear if a design incorporating both CAR and Il-15/RA or IL15/sushi in the same construct maintains its desired biological properties in T or NK cells as insert sequence length is able to affect transfection efficiency and gene expression levels.

The present disclosure provides an engineered cell having both CAR and IL15/RA or IL15/sushi in a single construct. In some embodiments, the disclosure includes methods to generate higher virus titer and use a stronger promoter to drive both CAR and IL15/RA or IL-15/sushi.

In some embodiments, the present disclosure provides an engineered cell having: (1) a CAR targeting an antigen including, but not limited to, CD4, CD2, CD3, CD7, CD5, CD45, CD20, CD19, CD33, CD123, CS1, and B-cell mature antigen (BCMA); and (2) IL-15; (3) IL15RA (RA) or sushi. In further embodiments, CAR comprises chimeric antigen receptor, one or more of co-stimulatory endodomains, such as CD28, CD2, 4-1BB and OX40 and intracellular domain of CD3 zeta chain. In further embodiments, a strong promoter can be, but is not limited to, SFFV. CARs, IL-15/RA or sushi and inducible suicide gene (“safety switch”), or a combination can be assembled on a vector, such as a lentiviral vector, adenoviral vector and retroviral vector or a plasmid. The introduction of “safety switch” could significantly increase safety profile, and limit on-target or off-tumor toxicities of CARs.

### Characterization of CD4IL15RA-CAR

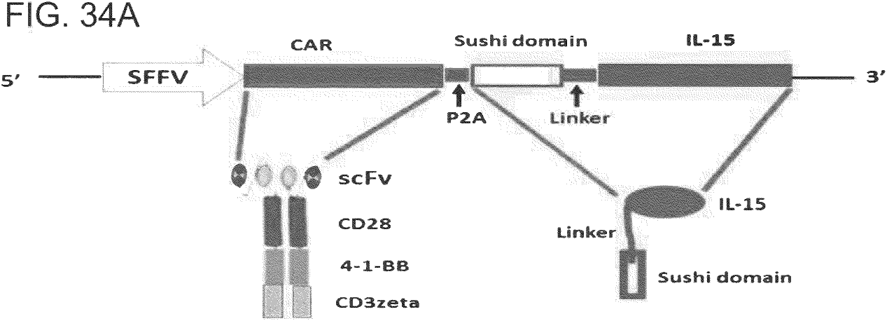

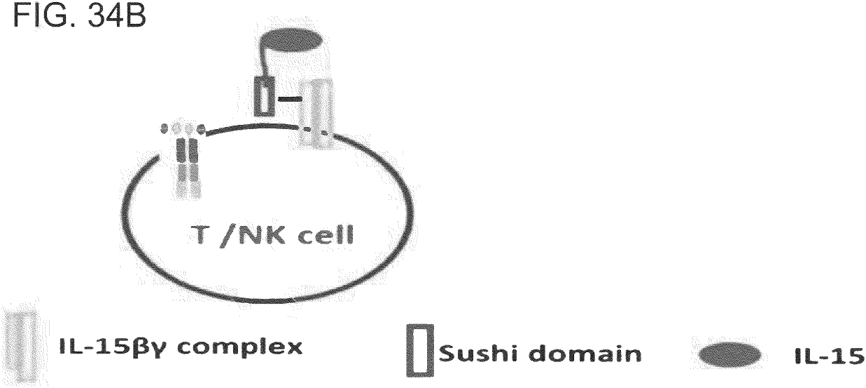

The CD4IL15RA-CAR has been generated and it contains the third generation of CD4CAR linked to IL15RA (FIG. 34). A combination of CAR, (third generation), sushi/IL-15 is assembled on an expression vector and their expression is driven by the SFFV promoter (FIG. 34). CAR with sushi/IL-15 is linked with the P2A cleaving sequence. The sushi/IL-15 portion is composed of IL-2 signal peptide fused to sushi domain and linked to IL-5 via a 26-amino acid poly-proline linker (FIG. 34).

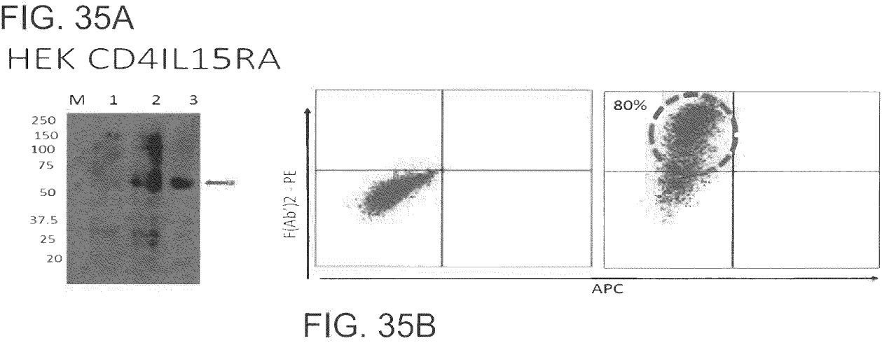

To verify the CD4IL15RA construct, HEK293FT cells were transfected with lentiviral plasmids for either GFP (control) or CD4IL15RA. Approximately 60 hours after transfection, both HEK-293FT cells and supernatant were collected. Cells were lysed in RIPA buffer containing protease inhibitor cocktail and electrophoresed. The gel was transferred to Immobilon FL blotting membrane, blocked, and probed with mouse anti-human CD3z antibody at 1:500. After washes, membrane was probed with goat anti-mouse HRP conjugate, washed, and exposed to film following treatment with HyGlo HRP substrate. The CD4IL15RA-CAR was successfully expressed in HEK 293 cells (Lane 2, FIG. 35a, as shown next to recombinant IL-15 protein in Lane 3 (arrow). The CD4IL15RA-CAR lentiviral supernatant was further examined by the transduction of fresh HEK-293 cells (FIG. 35a). HEK-293 cells were transduced with either GFP or CD4IL15RA-CAR viral supernatant from transfected HEK-293FT cells. Polybrene was added to 4 uL/mL. Media was changed after 16 hours and replaced with media containing no viral supernatant or polybrene. Three days after transduction, cells were harvested and stained with goat-anti-mouse F(Ab′)2 antibody at 1:250 for 30 minutes. Cells were washed and stained with streptavidin-PE conjugate at 1:500, washed, suspended in 2% formalin, and analyzed by flow cytometry. FIG. 34b shows that HEK-293 cells that were transduced with the CD4IL15RA-CAR lentivirus were 80% positive for F(Ab)2-PE (circled, FIG. 35b), while transduction with GFP control lentivirus was minimal for F(Ab)2-PE (FIG. 35b,left).

### Production of CD4IL15RA-CAR NK Cells

NK-92 cells were transduced with CD4IL15RA-CAR lentiviral supernatant. After 5 days incubation, cells were harvested and incubated with goat anti-mouse F(Ab′)2 at 1:250 for 30 minutes. Cells were washed, suspended and stained with streptavidin-PE for 30 minutes. Cells were washed and suspended in 2% formalin, and analyzed by flow cytometry, resulting in nearly 70% of the transduced cells expressing CD4IL15RA-CAR (circled, FIG. 36). Further experimental tests for CD4IL15RA-CAR will include leukemia/lymphoma killing assays in vitro and vivo, and comparison of target killing and proliferation rates with cells transduced with CD4CAR. The inventor also used the same strategy described above to generate CD19IL15RA-CAR.

### Production of CD4IL15RA-CAR T Cells

Human umbilical cord buffy coat cells were transduced with CD4IL15RA-CAR lentiviral supernatant. After 5 days incubation, cells were harvested and incubated with goat anti-mouse F(Ab′)2 at 1:250 for 30 minutes. Cells were washed, suspended and stained with streptavidin-PE for 30 minutes. Cells were washed and suspended in 2% formalin, and analyzed by flow cytometry, resulting in 63% of the transduced cells expressing CD4IL15RA-CAR (circled, FIG. 37). Further experimental tests for CD4IL15RA-CAR will include leukemia/lymphoma killing assays in vitro and vivo, and comparison of target killing and proliferation rates with cells transduced with CD4CAR.

### CD4IL15RACAR NK Cells Were Tested for Anti-Leukemic Activity Relative to CD4CAR NK Cells In Vitro by Co-Culturing Them With the Following CD4 Positive Cell Lines: Karpas 299 and MOLT4

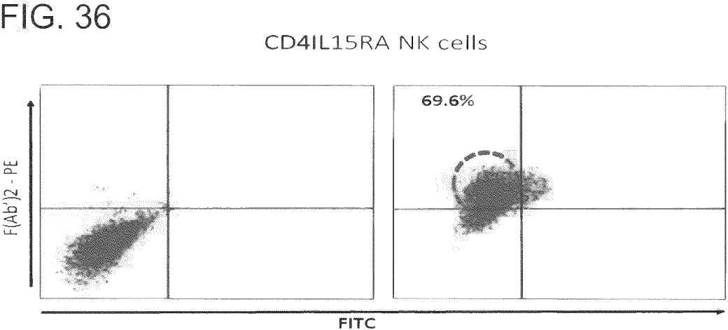

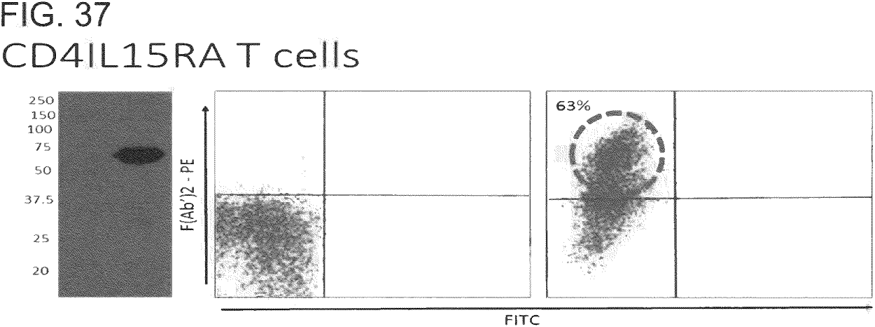

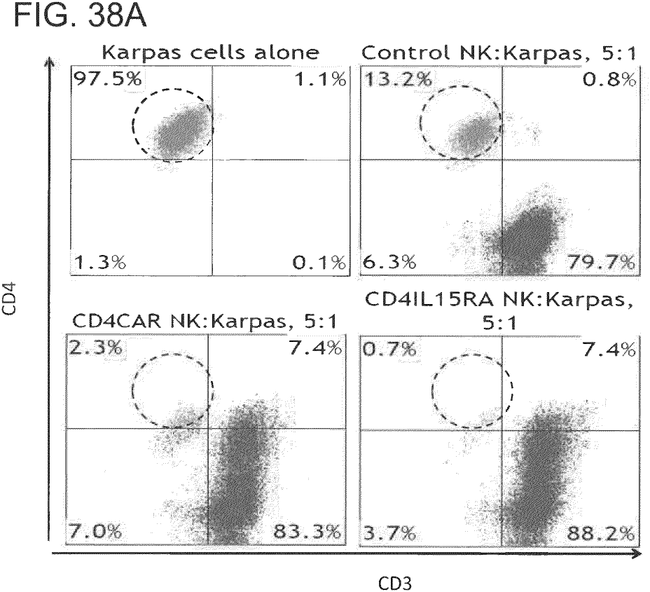

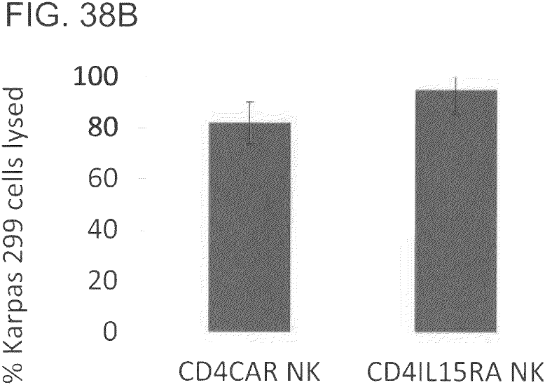

The Karpas 299 cell line was derived from a patient with anaplastic large T cell lymphoma. The MOLT4 cell line expressing CD4 was established from the peripheral blood of a 19-year-old patient with acute lymphoblastic leukemia (T-ALL). During 4-hour co-culture experiments, CD4IL15RA CAR NK cells showed profound killing (95%) of Karpas 299 cells at a 5:1 ratio of effector:target, at an even higher rate than that of CD4CAR NK cells (82%; FIG. 38). Similarly, when co-cultured 1:1 with MOLT4 cells, CD4IL15RA CAR NK cells lysed target cells at a higher rate (84% to 65%) than CD4CAR NK cells in an overnight assay (FIG. 39). These results show that CD4IL15 CAR NK cells can ablate tumor cells at least as well as CD4CAR NK cells.

### CD4CAR and CD4IL15RA CAR T Cells Exhibit More Potent Anti-Tumor Activity In Vivo Than CD4CAR

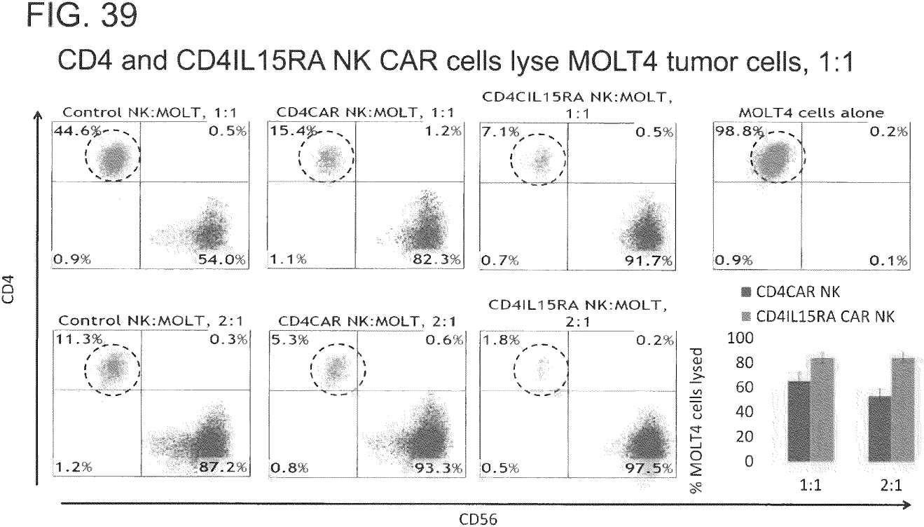

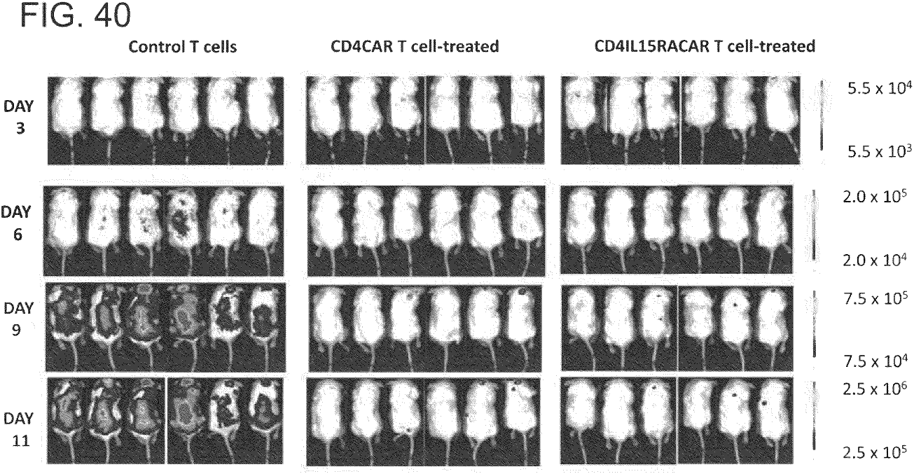

In order to evaluate the in vivo anti-tumor activity of CD4CAR and CD4IL15RACAR T cells, and to determine the possible increase in persistence of the CD4IL15RA CAR T cells relative to the CD4CAR T cells, we developed a xenogeneic mouse model using NSG mice sublethally irradiated and intravenously injected with luciferase-expressing MOLM13 cells, an acute myeloid leukemia cell line (M5) that is 100% CD4, to induce measurable tumor formation. Three days following tumor cell injection, 6 mice each were intravenously injected with a course of 8 × 10⁶ CD4CAR, CD4IL15RACAR T cells or vector control T cells. On days 3, 6, 9 and 11, mice were injected subcutaneously with RediJect D-Luciferin (Perkin Elmer) and subjected to IVIS imaging to measure tumor burden (FIG. 40). CD4CAR T cell-treated mice had a 52% lower tumor burden relative to control on Day 6, whereas CD4IL15RA CAR T cell-treated mice had a 74% lower tumor burden (FIG. 41). On Day 11, nearly all tumor cells had been lysed in both of these groups. Unpaired T test analysis revealed an very significant difference (P=0.0045) between control and the two groups by day 9 with less light intensity and thus less tumor burden in the CD4CAR and CD4IL15RACAR T cells treated group compared to control.

### Promoter Testing Using the GFP Reporter

HEK293FT cells were transfected with lentiviral plasmids expressing GFP under the SFFV, EF1 or CAG promoters. Approximately 60 hours after transfection, supernatant was collected from each. Relative viral titer was determined by first transducing HEK293 cells with supernatant from each of the 3 promoters. HEK-293 cells were transduced with GFP viral supernatant from each of the 3 transfected HEK-293FT cells. Polybrene was added to 4 uL/mL. Media was changed after 16 hours and replaced with media containing no viral supernatant or polybrene. Three days after transduction, cells were harvested and washed, suspended in 2% formalin, and analyzed by flow cytometry for GFP expression (FITC). GFP expression was seen in each sample, but was highest for the cells transduced with virus made using the SFFV promoter.

Activated human umbilical cord buffy coat cells were transduced with GFP lentiviral supernatant (amount based on the results of the HEK293 transduction efficiency) from each of the promoters. After 5 days incubation, cells were harvested, washed and suspended in 2% formalin, and analyzed by flow cytometry for GFP expression. 43% of cells expressed GFP at high levels (>10³) while GFP-expression for cells transduced with virus using promoters EF1 (15%) and CAG (3%) were considerably lower. Five days later, cells analyzed the same way showed nearly the same percentages for each (46%, 15% and 3%, respectively; FIG. 23). These results indicate that SFFV promoter leads to stronger expression than EF1 or CAG promoters, and that the expression remains high for at least 10 days post-transduction. Further experimental tests will include longer incubation times for transduced cells beyond the 10-day window.

Methods of generating the CAR gene including at least one of a T antigen recognition moiety (at least one of CD4, CD8, CD3, CD5, CD7, and CD2, or a part or a combination thereof), a hinge region and T-cell activation domains is provided.

Methods of generating multiple units of CARs (cCAR) targeting antigen (s) including at least one of CD33, CD123, CD19, CD20, CD22, CD269, CS1, CD38, CD52, ROR1, PSMA, CD138, and GPC3, or a part or a combination of a hinge region and T- cell activation domains is provided. All references cited and/or disclosed herein are hereby incorporated by reference in their entirety.

The provided methods also include: 1) generating of the CAR T or NK cells targeting leukemias and lymphomas expressing CD45 and avoiding self-killing; 2) generation of “armored” CAR T or NK cells designed to both overcome the inhibitory tumor microenvironment and exhibit enhanced anti-tumor activity and long-term persistence. The present invention is not limited to the embodiments described and exemplified above, but is capable of variation and modification within the scope of the appended claims. Various publications, including patents, published applications, technical articles and scholarly articles are cited throughout the specification. Each cited publication is incorporated by reference herein, in its entirety and for all purposes. Various terms relating to aspects of the invention are used throughout the specification and claims. Such terms are to be given their ordinary meaning in the art, unless otherwise indicated. Other specifically defined terms are to be construed in a manner consistent with the definition provided herein.

### Functional Titer of Viral Vector Particles in Supernatants (The % GFP Cells as Determined by Flow Cytometry Allows for Proxy Viral Titer Adjustments as Higher Titer Virus Infiltrates More Cells, Leading to Higher %GFP Cell Populations)

To determine functional titer of viral vector particles in each of our supernatants, HEK 293 cells were transduced with either EF1-GFP or SFFV-GFP viral supernatant, with either 30 µL (low), 125 µL (medium), or 500 µL (high) per well of a 12 well tissue-culture treated plate. Culture media was changed the following morning to DMEM plus 10% FBS.

Transduced cells were then trypsinized, washed, and suspended in formalin and subjected to flow analysis. The percentage of GFP+ cells in each of the conditions was determined by flow cytometry using the FITC channel (FIG. 43). In each case, the percentage of GFP+ was higher in cells transduced with SFFV-GFP than the cells transduced with the corresponding volume of EF1-GFP viral supernatant (50% to 18% for low, 80% to 40% for medium, and 82% to 70% for high). From this, we determined that using the highest volume of EF1-promoter virus was comparable to using the lowest volume of SFFV-promoter virus in terms of titer, and would allow for comparison of relative promoter strengths for the following transduction experiments

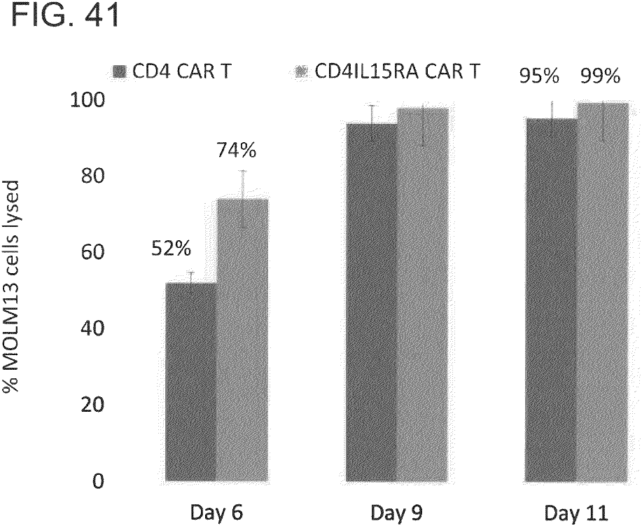

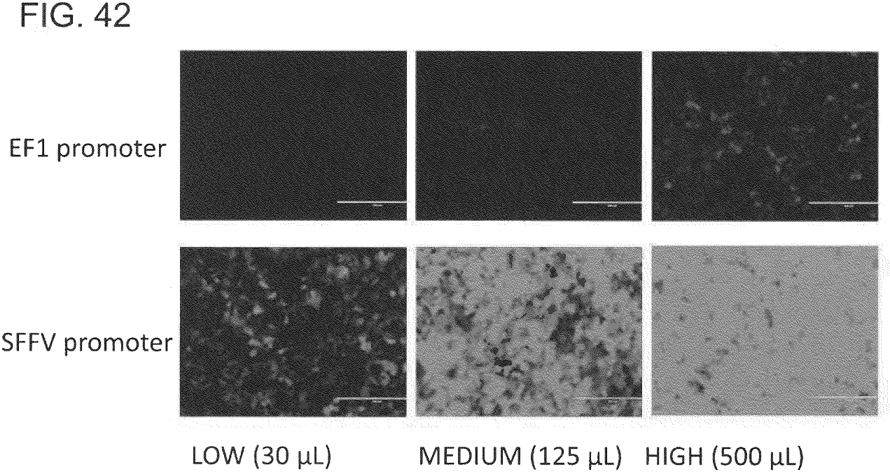

Transduced cells were also visualized on an EVOS fluorescent microscope using GFP at 20x at the same exposure conditions for each well (FIG. 42). Cells transduced with SFFV-GFP viral supernatant were dramatically brighter than cells transduced with EF1-GFP. Furthermore, comparing the image of the EF1-promoter under high viral volume loads with the image of the SFFV-promoter using low viral volume loads show similar fluorescent intensity. This suggests that the SFFV promoter is a stronger driver of gene expression.

### Comparison of Surface Expression and Persistence of Different Promoters in Primary T-Cells (The % GFP Cells as Determined by Flow Cytometry for T-Cell Transductions Show Expected Differences in GFP Cell Populations as Expected From the Prior Experiments on HEK293 Cells)

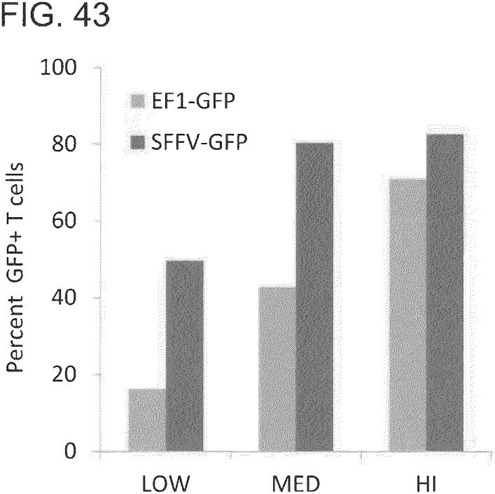

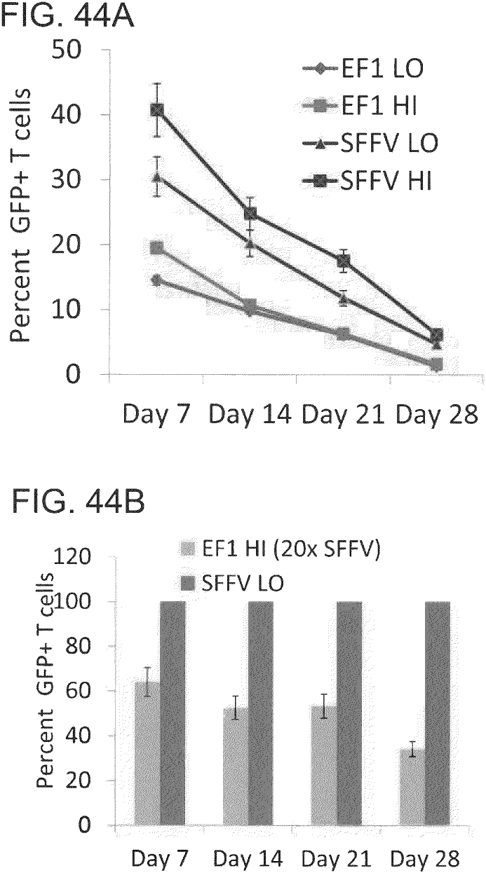

To determine promoter transduction efficiency and persistence of surface expression in primary T cells, activated cord blood buffy coat T cells were transduced with either 50 µL of SFFV-GFP or 1 mL of EF1-GFP EF1-GFP viral supernatant, in 12-well tissue culture-treated plates pre-coated with retronectin (Clontech). Following two overnight transductions, cells were cultured on T cell media with 300 IU/mL IL-2 (Peprotech) and maintained at 1.0-4.0 × 10⁶/mL. Cells were washed, suspended in formalin, and subjected to flow cytometry analysis, using the FITC channel to determine the percentage of GFP+ cells, on 7, 14, 21 and 28 days after transduction. The percentage of GFP+ cells was consistently higher for T cells transduced with SFFV-GFP compared to EF1-GFP-transduced T cells (FIG. 44A), even as the percentage of total GFP+ cells decreased over this period. A further comparison showed that T cells transduced with the higher (1 mL) amount of EF1-GFP supernatant actually decreased in percentage relative to the percent of GFP+ cells transduced with the lower amount (50 µL, or 20-fold less) of SFFV-GFP, between Day 7 and Day 28, from over 60% to under 40% (FIG. 44B). This suggests that transduction using the SFFV promoter led to greater persistence of transduced cells.

### BCMA or TACI or BAFF-R CAR NK Cells or T-Cells Targeting Cells Expressing at Least One of BCMA or TACI or BAFF-R CAR Antigen

To assess the cytotoxicity ability of CAR targeting at least one of BCMA or TACI or BAFF-R NK cells or T cells, co-culture assays are conducted with cell lines or primary human cells expressing at least one of BCMA or TACI or BAFF-R. The ability of the aforementioned CAR NK cells or T cells to lyse the target cells was quantified by flow cytometry analysis, and target cells were stained with Cytotracker dye (CMTMR). Lysis is observed at 24 hour long cultures.

### BAFF or APRIL CAR NK or T Cells Targeting Cells Expressing at Least One of BCMA or TACI or BAFF-R Antigen

The chimeric antigen receptor in the CAR is the ligand for BCMA or TACI or BAFF-R.

To assess the cytotoxicity ability of CAR targeting at least one of BCMA or TACI or BAFF-R NK or T cells, co-culture assays are conducted with cell lines or human primary cells expressing at least one of of BCMA or TACI or BAFF-R. The ability of the aforementioned CAR NK or T cells to lyse the target cells was quantified by flow cytometry analysis, and target cells were stained with Cytotracker dye (CMTMR). Lysis is observed at 24 hour-long cultures.

### Generating CD7 CAR T Cells Targeting CD7 Expressing Cells

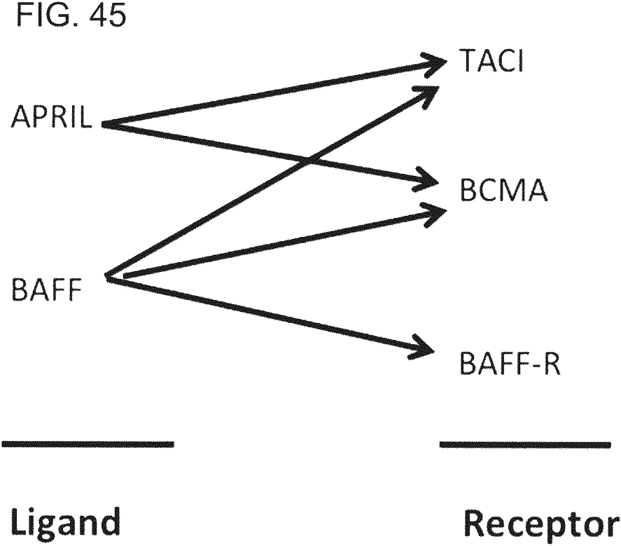

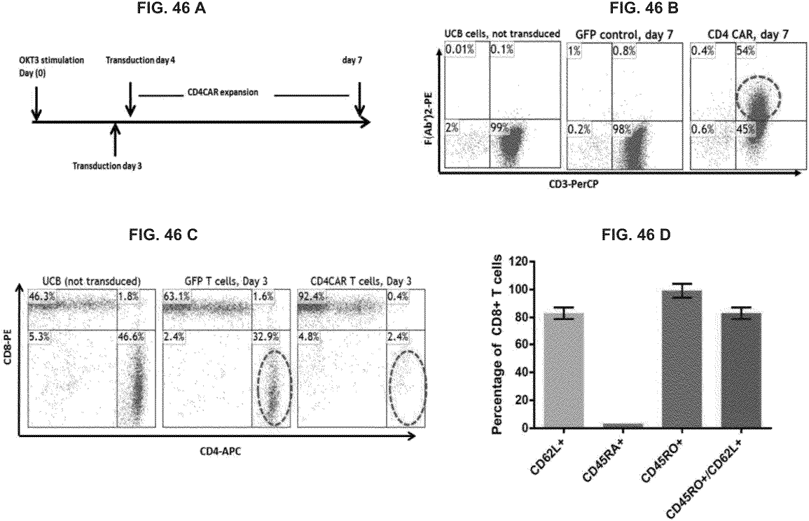

The organization of CD7 CAR (also called CD7-RTX CAR). CD7 CAR contains an anti-CD7 scFv, CD8 hinge and transmembrane regions, and a CD28 co-stimulatory domain fused to the CD3zeta signaling domain. The hinge region of CD7CAR also contains two RTX-binding epitopes. Expression is driven by the spleen focus-forming virus (SFFV) promoter (FIG. 46).

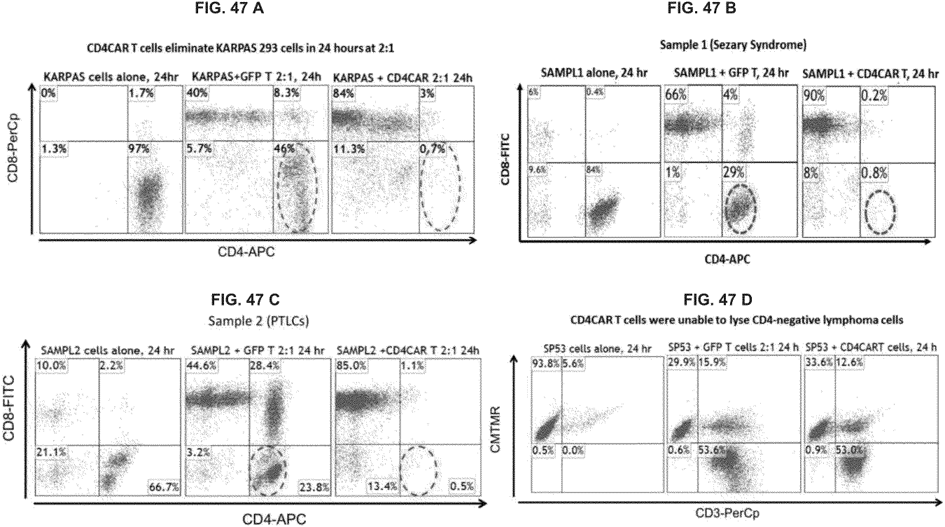

First, we confirmed the characteristics of CD7-RTX CAR. After transduction, flow cytometry analysis confirmed the expression of the CAR product, the availability of the rituximab-binding site, and the downregulation of CD7 in the transduced cells (FIG. 47). As the loss of CD7 may impair the proliferative capabilities of CD7CAR T-cells, we compared the growth of these cells to control cells and found that they expanded at a similar rate (FIG. 47), demonstrating that CD7 is not needed for T-cell proliferation. Depicted in (FIG. 47D). Characterization of CD7CAR and staining with goat-anti-mouse F(Ab′)2-Pe revealed a CAR expression of approximately ~70%. Staining with anti-human CD34 (used to detect the RTX-binding epitope) also demonstrated a transduction efficiency of ~80% (FIG. 2A and FIG. 2B). Staining with anti-human CD3 and anti-human CD7 demonstrate that the CD7CAR T-cells retain CD3 expression but lose expression of CD7 (FIG. 2C) likely due to the antibody-mediated internalization. CD7CAR T-cells can expand at rates similar to control T-cells despite losing CD7 expression (FIG. 47D).

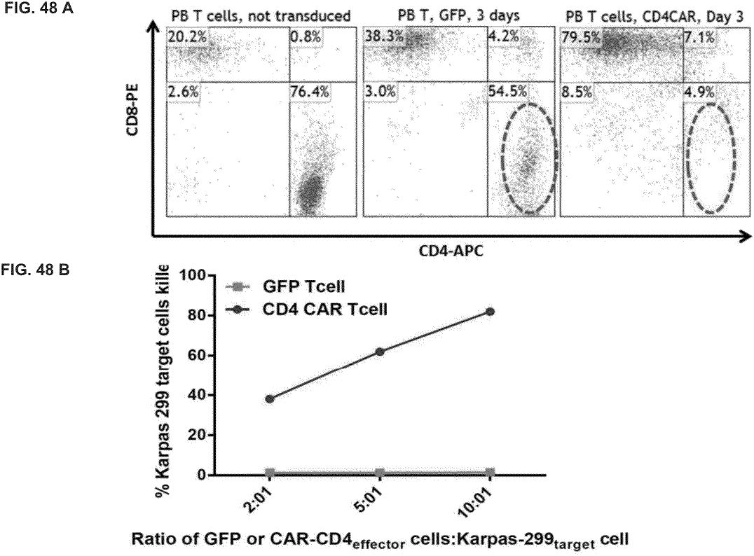

Next, second-generation CD7CAR T-cells derived from human umbilical cord blood that express CD28 and CD3zeta signaling moieties were co-cultured in vitro with the CCRF-CEM, Jurkat, and MOLT-4 cell lines, which consist of CD7+ T-ALL cells, and showed profound elimination of leukemic cells in an 18-hour incubation time (FIG. 48). CD7CAR demonstrates potent cytotoxicity against CD7+ cell lines in vitro. CEM-CCRF cells are ~90% CD7+ (bottom right panel). Control T-cells (left panels) or CD7-RTX T-cells (right panels) were placed with CEM-CCRF cells in an 18-h co-culture at E:T ratios of 1:1 (first row), or 2:1 (second row). Target CD7+ cells are circled in each panel. The results of the co-culture experiments demonstrate that CD7CAR T-cells lyse 99.88% and 99.82% of the CEM-CCRF cells relative to control at 1:1 and 2:1 respectively (FIG. 48).

In order to evaluate in vivo anti-tumor activities, CCRF-CEM leukemic cells were introduced into xenogeneic NSG mouse mice. Prior to injection, 12 mice were irradiated with a sub-lethal dose of gamma irradiation (2.0 Gy) and assigned randomly to the treatment or control group. Twenty-four hours later, mice were given one intravenous injection of 1.0×10⁶ CCRF-CEM cells.

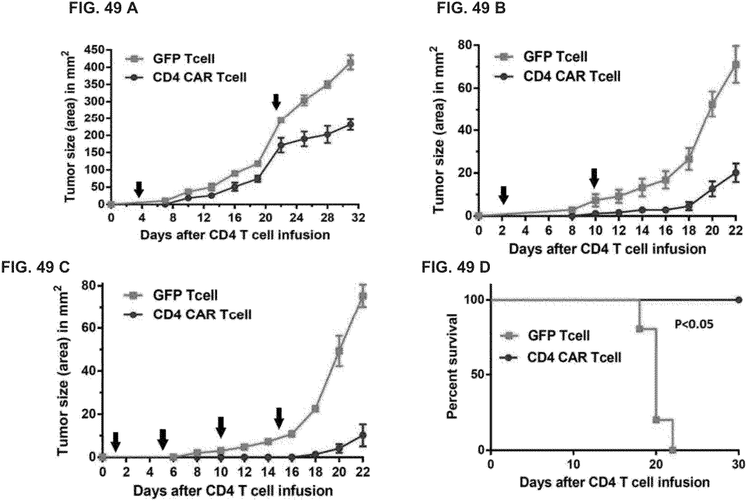

Five days following CCRF-CEM engraftment, mice were intravenously injected with 10×10⁶ CD7CAR or control T-cells. On days 5, 10, 13, 16, and 19, to evaluate tumor burden RediJect D-Luciferin (Perkin-Elmer) was injected intraperitoneally and mice were subjected to IVIS imaging to quantify luciferase activity. While flux, and thus tumor burden, continuously increased in control mice, it remained near background levels for the CD7CAR mice (FIG. 49). While control mice showed significant residual tumor population in the peripheral blood, CD7CAR-treated mice showed virtual depletion comparable to non-injected mice (FIG. 49). Additionally, CD7CAR-treated mice showed significant survival improvement (FIG. 49). CD7CAR improves outcome in in vivo model of T-ALL. NSG mice were sub-lethally irradiated and then intravenously injected on Day 1 with 1.0×10⁶ luciferase-expressing CEM-CCRF cells. Five days later, mice were injected with 10×10⁶ control or CD7CAR T-cells. Mice were injected with RediJect D-Luciferin on Days 5, 10, 13, 16, and 19 and subjected to IVIS imaging. Dorsal view. (FIGS. 47A-49A.) Total flux (photons/second) was measured and indicated a statistically significant difference in tumor burden between the two groups as early as day 8. The flux of the CD7CAR-treated mice had 41.3% (dorsal) less tumor by day 8, which increased to 99.6% (dorsal) by day 19 (FIGS. 47C-49C.). While all the control mice required euthanasia due to hindlimb paralysis and hunchback by day 24-26, the CD7CAR-treated mice survived significantly longer, remaining alive on Day 43 (FIGS. 47D-49D). Kaplan-Meier survival analysis curve (p = 0.0026).

The CD7 CAR engineered T cells were used to treat human patients having T-ALL with unexpected outcomes and results.

### Background:

A 31-year-old male presented with left neck swelling, a WBC (white blood count) of 352.27×10⁹/L, ETV6 mutation (40.8%), NOTCH1 mutation (41.8%), and NRAS mutation (44.6%), and chromosome fusion gene negative. The diagnosis of high-risk T-ALL was made, and the patient began multiple lines of chemotherapeutic treatment regimen followed by a sibling HSCT (human stem cell transplant). Despite a disease reduction from multiple chemotherapeutic treatments, including bone marrow transplant the patient suffered a disease recurrence at day 83. The patient was then enrolled for CD7 CAR T cell therapy.

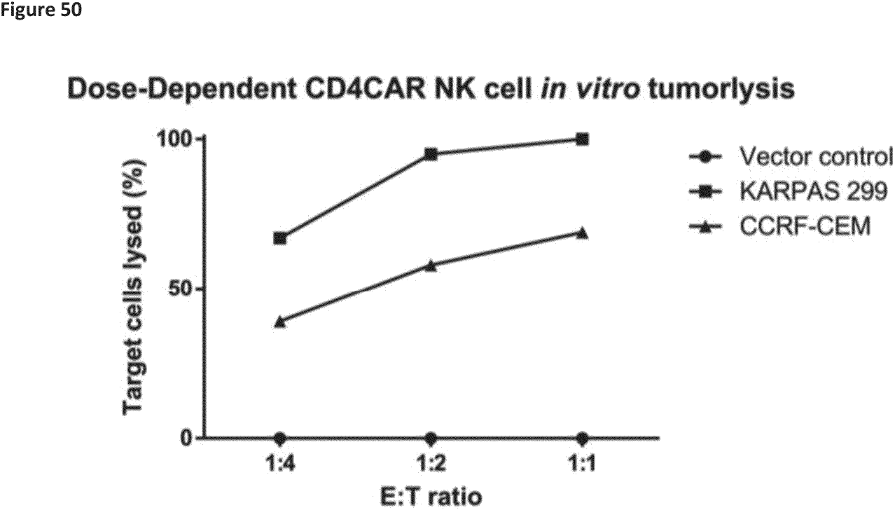

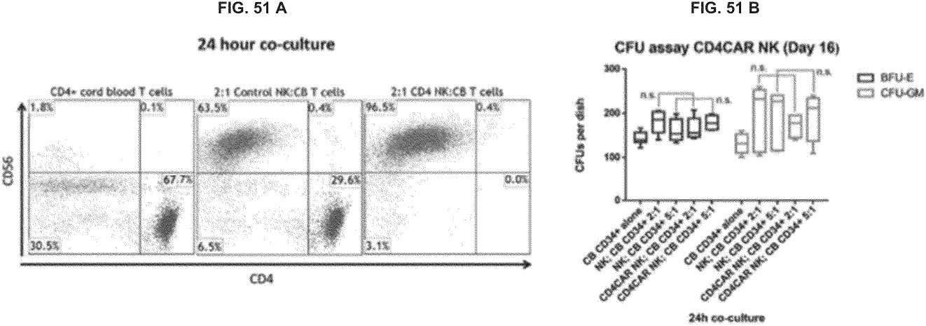

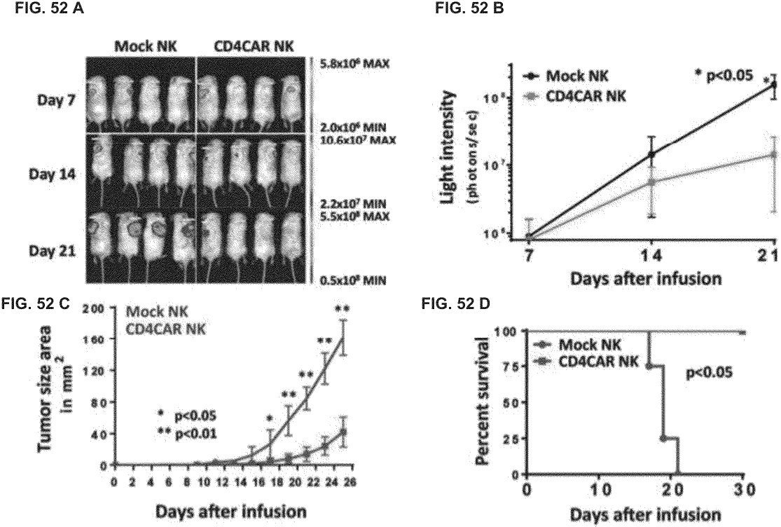

The patient’s relapsed T-ALL was positive for CD7 surface antigen, and thus was a candidate for CD7 CAR T cell therapy (FIG. 50). Donor lymphocytes was collected from the original donor who provided the bone marrow stem cells, were collected 6 days prior to CAR T treatment. The basal metrics prior to CAR-T treatment were as follows: blasts in marrow 24%, cerebral spinal fluid (CSF)1.77%, and chimerism 68.36%. A precondition treatment of Fludarabine and Cytarabine chemotherapeutics was administered 4 days prior to CD7 CAR T cell treatment. Anti-CD7-CAR T cells were administered on two occurrences, first (day 0) 1×10⁶/kg CAR T cells and second (day 2) 2×10⁶/kg CAR T cells. By day 7, the patient’s peripheral blood was negative for CD7+ T-ALL cells. Furthermore, day 11 indicated of CD7 positive T-ALL cells from peripheral blood as well as bone marrow were completely depleted by the CAR-T cell treatment and patient peripheral blood and bone marrow chimerism reached ~100%. As a result of CD7 CAR T cell treatment, the patient achieved rapid resolution of their leukemia, MRD negative and thus complete remission. Interestingly, virtual all CD7 positive T cells were eliminated day 11 post-CAR (FIG. 51). CD7CAR T cell treatment depleted all the CD7+ T cells, with a residual few percent of CD7 negative T cells able to replenish the absent T cell population to normal levels.

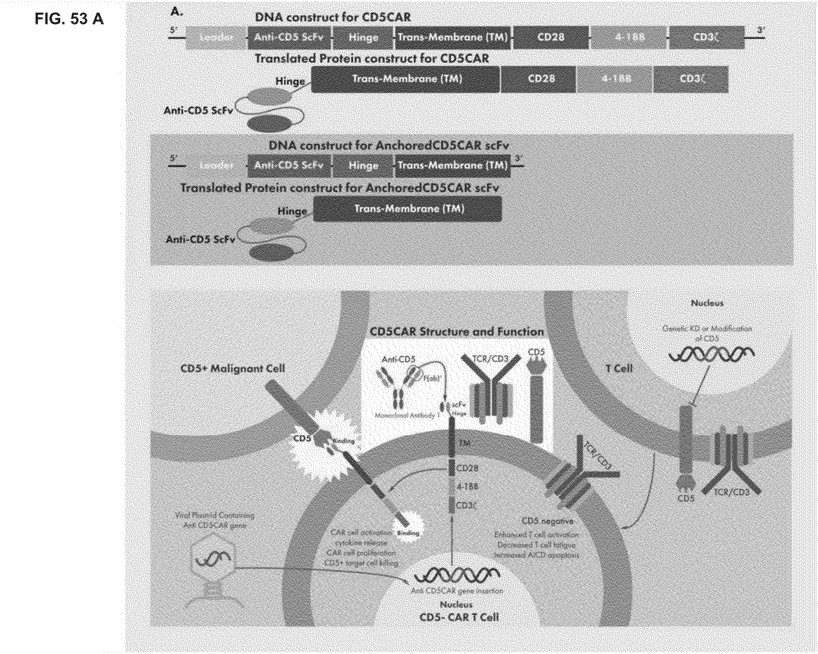

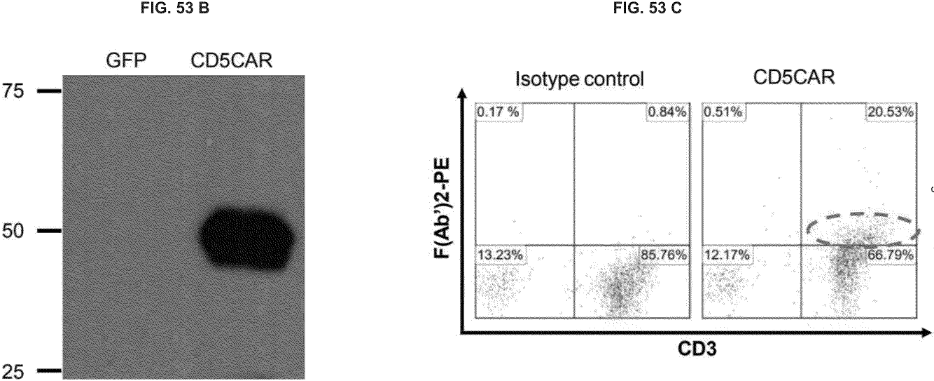

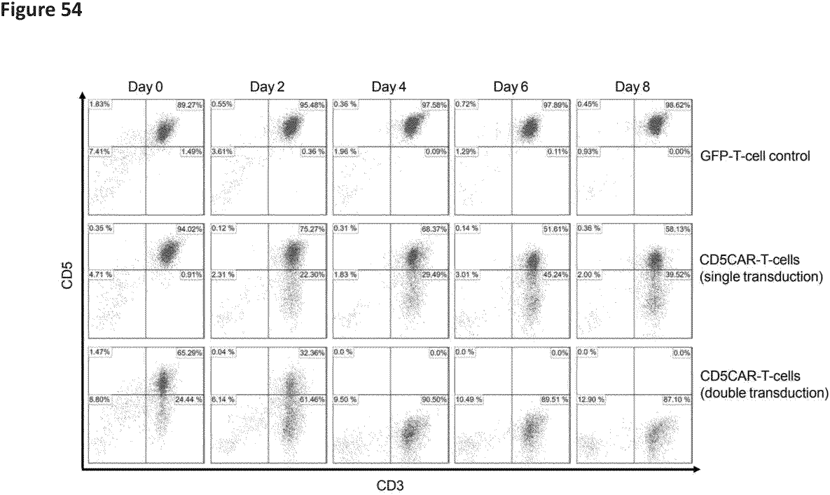

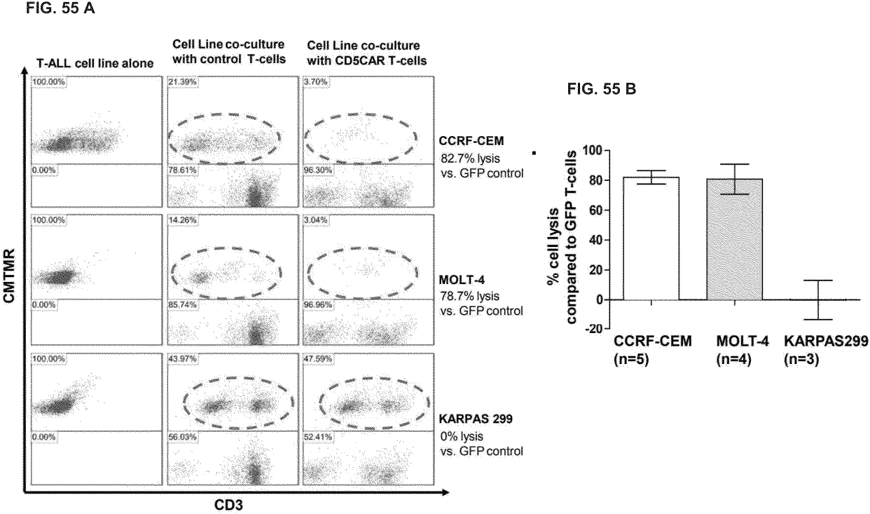

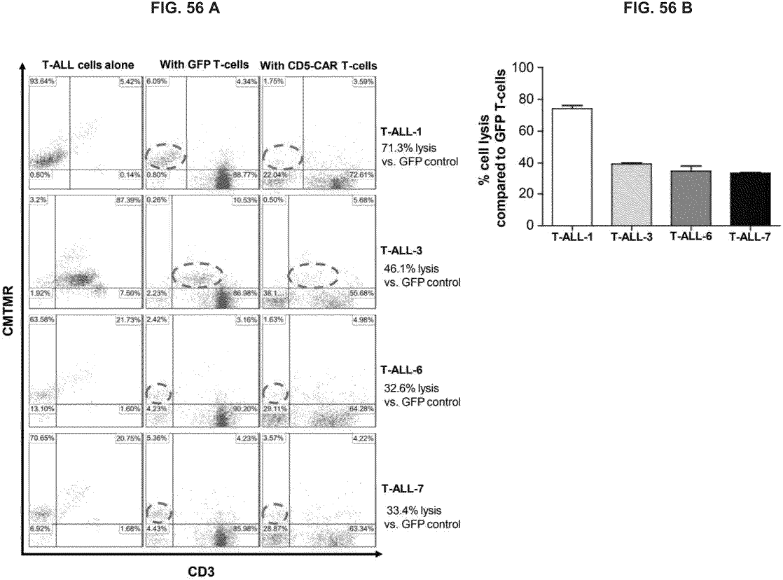

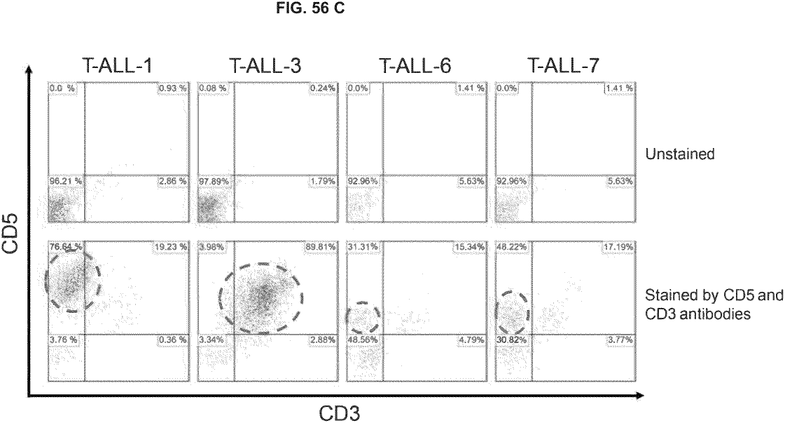

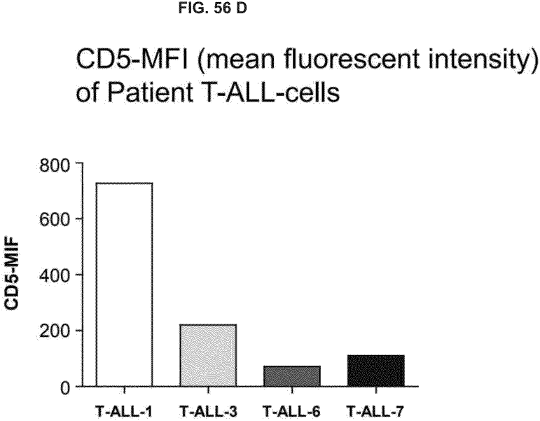

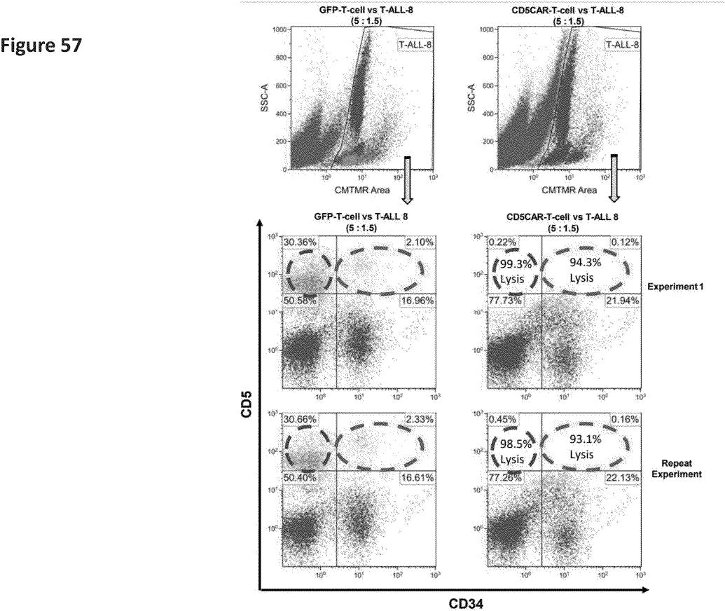

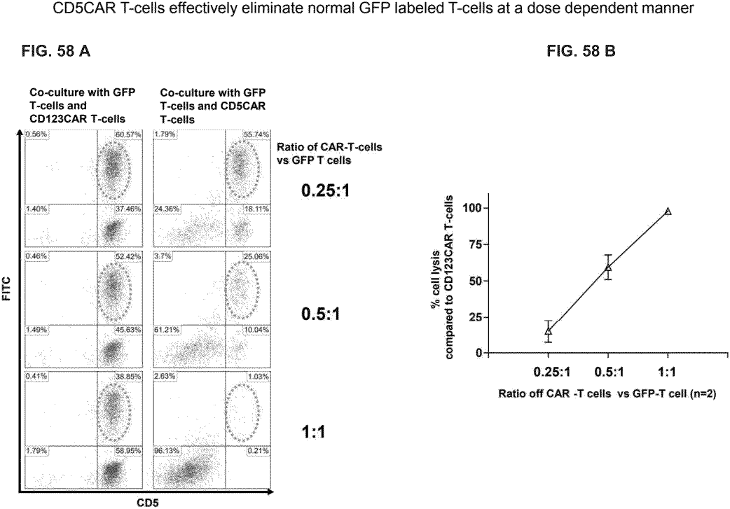

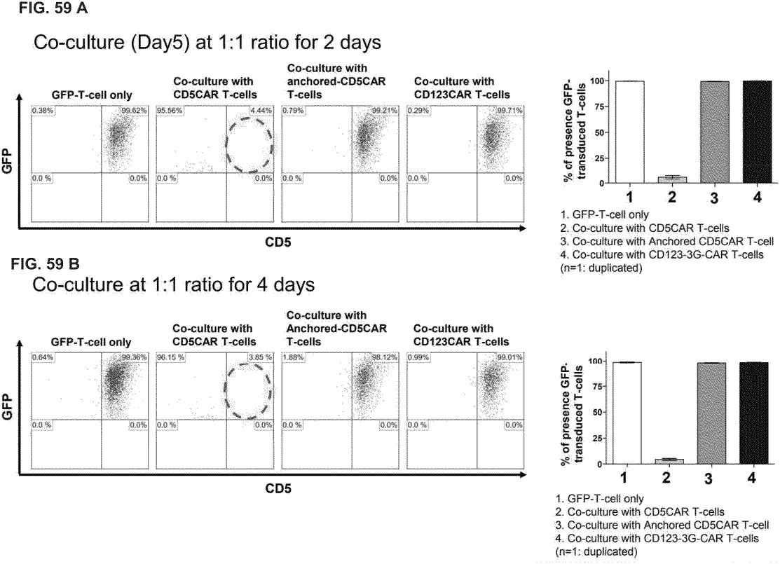

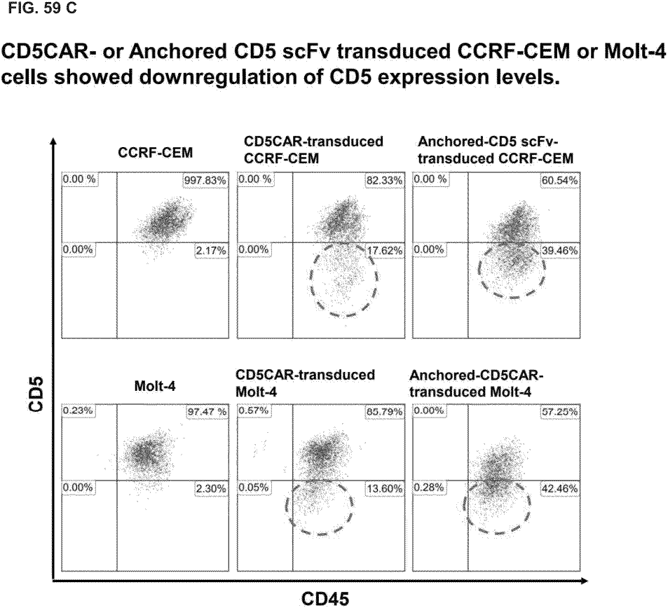

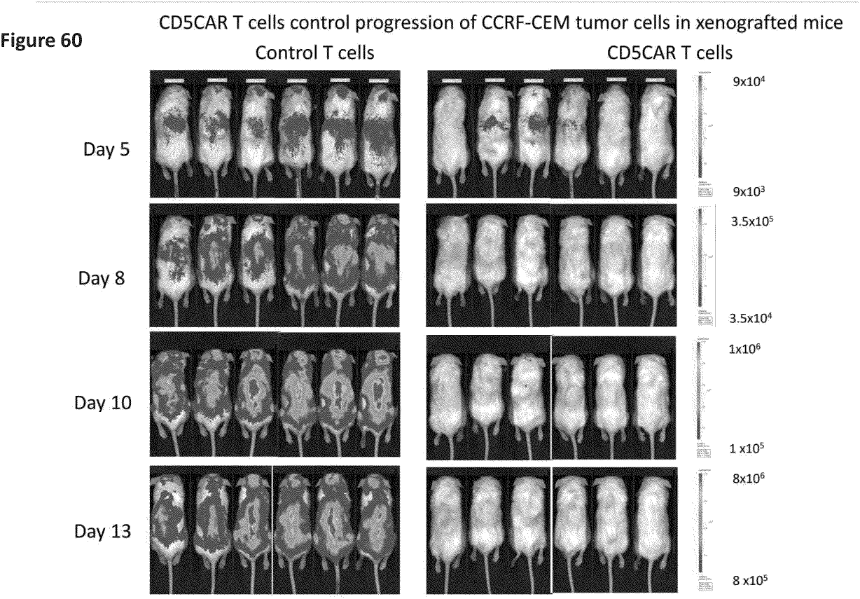

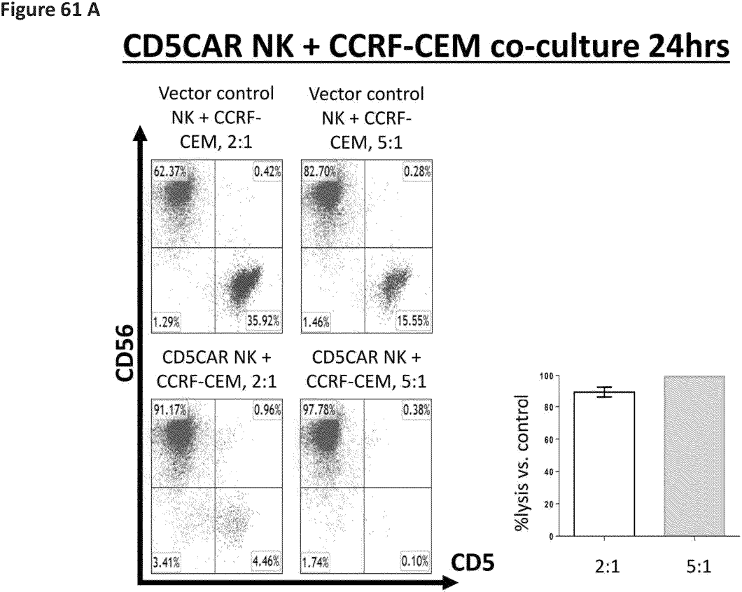

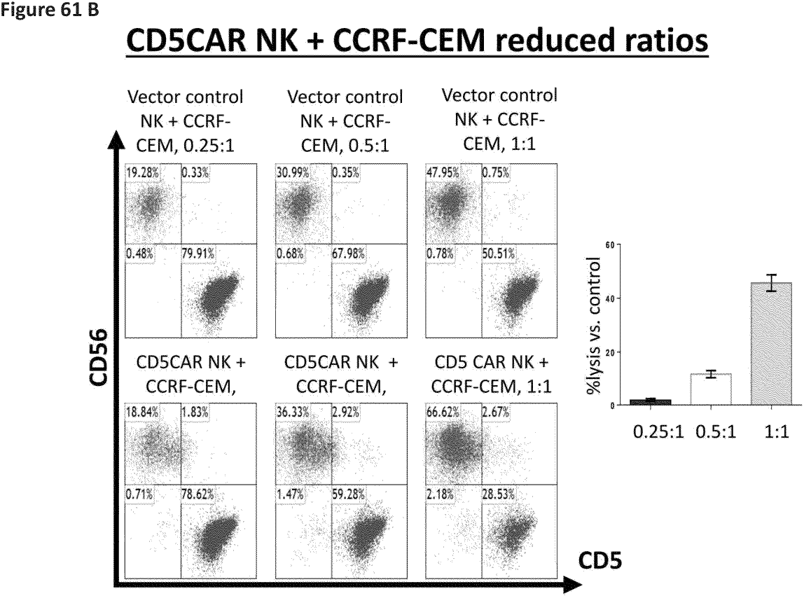

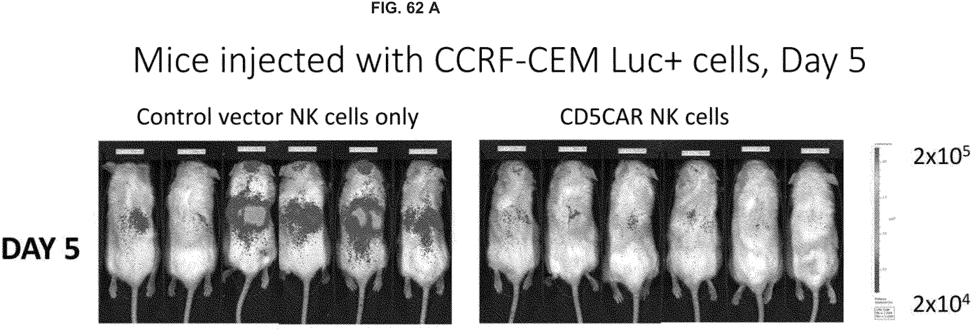

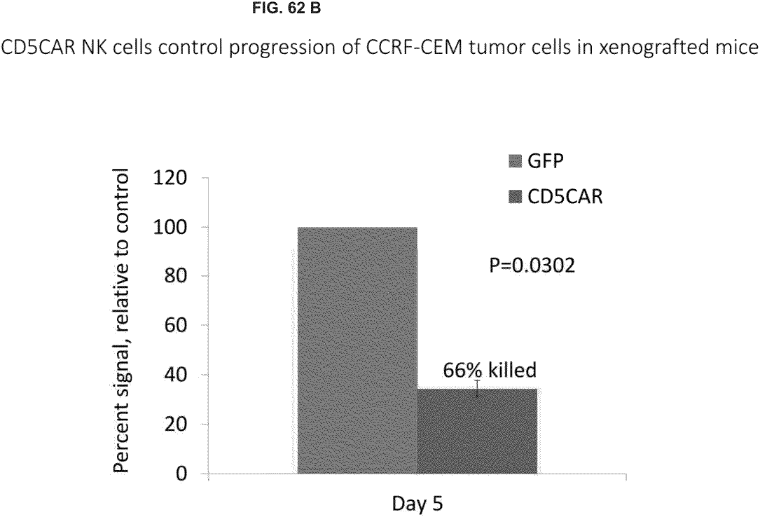

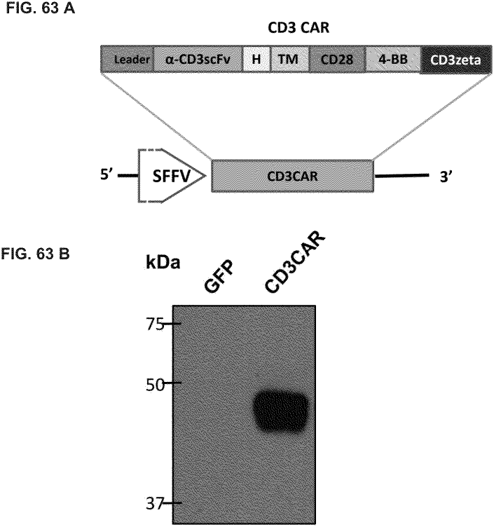

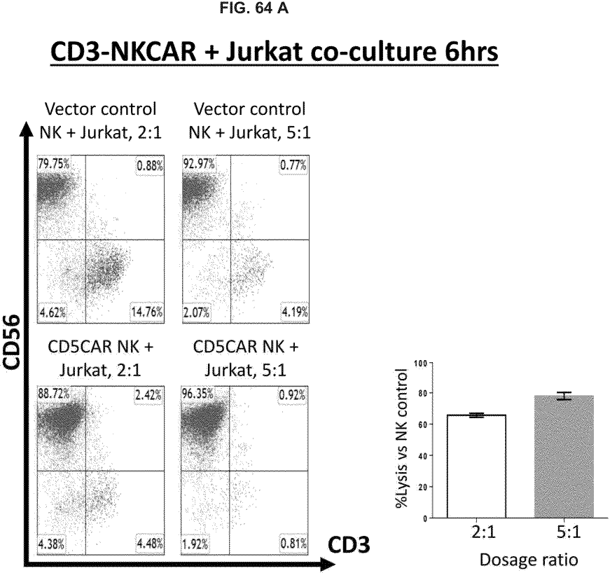

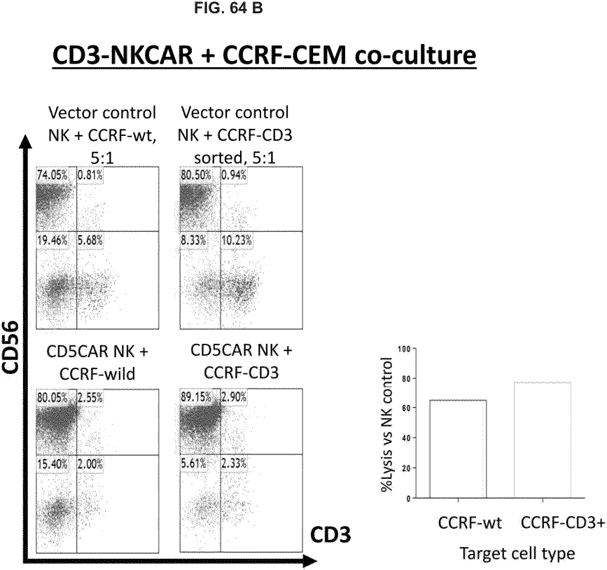

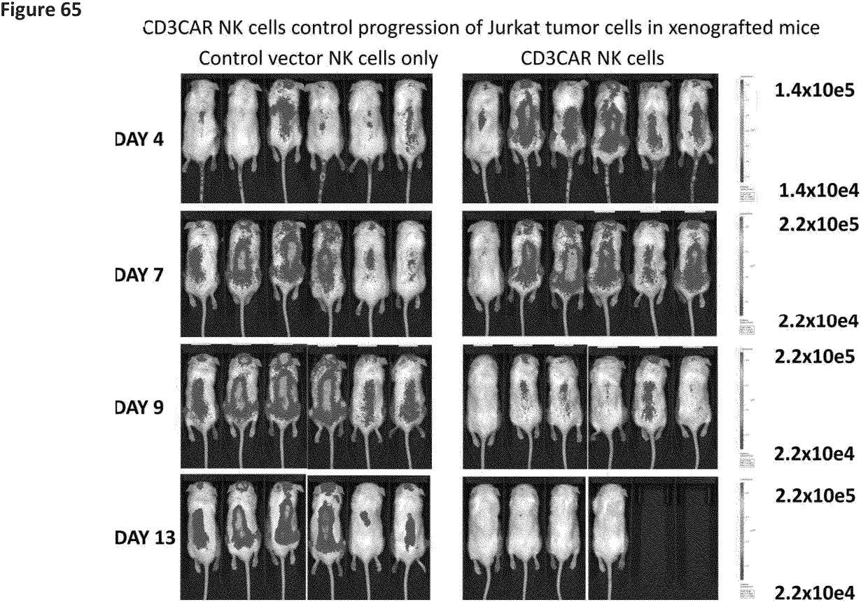

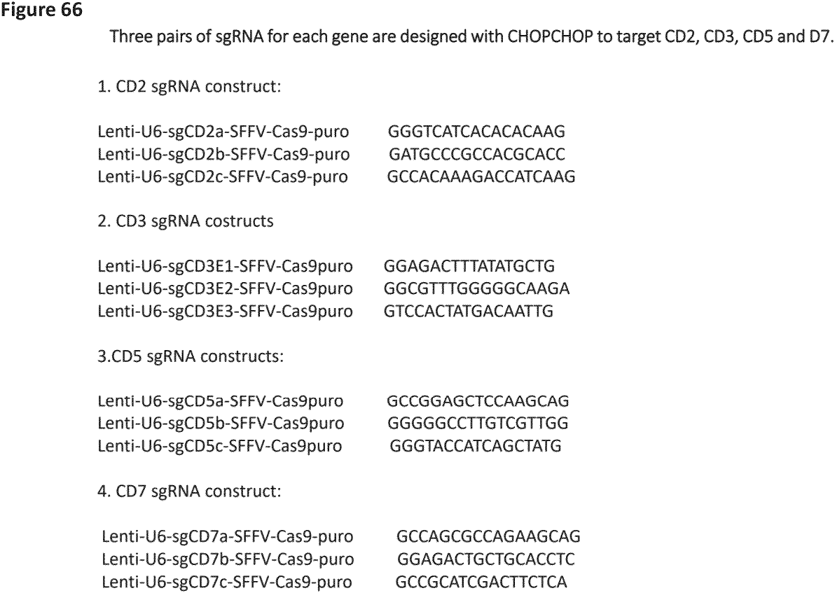

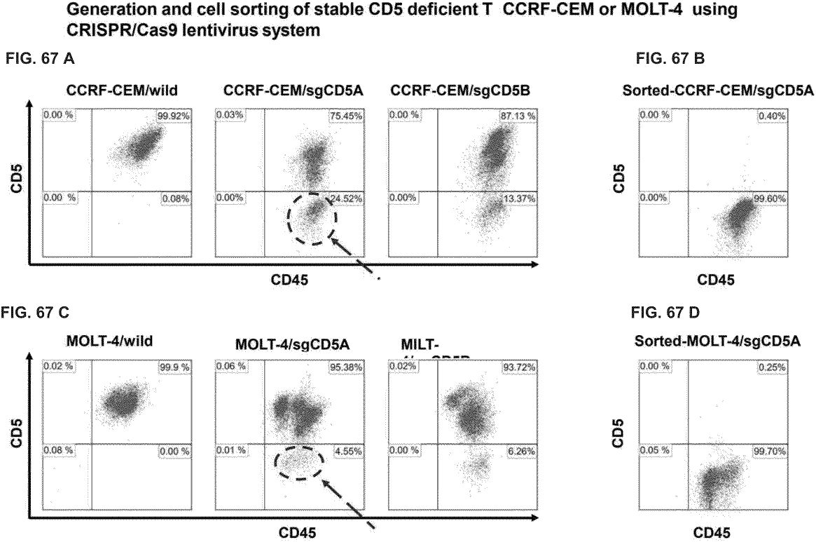

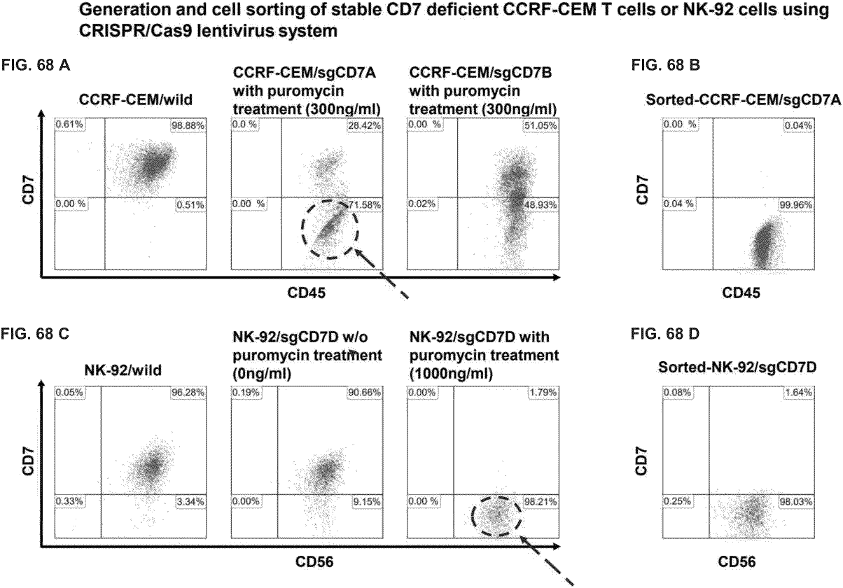

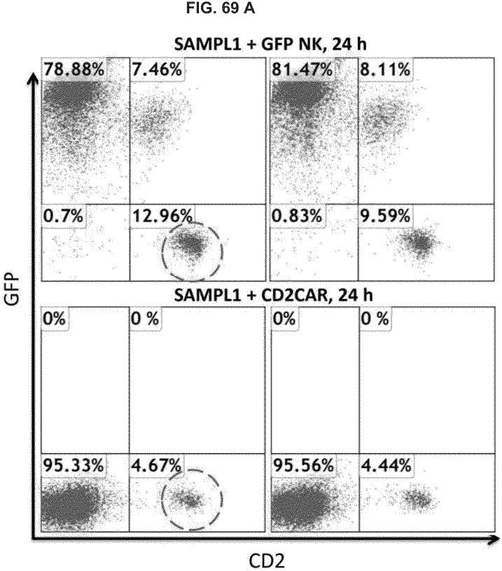

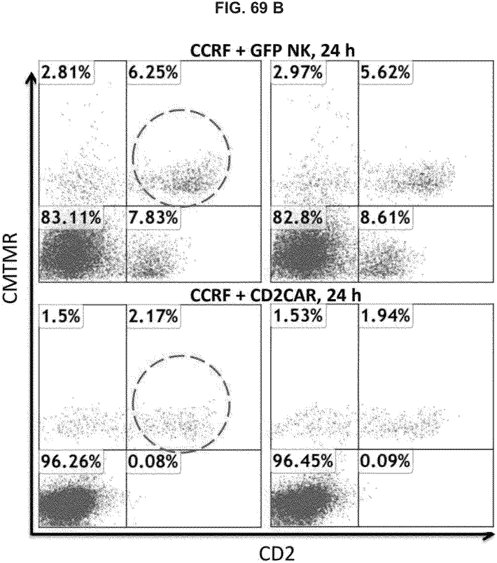

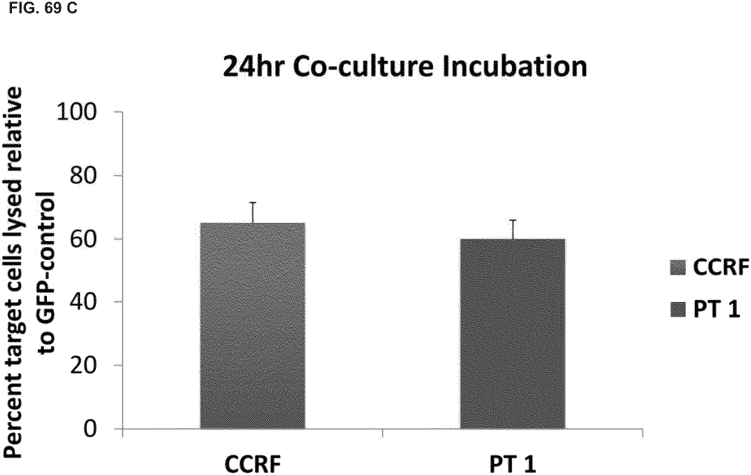

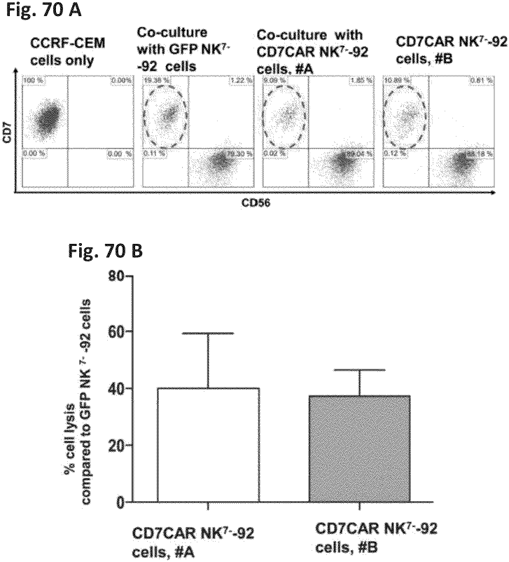

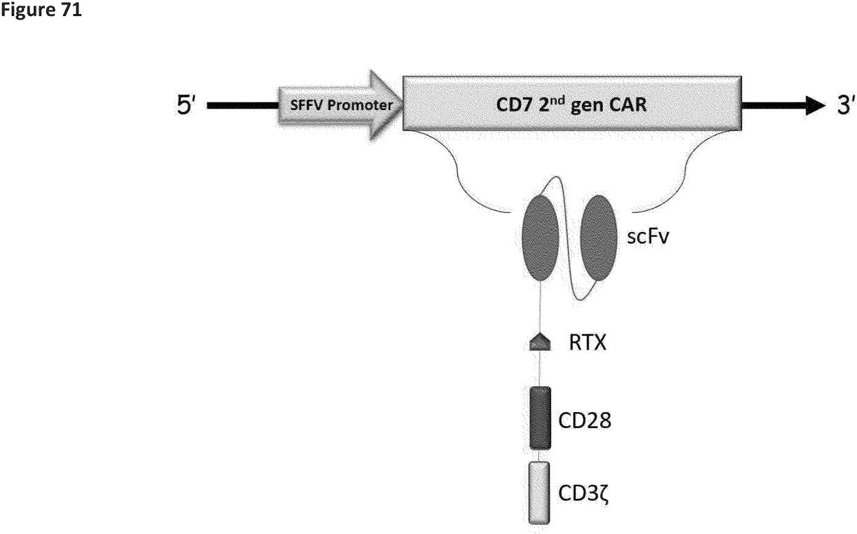

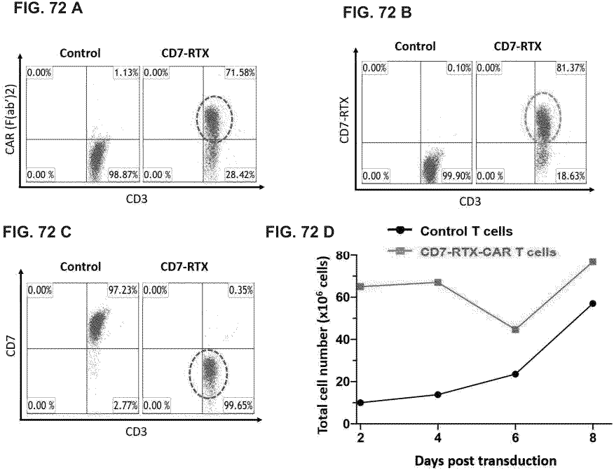

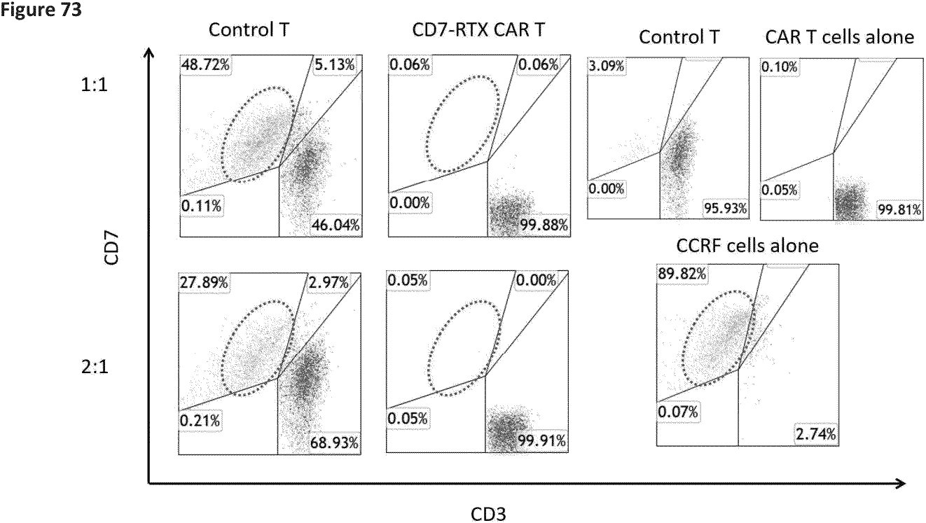

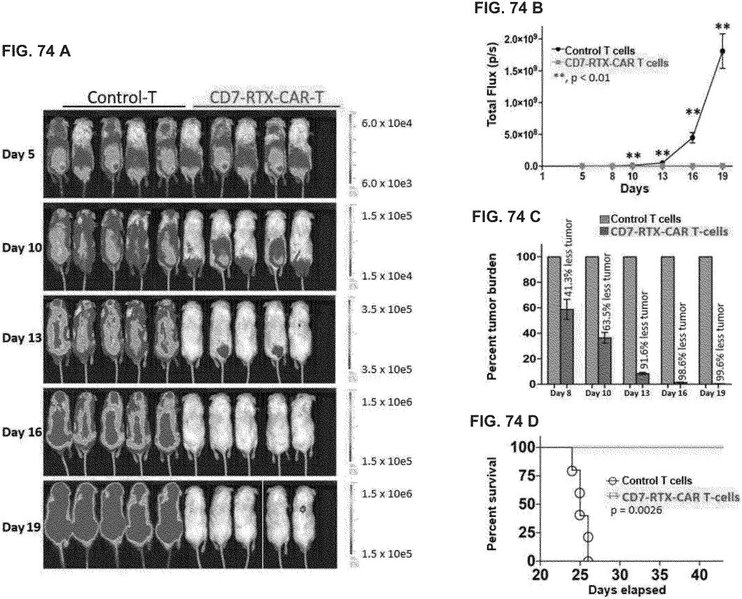

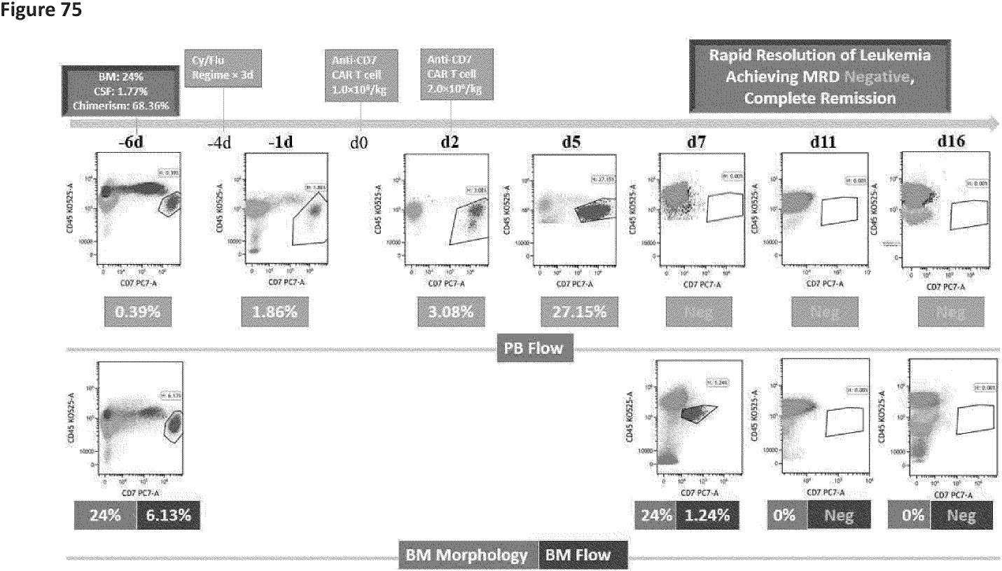

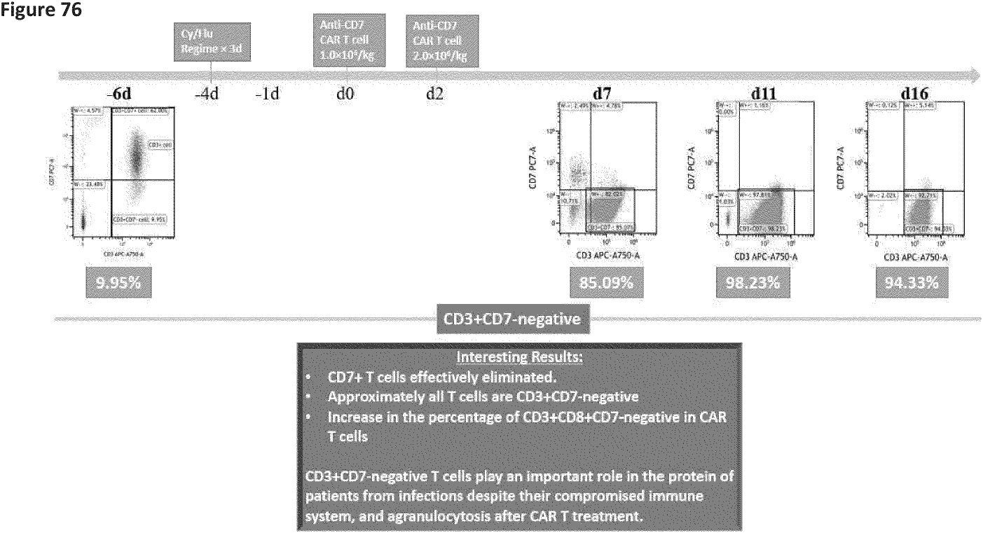

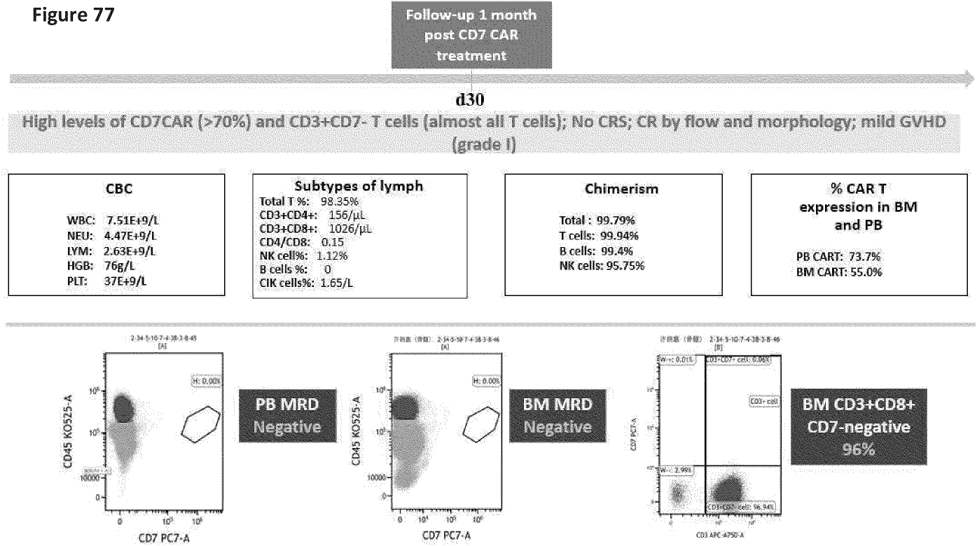

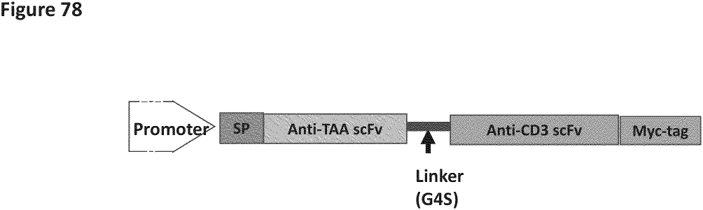

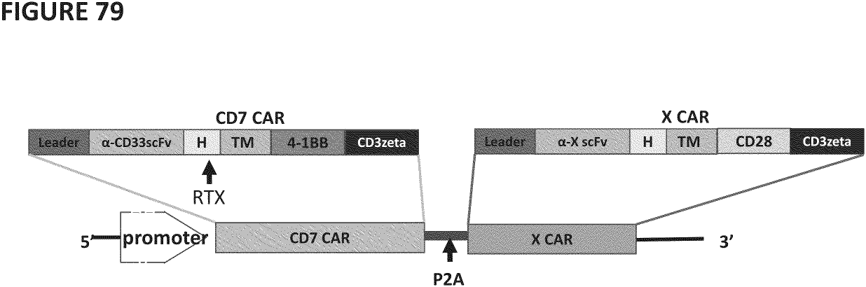

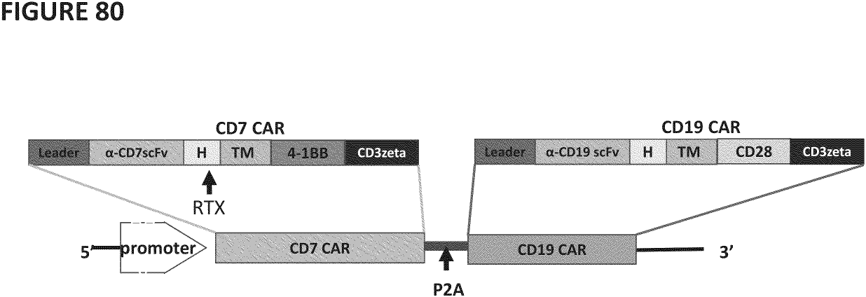

One-month post-CAR treatment (FIG. 53): The patient’s CBC indicated a WBC count of 7.51×10⁹/L, NEU 4.47×10⁹/L, LYM 2.63×10⁹/L, HGB 76 g/L and PTL 37×10⁹/L. Most noteworthy, the WBC and LYM count were restored to within the normal range respectively. The lymphocyte subtypes were as follows: total T 98.35%, absolute CD3+CD4+ 156/µL, CD3+CD8+ 1026/µL, Total T-cell 1182/µL (the total T cells are within the normal range). The chimerism was as follows: Total 99.79%, T cells 99.94%, B cells 99.4%, and NK cells 95.75%. In summary, at the patients one-month post CD7 CAR T cell treatment follow- up, high levels of CD7 CAR (>70% detected) and all T are absent of surface protein CD7. The CD7 CAR T cell treatment depleted all of the leukemia cells as well as all T cells expressing CD7 surface protein. The treatment was both efficacious and safe with the patient sustaining treatment with grade 1 cytokine release syndrome (CRS).

In one exemplary embodiment of the invention, T cells expressing CD7CAR were discovered to be effective in treating a human patient suffering from T cell acute lymphoblastic leukemia (T-ALL) expressing CD7. It is known that about 98% of a human’s T cell population are CD7 positive. Therefore, it was thought that administering a CD7CAR would deplete a human’s T cells and result in death. We unexpectedly discovered that the about 2% of T cells negative for CD7 surface antigen can rapidly expand and replace the eliminated CD7 positive T cell population to a relative normal range within a short time. As a result, a human receiving the CD7CAR tolerates the treatment. At the time of filing, creating CD7CAR T cells according to the claimed invention was against reason because it was thought that the CD7 antigen plays an important role in T cell based killing mechanisms, and elimination of CD7 positive T cells would have fatal results for the patient. Surprisingly, we discovered that the CAR T cell surface protein remained potent in its killing affects, despite the absence of CD7 surface protein, thus elucidating an unexpected finding. In this particular embodiment, the CD7CAR T cells were successful in treating patients with a high burden disease of T cell acute lymphoblastic leukemia.

Summarily, prior to the state of the art and teachings, a person of ordinary skill would not create a CD7CAR because the art teaches targeting of CD7 antigen results in total T cell deficiency or depletion, which can be associated with on-target toxicity of severe infections and is incompatible with a patient’s life. It is known that about 95% of a human’s T cell population are CD7 positive. We unexpectedly discovered that the about 2-5% of T cells negative for CD7 surface antigen can rapidly expand and replace the eliminated CD7 positive T cell population to a relative normal range within a short time. As a result, a human receiving the CD7CAR tolerates the treatment.

I was surprised to observe the following: 1) CD7CAR can be used in treating relapsed/refractory T-ALL patients with remarkable outcomes; 2) CD7 CAR T cell treatment depleted all the CD7+ T cells, with a residual few percent of CD7 negative T cells replenished the absent T cell population to normal levels; and 3) while CD7 CAR T cells without the surface CD7 expression in the patient demonstrate remarkable efficacy of depletion of CD7+ leukemic cells in the peripheral leukemia cells, the T cell level in the peripheral blood is near normal.

In one embodiment, a CD7 CAR targeting CD7 surface antigen can deplete the autoreactive immune cell expressing the CD7 surface antigen. The CD7+ population will be depleted (approximately >90 T lymphocytes) and an unexpected finding was elucidated in that the CD7- population of T lymphocytes expand to maintain the total T-cell population and prevent infection. Such an occurrence acts as an immune system reset for the T-cell immune system thus treating T-cell mediated autoimmune disorders.

In one embodiment, the engineered cell with CD7-RTX CAR includes a CD7 chimeric antigen receptor polypeptide and two CD20 binding epitopes (also called RTX-binding epitopes) in the hinge region (SEQ ID NO. 44), and corresponding nucleotides (SEQ ID NO. 45).

In one embodiment, the engineered cell with CD7-RTX VAC CAR (also called CD7-RTX-IL-15/IL15sushi CAR) includes a CD7 chimeric antigen receptor polypeptide, two CD20 binding epitopes (also called RTX-binding epitopes) in the hinge region, secreting IL-15/IL15sushi (SEQ ID NO. 46), and corresponding nucleotides (SEQ ID NO. 47).

In one embodiment, the engineered cell with CD7-RTX-CD19 cCAR includes a CD7 chimeric antigen receptor peptide bearing two CD20 epitopes in the hinge region, a CD19 chimeric antigen receptor polypeptide (SEQ ID NO. 48), and corresponding nucleotides (SEQ ID NO. 49).

In one embodiment, the engineered cell with CD7-RTX-CD19 VAC cCAR includes a CD7 chimeric antigen receptor peptide bearing two CD20 epitopes in the hinge region, a CD19 chimeric antigen receptor polypeptide, secreting IL-15/IL-15sushi (SEQ ID NO. 7), and corresponding nucleotides (SEQ ID NO. 8).

In one embodiment, the engineered cell with CD7-RTX-CD20h cCAR (CD7-RTX-CD20 cCAR) includes a CD7 chimeric antigen receptor peptide bearing two CD20 epitopes in the hinge region, a CD20 chimeric antigen receptor polypeptide (SEQ ID NO. 50), and corresponding nucleotides (SEQ ID NO. 51).

In one embodiment, the engineered cell with CD7-RTX-CD20 VAC cCAR includes a CD7 chimeric antigen receptor peptide bearing two CD20 epitopes in the hinge region, a CD20 chimeric antigen receptor polypeptide, secreting IL-15/IL-15sushi (SEQ ID NO. 52), and corresponding nucleotides (SEQ ID NO. 53).

In one embodiment, the engineered cell with CD7-RTX-CD33 VAC cCAR includes a CD7 chimeric antigen receptor peptide bearing two CD20 epitopes in the hinge region, a CD33 chimeric antigen receptor polypeptide, secreting IL-15/IL-15sushi (SEQ ID NO. 54), and corresponding nucleotides (SEQ ID NO. 55).

In one embodiment, the engineered cell with CD7-RTX-CLL1 VAC cCAR includes a CD7 chimeric antigen receptor peptide bearing two CD20 epitopes in the hinge region, a CLL1 chimeric antigen receptor polypeptide, secreting IL-15/IL-15sushi (SEQ ID NO. 56), and corresponding nucleotides (SEQ ID NO. 57).

In one embodiment, the engineered cell with CD7-RTX-CS 1 cCAR includes a CD7 chimeric antigen receptor peptide bearing two CD20 epitopes in the hinge region, a CS 1 chimeric antigen receptor polypeptide(SEQ ID NO. 58), and corresponding nucleotides (SEQ ID NO. 59).

In one embodiment, the engineered cell with CD7-RTX-BCMA cCAR includes a CD7 chimeric antigen receptor peptide bearing two CD20 epitopes in the hinge region, a BCMA chimeric antigen receptor polypeptide (SEQ ID NO. 60), and corresponding nucleotides (SEQ ID NO. 61).

In one embodiment, the engineered cell with CD7-RTX-BCMA VAC cCAR includes a CD7 chimeric antigen receptor peptide bearing two CD20 epitopes in the hinge region, a BCMA chimeric antigen receptor polypeptide, secreting IL-15/IL-15sushi (SEQ ID NO. 62), and corresponding nucleotides (SEQ ID NO. 63).

In one embodiment, the engineered cell with CD7-RTX-CD38 cCAR (CD7-RTX-CD38a cCAR) includes a CD7 chimeric antigen receptor peptide bearing two CD20 epitopes in the hinge region, a CD38 chimeric antigen receptor polypeptide (SEQ ID NO. 64), and corresponding nucleotides (SEQ ID NO. 65).

### Resolution of CAR-CAR Interaction and Increased Insert Size by a Compound CAR-BiTE Associated With Reduction of the Viral Titer

In one embodiment, two or more units of CARs expressing in a cell need to avoid CAR-CAR interaction.

In one embodiment, the hinge region is designed to exclude amino acids that may cause undesired intra- or intermolecular interactions. For example, the hinge region may be designed to exclude or minimize cysteine residues to prevent formation of disulfide bonds. In another embodiment, the hinge region may be designed to exclude or minimize hydrophobic residues to prevent unwanted hydrophobic interactions.”

in another embodiment, the transmembrane domain is selected or modified by amino acid substitution to avoid binding of such domains to the transmembrane domains of the same or different surface membrane proteins to minimize interactions with other members of the receptor complex.

The novel compound CAR contains a set of two-CAR polypeptides with 12 functional domains along with a self-cleavage sequence which is greater than the single CAR which contains 6 functional domains. As discussed in the previous office actions, creating a compound CAR according to the claimed invention was against reason at the time of filing because it was thought that the longer the polypeptide, the less effective it becomes for making viruses for CARs and insufficient killing. This was due to a lower level of protein expression expected with increasing size. Research has shown the level of expression drops with increasing insert size (Int J Biochem Mol Biol. 2013; 4(4): 201-208). Therefore, a person of ordinary skill in the art would not motivate to use to generate a large sized compound CAR or CAR with BiTE.

During a clinical trial with a compound CAR, it was surprisingly discovered that CAR T cells with CAR efficiencies below or around 5% can still eliminate targeted cells, leukemic cells and result in completion remission. This suggests that alternate/weaker promoters can be used in making effective cCAR T cells in accordance with the claimed invention.

### Inventive Steps by Surprising Discovery to Overcome the Low Titer of Viruses With a Large Sized cCAR or cCAR -BiTE

It was surprisingly finding that retroviral stable producer cell lines could resolute this problem related to the low titer of viruses with a large sized CAR.

In particular embodiments, generation of a high titer of cCAR or cCAR-BiTE can be achieved by combination of at least one or more of the following steps:

1. Lenti or retroviral cCAR or cCAR-BiTE is transduced to a stable producer cell line wherein non-cytotoxic viral envelops, such as RD114 is substituted.

2. The CAR expression in the stable producer cell line is determined by flow cytometry assay with anti-Fab antibodies

3. Sorting the stable producer cells with high expression of cCAR or cCAR-BitTE using fluorescence activated cell sorting (FACS) with anti-FAB antibodies

4. Expansion of sorted cells with a high level of CAR expression is performed and then test their secreted retroviruses or lentiviruses.

5. Cloning a highly expressed cell by limiting dilution.

### Example for Generation of a High Titer of Large Sized BCMA-CD19 VAC cCAR Retroviruses Using a Stable Producer Cell Line

BCMA-CD19 VAC cCAR (also called BCMA-CD19 IL-15/IL-15sushi cCAR) is a two unit CAR composed of a complete BCMA-CAR fused to a complete CD19 CAR by a self-cleaving, enabling independent expression of both CAR receptors separately on the T cell surface. Each CAR contains a CD8 leader, a CD8-derived hinge (H) and transmembrane (TM) regions, and co-activation domains linked to the CD3ζ signaling domain. An enhancer, IL-15/IL-15sushi is also separated from the first CAR and second CAR by a second cleavage site at the C-terminus.

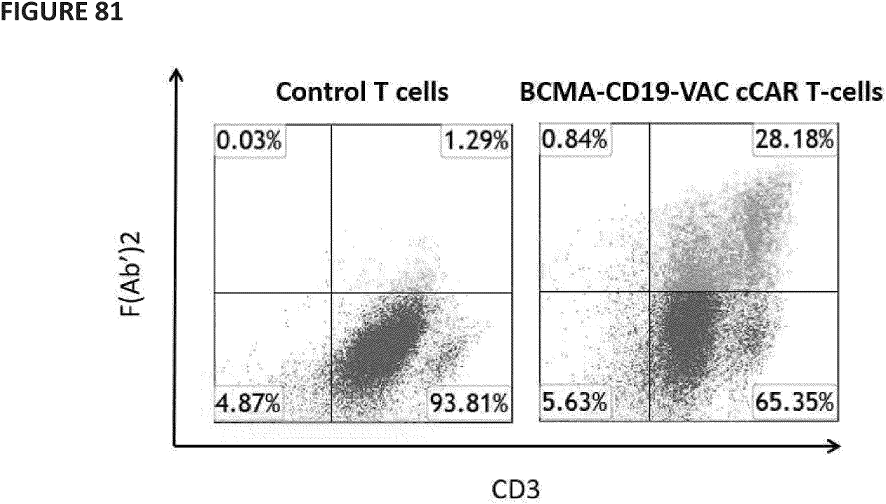

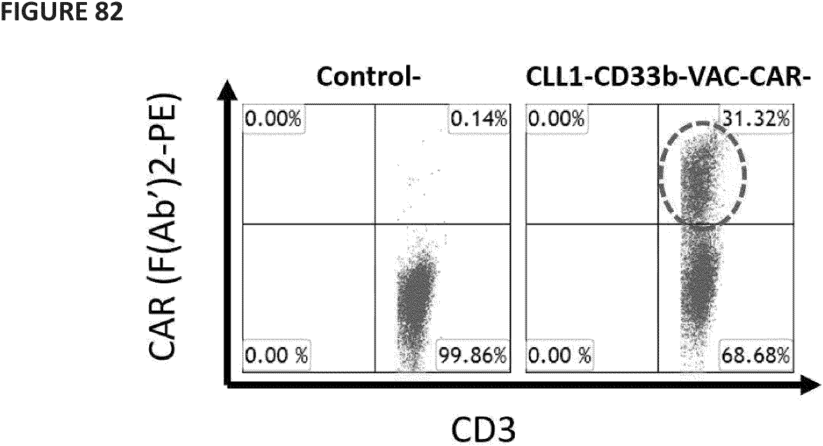

The BCMA-CD19 VAC cCAR contract was transfected into the H29 cell line (transient), viruses from transduced H29 were then transduce on the RD114 cell line (293, envelope protein from RD114 retrovirus). This envelope protein is utilized because it is not toxic to the cell. Cell sorting was used to identify highly expressing CAR population. The sorted population of highly expressing CAR cells was then expanded. The viruses secreted from expanded cells were then tested on T-cells for CAR expression. Additionally, a single clone that expresses a high level of CAR was cloned by limiting dilution. Viruses from a highly expressed clone was tested for CAR expression by flow cytometry. Flow cytometry results showed that BCMA-CD19-VAC cCAR-T was expressed on roughly 28.18% of T-cells (FIG. 81). The other example CLL1-CD33-VAC cCAR-T construct bearing the large insert size does not hinder the expression, and that the single clone obtained by clone limiting dilution, expresses high levels of CAR on the T-cells was shown below.

CLL1-CD33 VAC cCAR (also called CLL1-CD33 IL-15/IL-15sushi cCAR or CLL1-CD33b VAC CAR) is a two-unit CAR composed of a complete CLL1-CAR fused to a complete CD33 CAR by a self-cleaving, enabling independent expression of both CAR receptors separately on the T cell surface. Each CAR contains a CD8 leader, a CD8-derived hinge (H) and transmembrane (TM) regions, and co-activation domains linked to the CD3ζ signaling domain. An enhancer, IL-15/IL-15sushi is also separated from the first CAR and second CAR by a second cleavage site at the C-terminus.

The construct of CLL1-CD33 VAC cCAR was transfected into the H29 cell line then, H29 producing-viruses were used to transduce RD114 cells. The highly expressed clones for

1. A method for treating an autoimmune disorder, the method comprising administering to a patient in need thereof a dual CAR, wherein the dual CAR binds to an antigen expressed on T-cells and an antigen on the surface of B-cells or plasma cells, wherein the antigen expressed on T cells is CD2, CD3, CD4, CD5, or CD7, and wherein the antigen on the surface of B cells or plasma cells is CD19, CD20, CD22, BCMA, CD38, CD138, CS1, or GPRC5D.

2. The method according to claim 1, wherein the autoimmune disorder is T-cell mediated.

3. The method according to claim 1, wherein the autoimmune disorder is T-cell and B-cell mediated.

4. The method according to claim 1, wherein the dual CAR is comprised of CD7CAR, and another CAR unit targeting CD19, CD20, CD22, BCMA, CD38, GPRC5D, or CS1, wherein administration to the patient in need thereof results in depletion of T-cells expressing CD7 surface antigen, or B-cells or plasma cell populations or a combination thereof.

5. The method according to claim 1, wherein the dual CAR binds to cells expressing CD7 or CD19.

6. The method according to claim 1, wherein the dual CAR binds to cells expressing CD7 or CD20.

7. The method according to claim 1, wherein the dual CAR binds to cells expressing CD7 or BCMA.

8. The method according to claim 1, wherein the method further comprises administering a steroid, or a B-cell inhibitory treatment, or a plasma cell inhibitory treatment or an immunosuppression treatment.

9. The method according to claim 1, wherein the autoimmune disorder is selected from Type I Diabetes, Multiple Sclerosis, Inflammatory Bowel Disease, Ulcerative Colitis, Crohn’s disease, Celiac’s disease, myasthenia gravis, Pemphigus vulgaris, Bullous pemphigoid Graves’ disease, Asthma, systemic lupus erythematosus, IgA nephropathy, IgG4 related disease, membranous nephropathy, Myasthenia gravis, Neuromyelitis optica, Pemphigus vulgaris, anti-PAD4-activating rheumatoid arthritis, sensitized/preformed antibodies in solid organ transplant, Psoriasis, Guillain-Barre Syndrome (Acute inflammatory demyelinating polyneuropathy -AIDP), Chronic inflammatory demyelinating polyneuropathy (CIDP), Evans syndrome, Immune thrombocytopenic purpura, rheumatoid arthritis, Sjogren’s syndrome, or ANCA-associated vasculitis (AAV).

10. The method according to claim 1, wherein the autoimmune disorder is selected from Type I diabetes, Multiple Sclerosis, Inflammatory Bowel Disease, Psoriasis, Ulcerative Colitis, or Crohn’s disease.

11. The method according to claim 1, wherein the autoimmune disorder is Type I. Diabetes.

12. The method according to claim 1, wherein the autoimmune disorder is Multiple Sclerosis.

13. The method according to claim 1, wherein the autoimmune disorder is Inflammatory Bowel Disease, Ulcerative Colitis, or Crohn’s disease.

14. The method according to claim 1, wherein the patient is at risk for developing a T-cell mediated autoimmune disorder or a combination of a T-cell and a B-cell mediated autoimmune disorder.

15. The method according claim 1, wherein the patient suffers from relapse or a refractory T-cell mediated autoimmune disorder or a combination of a T-cell and a B-cell mediated disorder.

16. The method according to claim 1, wherein the patient is at risk for auto-rejection of organ transplantation caused by autoreactive T-cells or autoreactive antibodies produced by B-cells or plasma cells.

17. A method for treating a cancer, the method comprising administering a CD7CAR combined with a CAR that requires CAR T-cell expansion to a patient in need thereof, wherein the CD7CAR is combined with a second CAR, wherein the second CAR targets at least one of GD2, GD3, ROR1, PSMA, PSCA (prostate stem cell antigen), MAGE A3, Glycolipid, glypican 3, F77, GD-2, WT1, CEA, HER-2/neu, MAGE-3, MAGE-4, MAGE-5, MAGE- 6, alpha-fetoprotein, CA 19-9, CA 72-4, NY-ESO, FAP, ErbB, c-Met, MART-1, MUC1, MUC2, MUC3, MUC4, MUC5, KIF20A, Survivin, AFP-1, gp100, MUC1, PAP-10, PAP-5, TRP2-1, SART-1, VEGFR1, VEGFR2, NEIL3, MPHOSPH1, DEPDC1, FOXM1, CDH3, TTK, TOMM34, URLC10, KOC1, UBE2T, TOPK, ECT2, MESOTHELIN, NKG2D, P1A, GM2, CD30, MMG49 epitope, EGFRvIII, CD33, CD123, CLL-1, immunoglobin kappa and lambda, CD38, CD52, CD47, CD200, CD70, CD19, CD20, CD22, CD38, BCMA, CS1, NKG2D receptor, April receptor, BAFF receptor, TACI, CD3, CD4, CD8, CD5, CD2, GPRC5D (G protein-coupled receptor, class C, group 5, member D), CD138, and viral or fungal antigens.

18. The method according to claim 1, wherein the dual CAR comprises an enhancer comprising IL-15/IL-15sushi, IL-15/IL-15sushi anchor, PD-1, PD-L1, CSF1R, CTAL-4, TIM-3, TGFR beta, IL-2, IL-7, IL-12, IL-15, CCL-19, CCL-21, IL-15RA, IL-21, functional fragments thereof, or combinations thereof.

19. The method according to claim 18, wherein the enhancer comprises IL-15/IL-15sushi.
